(19) United States
(12) Patent Application Publication
(10) Pub. No.: US 2026/0294827 A1
(43) Pub. Date: Oct. 1, 2026

(54) NANOPARTICLE COMPOSITION FOR OXYGEN DELIVERY

(71) Applicant: Swaza Inc., Mountain View, CA (US)

(72) Inventor: Jayakumar RAJADAS, Mountain View, CA (US)

(21) Appl. No.: 19/477,402

(22) PCT Filed: Apr. 26, 2024
(86) PCT No.: PCT/US2024/026675

§ 371 Date: Oct. 21, 2025

Related U.S. Application Data
(60) Provisional application No. 63/462,207, filed on Apr. 26, 2023.

(51) Int. Cl. A61K 9/51; A61P 11/00

(52) U.S. Cl. CPC A61K 9/5123; A61P 11/00

(57) ABSTRACT

The present disclosure in some aspects relates generally to nanoparticle compositions for oxygen delivery, method of use thereof, and method of preparation thereof. The present disclosure in some aspects relates generally to topical formulations capable of delivering oxygen to a tissue and methods of their use for treating diseases, such as hypoxemia.

### CROSS-REFERENCE TO RELATED APPLICATION

This application claims the priority benefit of U.S. Provisional Patent Application No. 63/462,207, filed on Apr. 26, 2023, the contents of which are hereby incorporated herein by reference in their entirety.

### FIELD

The present disclosure in some aspects relates generally to nanoparticle compositions for oxygen delivery, method of use thereof, and method of preparation thereof. The present disclosure in some aspects relates generally to formulations capable of delivering oxygen to a tissue and methods of their use for treating diseases, such as hypoxemia.

### BACKGROUND

Acute Respiratory Distress Syndrome (ARDS) is a life-threatening condition characterized by the failure of O₂ and CO₂ movement (gas exchange) across the alveolar-capillary membranes. The injuries resulting from ARDS in the lung cause fluid accumulations in the alveolar units of the lung thus preventing oxygen and carbon dioxide transport. The fluid accumulation impedes the rate of oxygen and carbon dioxide exchange at the alveolar epithelial cells and the capillaries resulting in lack of oxygenated blood in the system.

ARDS carries a high degree of morbidity and frequently leads to long-lasting health complications. The respiratory complications of traumatic injury, direct exposure to chemical and/or biological warfare agents, or pandemic respiratory viral illnesses pose a serious threat to operational success, particularly in resource-limited settings. Clinical management of ARDS is supportive and involves the use of adjunctive measures to correct critical hypoxemia such as mechanical ventilation for respiratory failure, systemic corticosteroids to reduce inflammation and, if available, extracorporeal life support (ECLS) to deliver O₂ and remove CO₂ directly from the blood. Despite the availability of these adjuncts, mechanical ventilation may result in cellular-level trauma as the alveoli are stretched and deformed under positive pressure, thus contributing to additional lung damage; corticosteroids reduce inflammation but the underlying inflammatory processes leading to ARDS remain; and ECLS is not available in most hospitals. Consequently, there is a strong need for new or refined treatment options for ARDS-associated refractory hypoxemia and hypercapnia, particularly at the point when the lungs are no longer able to effectively facilitate normal O₂ and CO₂ transport.

To develop more efficient treatment options for hypoxemia, oxygen delivery compositions have been developed, especially formulations comprising perfluorocarbons. Perfluorocarbons are class of molecules consisting mainly of fluorine and carbon. They are characterized by high gas solubility, fast release, high volume-quality, average volatility, good histocompatibility and the absence of absorption and metabolism in vivo. Because of their capability of dissolving large amounts of physiologically important gases (mainly oxygen and carbon dioxide), many artificial oxygen carriers based on perfluorocarbon emulsions have been developed over the past century. However, there remains a need to develop more efficient, stable, and safer perfluorocarbon-based compositions for potent O₂ delivery and hypoxemia treatment. The present application addresses this and other need.

### BRIEF SUMMARY OF THE INVENTION

In some aspects, provided herein is a nanoparticle composition for oxygen delivery, comprising nanoparticles, wherein the nanoparticles comprise a perfluorocarbon component and a lipid component, wherein the perfluorocarbon component comprises perfluorodecalin (PFD) and perflurotripropylamine (PFTPA), and optionally wherein the nanoparticle composition comprises no more than about 2% (w/w) polymeric surfactant of the total nanoparticle composition.

In some embodiments, the perfluorocarbon component comprises (e.g., is) a mixture of perfluorodecalin (PFD) and perflurotripropylamine (PFTPA), optionally at a weight ratio of about 2:1 to about 3:1. In some embodiments, the lipid component comprises (e.g., is) a mixture of DSPC and DPPC, optionally at a weight ratio of about 1:1 to about 4:1. In some embodiments, the lipid component comprises no more than about 2% (w/w) hydrogenated soya phosphatidyl choline (HSPC) of the total nanoparticle composition. In some embodiments, the nanoparticle composition further comprises a buffer component. In some embodiments, the nanoparticle composition comprises a buffer comprising the buffer component and water. In some embodiments, the buffer component comprises sodium citrate hydrate and citric acid at a weight ratio of about 5:1 to about 20:1, optionally 12:1. In some embodiments, the weight ratio between the perfluorocarbon component and the buffer component is about 3.5:1 to about 4.2:1, such as about 3.8:1. In some embodiments, the buffer comprises a citrate at about 20 mM to about 100 mM, optionally at a weight percentage of about 40% to about 60% (w/w) of the total nanoparticle composition. In some embodiments, the nanoparticle composition comprises no more than about 2% (w/w) phosphate buffer of the total nanoparticle composition. In some embodiments, the perfluorocarbon component is about 40% to about 60% (w/w) of the total nanoparticle composition. In some embodiments, the lipid component is about 1.5% to about 3.5% (w/w) of the total nanoparticle composition. In some embodiments, the nanoparticle composition has a pH of about 3.5 to about 8.0. In some embodiments, the nanoparticle composition has a viscosity of no more than about 100 cp. In some embodiments, the average particle size of the nanoparticles is no more than about 3 μm. In some embodiments, the nanoparticles, when diluted with 5× (v/v) citrate buffer, have an average particle size of no more than about 500 nm. In some embodiments, the nanoparticles have a multi lamellar structure comprising at least two lamellae layers, wherein each lamellae layer comprises the perfluorocarbon sandwiched by two layers of the lipid. In some embodiments, the nanoparticle composition is enriched with at least about 5 ppm dissolved oxygen. In some embodiments, the nanoparticle composition is stable for at least 4 days under room temperature without phase separation.

In some embodiments, the nanoparticle composition comprises no more than about 2% (w/w) centrimonium bromide (CTAB) of the total nanoparticle composition. In some embodiments, the nanoparticle composition comprises no more than about 2% (w/w) Tween 80 of the total nanoparticle composition. In some embodiments, the nanoparticle composition comprises no more than about 2% (w/w) Soya Lecithin of the total nanoparticle composition. In some embodiments, the nanoparticle composition comprises no more than about 2% (w/w) poloxamer of the total nanoparticle composition. In some embodiments, the nanoparticle composition comprises no more than about 2% (w/w) surfactant or lipid other than DSPC and DPPC. In some embodiments, the nanoparticle composition is suitable for inhalation, topical administration, or enema administration.

In some embodiments, the nanoparticle composition comprises (i) PFD at a weight percentage of about 35.5% of the total nanoparticle composition; (ii) PFTPA at a weight percentage of about 14.5% of the total nanoparticle composition; (iii) DPPC at a weight percentage of about 0.6% of the total nanoparticle composition; (iv) DSPC at a weight percentage of about 1.7% of the total nanoparticle composition; and (v) pH 6.0 Citrate buffer (50 mM) at a weight percentage of about 47.7% of the total nanoparticle composition.

In some embodiments, the composition comprises (i) PFD at a weight percentage of about 35.5% of the total nanoparticle composition; (ii) PFTPA at a weight percentage of about 14.5% of the total nanoparticle composition; (iii) DPPC at a weight percentage of about 0.6% of the total nanoparticle composition; (iv) DSPC at a weight percentage of about 1.7% of the total nanoparticle composition; (v) citric acid at a weight percentage of about 0.1% of the total nanoparticle composition (vi) sodium citrate dihydrate at a weight percentage of about 1.2% of the total nanoparticle composition; and (viii) water at a weight percentage of about 46.4% of the total nanoparticle composition.

In some aspects, provided herein is a wound healing composition comprising the nanoparticle composition described herein, silver dioxide, and a collagen. In some aspects, provided herein is an enema gel comprising the nanoparticle composition described herein, a poloxamer, and a PBS solution. In some aspects, provided herein is a cream composition comprising the nanoparticle composition described herein, and a pharmaceutically acceptable excipient.

In some aspects, provided herein is a method of delivering a tissue experiencing a reduction in normal oxygen levels (such as determined compared to a healthy state as measured by techniques known in the art) in a subject in need thereof, comprising administering the nanoparticle composition described herein, the wound healing composition described herein, the enema gel described herein, or the cream composition described herein, to the subject. In some embodiments, the tissue is at the skin, in the colon or GI, or in the lung. In some embodiments, provided herein is a method of treating a condition in a subject in need thereof, comprising topically administering the nanoparticle composition described herein, the wound healing composition described herein, the enema gel described herein, or the cream composition described herein, to the subject. In some embodiments, the condition is a wound, an open injury, a burn, a colon or GI condition, or a pulmonary condition. In some embodiments, the pulmonary condition is COVID, chronic obstructive pulmonary disease (COPD), asthma, or lung injury. In some embodiments, the condition is ARDS. In some embodiments, the ARDS is caused by bacterial infection.

### BRIEF DESCRIPTION OF THE DRAWINGS

The drawings illustrate certain embodiments of the features and advantages of this disclosure. These embodiments are not intended to limit the scope of the appended claims in any manner.

FIG. 1 shows an illustrative scheme of the gas exchange process achieved by nanoparticles in a nanoparticle composition.

FIG. 2 shows microscopic images of the nanoparticles in a nanoparticle composition.

FIG. 3A shows microscopic images of the nanoparticles in nanoparticle composition NF-62. FIG. 3B shows microscopic images of the nanoparticles in nanoparticle composition NF-66.

FIG. 4 shows processes of low-energy (high pressure homogenization/ultrasonication) and high-energy method (microfluidization) processes for preparing nanoparticle compositions.

FIG. 5A shows microscopic images of lung tissues in mice with ARDS and treated with nanoparticle composition NF-83. FIG. 5B shows microscopic images of untreated lung tissues in mice with ARDS.

FIG. 6 shows microscopic images of lung tissues in mice with bacterial infection-induced ARDS. The upper panel shows lung tissues in untreated and normal mice. The bottom left panel shows the lung tissues in untreated and diseased mice. The bottom right panel shows the image of lung tissues in diseased mice treated with nanoparticle composition NF-83.

FIG. 7A illustrates the trajectory of nanoparticles in nanoparticle composition NF-83 as they passed through microchannels. FIGS. 7B and 7C illustrate the trajectory of individual nanoparticles in nanoparticle composition NF-83 over 0-10 seconds and 0-30 seconds, respectively.

FIG. 8 shows sample images of the nanoparticles.

FIG. 9A shows the level of CFTR1 expression in the lung tissue of mice treated with LPS. The saline group was used as a control and the formulation group was administered with nanoparticle composition NF-83. FIG. 9B shows the level of TRPV1 expression in lung tissues in mice treated with LPS. The saline group was used as a control, and the formulation group was administered with nanoparticle composition NF-83. FIGS. 9C and 9D show fluorescence (representing COX-PTGS2 expression level) in lung tissues of mice treated with LPS. The control group referred to untreated and normal mice. The untreated group referred to untreated and diseased mice. The treated group referred to diseased mice treated with nanoparticle composition NF-83.

FIG. 10 shows the conformational change of collagen-induced by an exemplary nanoparticle composition.

FIG. 11 shows images of lung tissues distributed with nanoparticle composition stained with Texas red.

FIG. 12 shows the quench of Fluorescein (FITC) by exemplary nanoparticle composition.

### DETAILED DESCRIPTION

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

### I. Definition

As used in the present specification, the following words and phrases are generally intended to have the meanings as set forth below, except to the extent that the context in which they are used indicates otherwise.

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, where applicable, the terms “about” and “approximately,” when used in this context, contemplate a dose, amount, or weight percent within 15% of the specified dose, amount, or weight percent.

The terms “patient,” “subject,” “individual,” and the like are used interchangeably herein, and refer to any animal, in some embodiments a mammal, and in some embodiments a human, including a human in need of therapy for, or susceptible to, a condition or its sequelae. The individual may include, for example, dogs, cats, pigs, cows, sheep, goats, horses, rats, rabbits, hamsters, guinea pigs, monkeys, mice, and humans. In some embodiments, the individual is a human. In some embodiments, the individual is an animal.

The term “treatment” typically refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with disease or disorder are mitigated or eliminated, including, but not limited to, decreasing the frequency and/or severity of a sign and/or symptoms resulting from the disease, increasing the quality of life of those suffering from the disease, decreasing the dose of other medications required to treat the disease, and/or prolonging survival of individuals. Treatment may be prophylactic (to prevent or delay the onset of the disease, or to prevent the manifestation of clinical or subclinical symptoms thereof) or therapeutic suppression or alleviation of symptoms after the manifestation of the disease.

The term “therapeutically effective amount” or “effective amount” refers to that amount of a composition or formulation described herein that is sufficient to affect treatment, as defined herein, when administered to a patient in need of such treatment. A therapeutically effective amount of a composition or formulation may be an amount sufficient to treat a disease associated with lack of oxygen. The therapeutically effective amount will vary depending upon, for example, the subject and disease condition being treated, the weight and age of the subject, the severity of the disease condition, the particular compound, the dosing regimen to be followed, timing of administration, the manner of administration, all of which can readily be determined by one of ordinary skill in the art. The therapeutically effective amount may be ascertained experimentally, for example by assaying blood concentration of the chemical entity, or theoretically, by calculating bioavailability.

In some embodiments, as used herein, “polymeric” refers to the structural features similar to those of a polymer. “Polymer” typically refers to a very large molecule such as a macromolecule, which comprises many repeating subunits. In some embodiments, a polymer refers to a molecule with one or more repeating unit and having a molecular weight of at least about 2500 Dalton, at least about 3000 Dalton, at least about 4000 Dalton, or at least about 5000 Dalton. In some embodiments, a polymer does not encompass lipid. In some embodiments, a polymeric surfactant does not encompass lipid surfactant

In some embodiments, the disclosure also includes any or all of the stereochemical forms of compound provided herein (e.g., perfluorocarbon and lipid), including any enantiomeric or diastereomeric forms, and any tautomers or other forms of the compounds described. In some embodiments, unless stereochemistry is explicitly indicated in a chemical structure or name, the structure or name is intended to embrace all possible stereoisomers of a compound depicted. Compositions comprising a mixture of compounds described herein in any ratio are also embraced by the disclosure, including mixtures of two or more stereochemical forms of a compound in any ratio, such that racemic, non-racemic, enantioenriched and scalemic mixtures of a compound are embraced. Where one or more tertiary amine moiety is present in the compound, the N-oxides are also provided and described.

In some embodiments, the disclosure also includes further isotopically-labeled and/or isotopically-enriched forms of compounds described herein (e.g., perfluorocarbon and lipid). The compounds herein may contain unnatural proportions of atomic isotopes in addition to deuterium at one or more of the atoms that constitute such compounds. Exemplary additional isotopes that can be incorporated into compounds described herein include isotopes of hydrogen, carbon, nitrogen, oxygen, sulfur, chlorine, such as ²H, ³H, ¹¹C, ¹³C, ¹⁴C ¹³N, ¹⁵O, ¹⁷O, ³⁵S, ¹⁸F, ³⁶Cl. As used herein, each instance of enrichment, substitution, or replacement of an atom with corresponding isotope of that atom encompasses isotopic enrichment levels of one of about: 50%, 60%, 70%, 80%, 90%, 95%, 96%, 97%, 98%, 99%, 99.5%, 99.6%, 99.7%, 99.8%, 99.9%, or 100%, or a range between any two of the preceding percentages.

As used herein, the term “substantially free of” means that the composition contains no more than 15%, no more than 10%, no more than 5%, no more than 4%, no more than 3%, no more than 2%, or no more than 1% by weight of the indicated substance or substances.

It is understood that embodiments described herein as “comprising” include “consisting of” and “consisting essentially of” embodiments.

### II. Nanoparticle Composition

In one aspect, provided herein is a nanoparticle (e.g., nano-emulsion) composition for oxygen delivery, comprising nanoparticles, wherein the nanoparticles comprise a perfluorocarbon component and a lipid component. In some embodiments, the nanoparticle (e.g., nano-emulsion) composition comprises no more than about 2% (w/w) polymeric surfactant. For example, in some embodiments, the nanoparticle composition is substantially free (e.g., completely free) of polymeric surfactants. In some embodiments, the nanoparticle (e.g., nano-emulsion) composition comprises nanoparticles, wherein the nanoparticles comprise perfluorodecalin (PFD), perflurotripropylamine (PFTPA), DSPC, DPPC, and the nanoparticle composition comprises a buffer, wherein the nanoparticle (e.g., nano-emulsion) composition is substantially free of surfactant other than lipid, and the nanoparticle (e.g., nano-emulsion) composition is stable (e.g., does not have phase separation) at room temperature for at least four days. In some embodiments, the nanoparticle (e.g., nano-emulsion) composition comprises nanoparticles, wherein the nanoparticles comprise perfluorodecalin (PFD), perflurotripropylamine (PFTPA), DSPC, DPPC, and the nanoparticle composition comprises a buffer component, optionally wherein the buffer component comprises sodium citrate hydrate and citric acid. In some embodiments, the buffer component comprises sodium citrate hydrate and citric acid at a weight ratio of about 5:1 to about 20:1, such as about any of 8:1, 9:1, 10:1, 11:1, 12:1, 13:1, 14:1, 15:1, or any value between the preceding values. In some embodiments, the PFD and PFTPA are at a weight ratio of about 2:1 to about 3:1. In some embodiments, DSPC and DPPC are at a weight ratio of about 1:1 to about 4:1. In some embodiments, the perfluorocarbon component is about 40% to about 60% (w/w) of the total nanoparticle (e.g., nano-emulsion) composition and the lipid component is about 1.5% to about 3.5% (w/w) of the total nanoparticle (e.g., nano-emulsion) composition. In some embodiments, the buffer comprises a citrate at about 50 mM, optionally at a weight percentage of about 40% to about 60% (w/w) of the total nanoparticle (e.g., nano-emulsion) composition. In some embodiments, perfluorocarbon component and buffer component are at a weight ratio of about 2.5:1 to about 5, for example, any of about 3:1 to about 4.5:1, about 3.5:1 to about 4.5:1, or about 3.8:1 in the nanoparticle composition. In some embodiments, the nanoparticle (e.g., nano-emulsion) composition has a pH of about 5.0 to about 8.0, such as about 6.0 to about 7.0. In some embodiments, the nanoparticle (e.g., nano-emulsion) composition has a viscosity of no more than about 100 cp. In some embodiments, the nanoparticles, when diluted with 5× (v/v) citrate buffer, have an average particle size of no more than about 500 nm, such as about 200 nm to about 500 nm. In some embodiments, the nanoparticle (e.g., nano-emulsion) composition is enriched with at least about 5 ppm dissolved oxygen. In some embodiments, the nanoparticle (e.g., nano-emulsion) composition is stable for at least 4 days, such as at least 7 days or 10 days under room temperature without phase separation. In some embodiments, the nanoparticle (e.g., nano-emulsion) composition is substantially free of hydrogenated soya phosphatidyl choline (HSPC), centrimonium bromide (CTAB), Tween 80, Soya Lecithin, or other surfactant or lipid other than DSPC and DPPC. In some embodiments, the nanoparticle (e.g., nano-emulsion) compositions are suitable for topical administration, such as inhalation.

In some embodiments, the nanoparticle (e.g., nano-emulsion) compositions provided herein are efficient in oxygen delivery, which compared to the known perfluorocarbon emulsions have at least several key advantages. First of all, the known perfluorocarbon emulsion tends to have low stability, especially under room temperature. For example, despite a low concentration of perfluorocarbon, artificial blood fluosol can only be stored under frozen state, which significantly hinders its use, storage, and transport. In contrast, the nanoparticle (e.g., nano-emulsion) compositions provided herein are stable (e.g., does not have phase separation) under room temperature for at least a day. Furthermore, in order to improve the stability, current perfluorocarbon emulsions usually have polymeric surfactants, such as poloxamer, which may induce complement activation. Being substantially free of polymeric surfactants, the nanoparticle compositions provided herein become safer and less irritative. In addition, the known perfluorocarbon emulsions are typically administered intravenously, which increases the operational difficulty and cost. Therefore, the nanoparticle (e.g., nano-emulsion) compositions described herein are crucial in that they can deliver O₂ efficiently while being stable, non-toxic and can be administered in a non-invasive and convenient way, such as topical administration.

### Perfluorocarbon

In some embodiments, the perfluorocarbon component comprises a perfluoroalkyl. In some embodiments, the perfluoroalkyl is an alkyl wherein all hydrogens are independently substituted with F or Br. In some embodiments, the alkyl is a C₄-C₂₀. In some embodiments, the alkyl is cyclic, acyclic, branched, or unbranched. In some embodiments, the perfluorocarbon component comprises a tri-perfluoroalkylamine, wherein the perfluorinated alkyl group is cyclic, acyclic, branched, or unbranched. In some embodiments, the perfluoronated alkyl group is C₁-C₁₅. In some embodiments, the tri-perfluoroalkylamine can be protonated. In some embodiments, the perfluorocarbon component comprises perfluorobutane, perfluropentane, perfluoro tert-buylcyclohexane, perfluoro-2-methylhexane, perfluorodecalin (PFD), perflurotripropylamine (PFTPA), perflurotributylamine, perflurotripentylamine, perfluorotrihexylamine, perfluoro-N-(4-methylcyclohexyl) piperidine, perfluoro-N-methyl-piperidine, perfluoro-dicyclohexyl-methyl amine, perfluorooctylbromide, perfluorodecylbromide, or any combination thereof. In some embodiments, the perfluorocarbon component comprises perfluorodecalin (PFD), perflurotripropylamine (PFTPA), perflurotributylamine, perflurotripentylamine, perfluorobutane, or any combination thereof. In some embodiments, the perfluorocarbon component comprises PFD. In some embodiments, the perfluorocarbon component comprises PFTPA. In some embodiments, the perfluorocarbon component comprises PFD and PFTPA. In some embodiments, the perfluorocarbon component consists essentially of PFD and PFTPA. In some embodiments, the perfluorocarbon component comprises no more than about 2% (w/w), for example, no more than about any of 1.5% (w/w), 1.0% (w/w), 0.5% (w/w), 0.1% (w/w), 0.01% (w/w), or 0.001% (w/w) perfluorocarbon other than PFD and PFTPA. In some embodiments, the perfluorocarbon component consists of PFD and PFTPA. In some embodiments, the PFD and PFTPA in the nanoparticle (e.g., nano-emulsion) composition are at a weight ratio of about 2:1 to about 3:1, for example, any of about 2:1 to about 2.8:1, about 2:1 to about 2.6:1, about 2.2:1 to about 2.6:1, or about 2.4:1.

In some embodiments, the weight percentage of perfluorocarbon component in the total nanoparticle (e.g., nano-emulsion) composition is at least about 20% (w/w), for example, at least about any of 24% (w/w), 26% (w/w), 28% (w/w), 30% (w/w), 32% (w/w), 34% (w/w), 36% (w/w), 40% (w/w), 42% (w/w), 44% (w/w), 46% (w/w), 48% (w/w), 50% (w/w), or 55% (w/w). In some embodiments, weight percentage of perfluorocarbon component in the total nanoparticle (e.g., nano-emulsion) composition is no more than about 90% (w/w), for example, no more than about any of 88% (w/w), 86% (w/w), 84% (w/w), 82% (w/w), 80% (w/w), 78% (w/w), 76% (w/w), 72% (w/w), 70% (w/w), 68% (w/w), 66% (w/w), 64% (w/w), 62% (w/w), or 60% (w/w). In some embodiments, weight percentage of perfluorocarbon component in the total nanoparticle (e.g., nano-emulsion) composition is about 20% to about 90% (w/w), for example, any of about 25% to about 85% (w/w), about 30% to about 70% (w/w), about 40% to about 50% (w/w), or about 50% (w/w).

### Lipid

In some embodiments, the lipid component comprises a phospholipid, for example, more than one types of phospholipids. In some embodiments, the phospholipid can be naturally occurring or synthetically derived. In some embodiments, the lipid component comprises comprises phoshatidic acid, phosphytidylethanolamine, phosphaidylcholine, phosphatidylserine, phosphatidyl inositol, distearoylphosphatidylcholine (DSPC), dipalmitoylphosphatidylcholine (DPPC), dioleoylphosphatidylcholine, dimyristoylphosphatidylcholine (DMPC), palmitoyloleoylphosphatidylcholine (POPC), phosphatidylethanolamine, sphingomyelin, phosphatidylserine and phosphatidylinositol, fatty mixtures containing glycerolphospholipids such as soya lecithin, soya phosphatidyl choline (HSPC), 1-oleoyl-2-palmitoyl-sn-glycero-3-phosphocholine (OPPC), 1,2-dioleoyl-sn-glycero-3-phosphocholine (DOPC), 2-dierucoyl-sn-glycero-3-phosphocholine (DEPC), or any combination thereof.

In some embodiments, the lipid component comprises DSPC and DPPC. In some embodiments, the lipid component consists essentially of DSPC and DPPC. In some embodiments, the lipid component comprises no more than about 2% (w/w), for example, no more than about any of 1.5% (w/w), 1.0% (w/w), 0.5% (w/w), 0.1% (w/w), 0.01% (w/w), or 0.001% (w/w) lipid other than DSPC or DPPC. In some embodiments, the lipid component comprises no more than about 2% (w/w), for example, no more than about any of 1.5% (w/w), 1.0% (w/w), 0.5% (w/w), 0.1% (w/w), 0.01% (w/w), or 0.001% (w/w) soya lecithin. In some embodiments, the total nanoparticle (e.g., nano-emulsion) composition comprises no more than about 2% (w/w), for example, no more than about any of 1.5% (w/w), 1.0% (w/w), 0.5% (w/w), 0.1% (w/w), 0.01% (w/w), or 0.001% (w/w) soya lecithin of the total nanoparticle (e.g., nano-emulsion) composition. In some embodiments, the lipid component comprises no more than about 2% (w/w), for example, no more than about any of 1.5% (w/w), 1.0% (w/w), 0.5% (w/w), 0.1% (w/w), 0.01% (w/w), or 0.001% (w/w) HSPC. In some embodiments, the total nanoparticle (e.g., nano-emulsion) composition comprises no more than about 2% (w/w), for example, no more than about any of 1.5% (w/w), 1.0% (w/w), 0.5% (w/w), 0.1% (w/w), 0.01% (w/w), or 0.001% (w/w) HSPC of the total nanoparticle (e.g., nano-emulsion) composition.

In some embodiments, the lipid component comprises (e.g., is) a mixture of DSPC and DPPC at a weight ratio of about 1:1 to about 5:1, for example, any of about 1:1 to about 4.5:1, about 1:1 to about 4:1, about 1.5:1 to about 4:1, about 2:1 to about 4:1, about 2:1 to about 3.5:1, or about 2.8:1. In some embodiments, the lipid component comprises (e.g., is) a mixture of DSPC and DPPC at a weight ratio of about 2.8:1.

In some embodiments, the weight percentage of the lipid component in the total nanoparticle (e.g., nano-emulsion) composition is at least about 0.5% (w/w), for example, at least about any of 0.8% (w/w), 1.0% (w/w), 1.2% (w/w), 1.4% (w/w), 1.6% (w/w), 2% (w/w), 2.2% (w/w), 2.4% (w/w), 2.6% (w/w), 2.8% (w/w), or 3% (w/w). In some embodiments, the weight percentage of the lipid component in the total nanoparticle (e.g., nano-emulsion) composition is no more than 4.5% (w/w), for example, at least about any of 4.2% (w/w), 4% (w/w), 3.8% (w/w), 3.5% (w/w), 3.2% (w/w), 3% (w/w), 2.8% (w/w), 2.6% (w/w), or 2.5% (w/w). In some embodiments, the weight percentage of the lipid component in the total nanoparticle (e.g., nano-emulsion) composition is about 1.5% to about 3.5% (w/w), for example, any of 2.0% to about 3.5% (w/w), 2.0% to about 3.0% (w/w), 2.0% to about 2.5% (w/w), or about 2.3%.

In some embodiments, perfluorocarbon component and lipid component are at a weight ratio of at least about 5:1, for example, at least about any of 6:1, 7:1, 8:1, 9:1, 10:1, 12:1, 14:1, 16:1, 18:1, 20:1, or 22:1 in the nanoparticle composition. In some embodiments, perfluorocarbon component and lipid component are at a weight ratio of no more than about 50:1, for example, no more than about any of 45:1, 40:1, 35:1, 30:1, 25:1, or 23:1. In some embodiments, perfluorocarbon component and lipid component are at a weight ratio of about 5:1 to about 40:1, for example, any of about 10:1 to about 30:1, about 15:1 to about 23:1, or about 23:1 in the nanoparticle composition.

### Buffer and Buffer Component

In some embodiments, the nanoparticle composition further comprises a buffer component. In some embodiments, the nanoparticle composition further comprises a buffer comprising a buffer component and water. In some embodiments, the buffer can maintain a stable pH and/or control tonicity. In some embodiments, the buffer comprises an acetate, formate, citrate, glutamate or a phosphate buffer, or any combination thereof. In some embodiments, the buffer comprises a citrate buffer. In some embodiments, the buffer comprises a citrate buffer at about 20 mM to about 100 mM, such as about any of 30 mM to 70 mM, or 50 mM. In some embodiments, the buffer comprises a citrate buffer at 50 mM. In some embodiments, the citrate buffer is at pH 6.0. In some embodiments, the buffer component comprises sodium citrate hydrate and citric acid at a weight ratio of about 5:1 to about 20:1, such as about any of 8:1, 9:1, 10:1, 11:1, 12:1, 13:1, 14:1, 15:1, or any value between the preceding values. In some embodiments, the buffer component comprises sodium citrate hydrate and citric acid at a weight ratio such that the buffer has a pH of about 4 to about 8, such as about 6.

In some embodiments, the buffer comprises a phosphate buffer at pH 6.8. In some embodiments, the buffer does not comprise a phosphate buffer. In some embodiments, the buffer comprises no more than about 2% (w/w), for example, no more than about any of 1.5% (w/w), 1.0% (w/w), 0.5% (w/w), 0.1% (w/w), 0.01% (w/w), or 0.001% (w/w) phosphate buffer. In some embodiments, the total nanoparticle (e.g., nano-emulsion) composition comprises no more than about 2% (w/w), for example, no more than about any of 1.5% (w/w), 1.0% (w/w), 0.5% (w/w), 0.1% (w/w), 0.01% (w/w), or 0.001% (w/w) phosphate buffer.

In some embodiments, the buffer is at least about 20% (w/w), for example, at least about any of 22% (w/w), 25% (w/w), 27% (w/w), 30% (w/w), 32% (w/w), 35% (w/w), 37% (w/w), 40% (w/w), 42% (w/w), 44% (w/w), or 46% (w/w) of the total nanoparticle (e.g., nano-emulsion) composition. In some embodiments, the buffer is no more than 70% (w/w), for example, no more than any of 68% (w/w), 65% (w/w), 63% (w/w), 60% (w/w), 57% (w/w), 55% (w/w), 52% (w/w), 50% (w/w), or 48% (w/w) of the total nanoparticle (e.g., nano-emulsion) composition. In some embodiments, the buffer is about 20% to about 70% (w/w), for example, any of about 30% to about 60% (w/w), about 30% to about 50% (w/w), about 40% to about 60% (w/w), or about 48% (w/w) of the total nanoparticle (e.g., nano-emulsion) composition.

In some embodiments, the buffer component comprises sodium citrate dihydrate, and the weight percentage of the sodium citrate dihydrate in the total nanoparticle (e.g., nano-emulsion) composition is at least about 5% (w/w), for example, at least about any of 6% (w/w), 7% (w/w), 8% (w/w), 9% (w/w), 10% (w/w), 11% (w/w), or 12% (w/w). In some embodiments, the buffer component comprises sodium citrate dihydrate, and the weight percentage of the sodium citrate dihydrate in the total nanoparticle (e.g., nano-emulsion) composition is no more than 10% (w/w), for example, no more than any of 11% (w/w), 12% (w/w), 13% (w/w), 14% (w/w), 15% (w/w), 16% (w/w), or 20% (w/w). In some embodiments, the buffer component comprises sodium citrate dihydrate, and the weight percentage of the sodium citrate dihydrate in the total nanoparticle (e.g., nano-emulsion) composition is about 5% to about 20% (w/w), for example, any of about 7% to about 15% (w/w), or about 12% (w/w).

In some embodiments, the buffer component comprises citric acid, and the weight percentage of the citric acid in the total nanoparticle (e.g., nano-emulsion) composition is at least about 0.001% (w/w), for example, at least about any of 0.003% (w/w), 0.005% (w/w), 0.007% (w/w), 0.009% (w/w), 0.1% (w/w), 0.12% (w/w), or 0.15% (w/w). In some embodiments, the buffer component comprises citric acid, and the weight percentage of the citric acid the total nanoparticle (e.g., nano-emulsion) composition is no more than 0.2% (w/w), for example, no more than any of 0.18% (w/w), 0.16% (w/w), 0.14% (w/w), 0.12% (w/w), or 0.1% (w/w). In some embodiments, the buffer component comprises citric acid, and the weight percentage of the citric acid in the total nanoparticle (e.g., nano-emulsion) composition is about 0.01% to about 0.2% (w/w), for example, any of about 0.05% to about 0.15% (w/w), or about 0.1% (w/w).

In some embodiments, the perfluorocarbon component and buffer are at a weight ratio of at least about 0.5:1, for example, at least about any of 0.75:1, 0.9:1, 1:1, 9:1, or 1.1:1. In some embodiments, the perfluorocarbon component and buffer are at a weight ratio of no more than about 2:1, for example, no more than about any of 1.8:1, 1.7:1, 1.5:1, 1.4:1, 1.3:1, 1.1:1, or 1:1. In some embodiments, perfluorocarbon component and lipid component are at a weight ratio of about 0.8:1 to about 2:1, for example, any of about 0.8:1 to about 1.5:1, about 0.8:1 to about 1.3:1, or about 1.1:1.

In some embodiments, the buffer comprises a buffer component and water. In some embodiments, the buffer component comprises sodium citrate hydrate and citric acid at a weight ratio of about 5:1 to about 20:1, such as about any of 8:1, 9:1, 10:1, 11:1, 12:1, 13:1, 14:1, 15:1, or any value between the preceding values. In some embodiments, the buffer component comprises sodium citrate hydrate and citric acid at a weight ratio such that the buffer has a pH of about 4 to about 8, such as about 6. In some embodiments, the perfluorocarbon component and buffer component are at a weight ratio of at least about 1:1, for example, at least about any of 1.5:1, 2:1, 2.5:1, 3:1, or 3.5:1. In some embodiments, the perfluorocarbon component and buffer component are at a weight ratio of no more than about 6:1, for example, no more than about any of 5.5:1, 5:1, 4.5:1, 4:1, or 3.5:1. In some embodiments, perfluorocarbon component and buffer component are at a weight ratio of about 2.5:1 to about 5, for example, any of about 3:1 to about 4.5:1, about 3.5:1 to about 4.5:1, or about 3.8:1.

In some embodiments, the nanoparticle (e.g., nano-emulsion) composition described herein may be diluted by a buffer (e.g., a citrate buffer), wherein the diluted nanoparticle (e.g., nano-emulsion) composition is also encompassed by the present invention. In some embodiments, the nanoparticle composition can be diluted by at least 1× (v/v) (e.g., about any of 1× (v/v), 2× (v/v), 3× (v/v), 4× (v/v), 5× (v/v), 6× (v/v), or 7× (v/v)) citrate buffer. In some embodiments, provided herein is composition comprising the nanoparticle (e.g., nano-emulsion) composition described herein and additional citrate buffer, wherein the volumetric ratio of the additional citrate buffer to the nanoparticle (e.g., nano-emulsion) composition is at least 0.5:1 (e.g., about any of 1:1, 2:1, 3:1, 4:1, 5:1, 6:1, 7:1, or 8:1).

### Characteristics

In some embodiments, the nanoparticle (e.g., nano-emulsion) composition has a pH at least about 4, for example, at least about any of 4, 4.5, 5, or 5.5. In some embodiments, the nanoparticle (e.g., nano-emulsion) composition has a pH no more than about 9, for example, no more than about any of 8.5, 8, 7.5, 7, 6.5, or 6. In some embodiments, the nanoparticle (e.g., nano-emulsion) composition has a pH of about 3.5 to about 8, for example, any of about 4 to 8, about 5 to about 8, about 5 to about 7.5, or about 6 to about 7.5.

In some embodiments, the nanoparticle (e.g., nano-emulsion) composition has a viscosity of no more than about 500 cp, for example, no more than about any of 400 cp, 300 cp, 200 cp, 100 cp, 80 cp, 60 cp, 40 cp, or 20 cp, as determined by viscometer.

In some embodiments, the average particle size of the nanoparticles is no more than about 5 μm (e.g., no more than about any of 4 μm, 3 μm, 2 μm, 1 μm, or 500 nm). In some embodiments, the average particle size of the nanoparticles in the nanoparticle (e.g., nano-emulsion) composition can be further reduced by diluting the nanoparticle (e.g., nano-emulsion) composition with the buffer. In some embodiments, the nanoparticle (e.g., nano-emulsion) composition can be further diluted by at least about 0.5× (e.g., at least about 1×, 2×, 3×, 4×, 5×, or 6×) citrate buffer. In some embodiments, the nanoparticle (e.g., nano-emulsion) composition can be diluted by 5× citrate buffer and the average particle size of the nanoparticles in the diluted nanoparticle (e.g., nano-emulsion) composition is no more than about 1 μm (e.g., about any of 50 nm to about 900 nm, 50 nm to about 700 nm, 50 nm to about 600 nm, 50 nm to about 400 nm, or 200 nm to about 400 nm). In some embodiments, the polydispersity index (PDI) of the nanoparticles in the 5× citrate diluted nanoparticle (e.g., nano-emulsion) composition is about 0.5 to about 1 (e.g., about any of 0.6 to 1, 0.7 to 1, 0.8 to 1, 0.9 to 1, or 1). In some embodiments, the zeta potential of the nanoparticles in the 5× citrate diluted nanoparticle (e.g., nano-emulsion) composition is about 0.02 mV to about 0.8 mV (e.g., about any of 0.02 mV to 0.8 mV, 0.02 mV to 0.7 mV, 0.02 mV to 0.2 mV, or 0.5 mV to 0.7 mV). In some embodiments, the average particle size of the nanoparticles in the nanoparticle (e.g., nano-emulsion) composition, either before or after dilution or both, is suitable for nebulization and inhalation. In some embodiments, the average particle size of the nanoparticles in the nanoparticle (e.g., nano-emulsion) composition is determined by dynamic light scattering. In some embodiments, the zeta potential of the nanoparticles in the nanoparticle (e.g., nano-emulsion) composition is determined by zeta potential instrument.

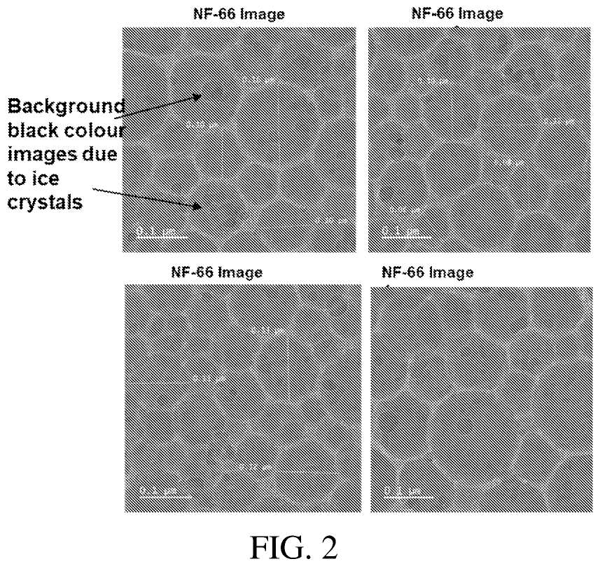

In some embodiments, the nanoparticles in the nanoparticle (e.g., nano-emulsion) composition have a multi lamellar structure comprising at least two lamellae layers. FIG. 2 shows CryoEM images for representative nanoparticles in the nanoparticle composition. In some embodiments, each lamellae layer comprises the perfluorocarbon sandwiched by two layers of the lipid.

In some embodiments, the nanoparticle (e.g., nano-emulsion) composition described herein is capable of dissolving oxygen. In some embodiments, the nanoparticle (e.g., nano-emulsion) composition is enriched with dissolved oxygen. In some embodiments, the nanoparticle (e.g., nano-emulsion) composition is enriched with dissolved oxygen at least about 1 ppm, for example, at least about any of 1 ppm, 2 ppm, 3 ppm, 4 ppm, 5 ppm, 6 ppm, 7 ppm, or 8 ppm. In some embodiments, the nanoparticle (e.g., nano-emulsion) composition is enriched with dissolved oxygen at about 1 ppm to about 20 ppm, for example, any of about 3 ppm to about 17 ppm, about 5 ppm to about 15 ppm, about 7 ppm to about 13 ppm, about 7 ppm to about 10 ppm, about 7 ppm, about 8 ppm, about 9 ppm, or about 10 ppm. In some embodiments, the concentration of the dissolved oxygen is determined by oxygen probe.

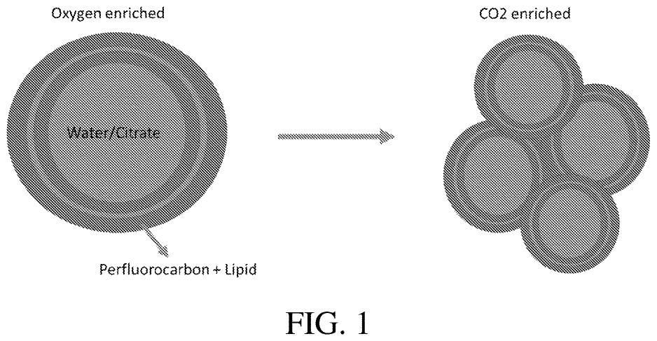

In some embodiments, the nanoparticle (e.g., nano-emulsion) composition described herein is capable of binding with CO₂. In some embodiments, nanoparticles in the nanoparticle (e.g., nano-emulsion) composition provided herein are capable of binding to O₂ and CO₂. In some embodiments, nanoparticles in the nanoparticle (e.g., nano-emulsion) composition provided herein are capable of selectively binding to O₂ or CO₂, depending on the concentration of 02 or CO₂, or the relative concentration between O₂ and CO₂. In some embodiments, as shown in FIG. 1, nanoparticles in the nanoparticle (e.g., nano-emulsion) composition provided herein are capable of facilitating CO₂ and O₂ exchange when administered to a tissue. In some embodiments, the tissue is in lung, at skin, in oral cavity, in colon, or in GI track. In some embodiments, nanoparticles in the nanoparticle (e.g., nano-emulsion) composition provided herein are capable of facilitating CO₂ and O₂ exchange in lung, as shown in FIG. 1, wherein before the nanoparticles arrive at alveolar epithelial cells, the nanoparticle (e.g., nano-emulsion) composition is enriched with O₂, and after contacting with the alveolar epithelial cells, the nanoparticle (e.g., nano-emulsion) composition is enriched with CO₂.

In some embodiments, the nanoparticle (e.g., nano-emulsion) composition is physical stable, for example, does not exhibit phase separation, under a temperature more than 10° C. (e.g., room temperature). In some embodiments, the nanoparticle (e.g., nano-emulsion) composition is physical stable at a temperature more than 10° C. (e.g., room temperature) for at least about 3 days, for example, at least about any of 4 days, 5 days, 6 days, 7 days, 8 days, 9 days, 10 days, or 12 days. In some embodiments, the nanoparticle (e.g., nano-emulsion) composition is chemically stable, for example, does not show oxidation or degradation, under a temperature more than 10° C. (e.g., room temperature). In some embodiments, the nanoparticle (e.g., nano-emulsion) composition is chemically stable at a temperature more than 10° C. (e.g., room temperature) for at least about 3 days, for example, at least about any of 4 days, 5 days, 6 days, 7 days, 8 days, 9 days, 10 days, or 12 days. In some embodiments, the nanoparticle (e.g., nano-emulsion) composition is stable for at least 10 days under room temperature without phase separation or chemical oxidation.

In some embodiments, the nanoparticle (e.g., nano-emulsion) composition comprises no more than 5% (w/w), for example, no more than any of 2% (w/w), 1% (w/w), 0.5% (w/w), 0.1% (w/w), or 0.01% (w/w), centrimonium bromide (CTAB). In some embodiments, the nanoparticle (e.g., nano-emulsion) composition is substantially free of centrimonium bromide (CTAB). CTAB is a quaternary ammonium surfactant and has known toxicology effects, such as respiratory tract irritation or skin irritation. In some embodiments, by getting rid of CTAB surfactant, the present nanoparticle (e.g., nano-emulsion) has reduced side effects while maintains the high stability.

In some embodiments, the nanoparticle (e.g., nano-emulsion) composition comprises no more than 5% (w/w), for example, no more than any of 2% (w/w), 1% (w/w), 0.5% (w/w), 0.1% (w/w), or 0.01% (w/w), Tween 80. In some embodiments, the nanoparticle (e.g., nano-emulsion) composition is substantially free of Tween 80. Tween 80 is a polyether, non-ionic surfactant and has known toxicology effects, such as respiratory tract irritation or skin irritation. In some embodiments, by getting rid of Tween 80, the present nanoparticle (e.g., nano-emulsion) has reduced side effects while maintains the high stability.

In some embodiments, the nanoparticle (e.g., nano-emulsion) composition comprises no more than 5% (w/w), for example, no more than any of 2% (w/w), 1% (w/w), 0.5% (w/w), 0.1% (w/w), or 0.01% (w/w), soya lecithin. In some embodiments, the nanoparticle (e.g., nano-emulsion) composition is substantially free of soya lecithin. Lecithin is a mixture of fats containing glycerolphospholipids and may have toxic effects when inhaled or applied to a wound, such as undesired irritation. In some embodiments, by getting rid of soya lecithin, the present nanoparticle (e.g., nano-emulsion) has reduced side effects while maintains the high stability.

In some embodiments, the nanoparticle (e.g., nano-emulsion) composition comprises no more than 5% (w/w), for example, no more than any of 2% (w/w), 1% (w/w), 0.5% (w/w), 0.1% (w/w), or 0.01% (w/w), polymeric surfactant. In some embodiments, the polymeric surfactant encompass poloxamer, and the nanoparticle (e.g., nano-emulsion) composition comprises no more than 5% (w/w), for example, no more than any of 2% (w/w), 1% (w/w), 0.5% (w/w), 0.1% (w/w), or 0.01% (w/w), poloxamer such as Pluronic®. Poloxamer are nonionic triblock copolymers composed of a central hydrophobic chain of polyoxypropylene (poly(propylene oxide)) flanked by two hydrophilic chains of polyoxyethylene (poly(ethylene oxide)). Poloxamer has been shown to have several toxicity when administered to animals, including causing hypercholesterolemia, hypertriglyceridemia, and tissue irritation. In some embodiments, by getting rid of poloxamer, the present nanoparticle (e.g., nano-emulsion) has reduced side effects while maintains the high stability. In some embodiments, by getting rid of polymeric surfactant, the present nanoparticle (e.g., nano-emulsion) has reduced side effects while maintains the high stability. In some embodiments, the nanoparticle (e.g., nano-emulsion) composition comprises no more than 5% (w/w), for example, no more than any of 2% (w/w), 1% (w/w), 0.5% (w/w), 0.1% (w/w), or 0.01% (w/w), any surfactant other than lipid.

In some embodiments, provided herein is a nanoparticle (e.g., nano-emulsion) composition prepared by mixing about 25%-40% (e.g., about 35.5%) (w/w) PFD, about 10% to 20% (e.g., about 14.5%) (w/w) PFTPA, about 0.1% to 1% (e.g. about 0.6%) (w/w) DPPC, about 1.5% to 2.5% (e.g., about 1.7%) (w/w) DSPC, and about 40% to 60% (e.g., about 47.7%) (w/w) pH 6.0 Citrate buffer (50 mM). In some embodiments of the foregoing, the mixing is carried out in a microfluidizer.

In some embodiments, the nanoparticle (e.g., nano-emulsion) composition is suitable for administration to a surface of a tissue. In some embodiments, the nanoparticle (e.g., nano-emulsion) composition is suitable for administration to the surface of pulmonary alveolus, optionally via inhalation. In some embodiments, the nanoparticle (e.g., nano-emulsion) composition is suitable for administration to the surface of skin, such as a wound or injury. In some embodiments, the nanoparticle (e.g., nano-emulsion) composition is suitable for administration to the surface of GI track, for example, via enema administration. In some embodiments, the nanoparticle (e.g., nano-emulsion) composition is administered in a non-invasive way. In some embodiments, the nanoparticle (e.g., nano-emulsion) composition is not administered via injection, including intravenous injection and intraperitoneal injection.

### Exemplary Nanoparticle (e.g., Nano-Emulsion) Compositions

In some embodiments, the nanoparticle (e.g., nano-emulsion) composition comprises:
(i) PFD at a weight percentage of about 25% to about 40% (e.g., about 35.5%) of the total nanoparticle (e.g., nano-emulsion) composition; (ii) PFTPA at a weight percentage of about 10% to 20% (e.g., about 14.5%) of the total nanoparticle (e.g., nano-emulsion) composition; (iii) DPPC at a weight percentage of about 0.1% to 1% (e.g. about 0.6%) of the total nanoparticle (e.g., nano-emulsion) composition; (iv) DSPC at a weight percentage of about 1.5% to 2.5% (e.g., about 1.7%) of the total nanoparticle (e.g., nano-emulsion) composition; and (v) pH 6.0 Citrate buffer (50 mM) at a weight percentage of 40% to 60% (e.g., about 47.7%) of the total nanoparticle (e.g., nano-emulsion) composition. In some embodiments, the nanoparticle (e.g., nano-emulsion) composition is contained in a nebulizer. In some embodiments, the nanoparticle (e.g., nano-emulsion) composition is comprised in a gel composition. In some embodiments, the nanoparticle (e.g., nano-emulsion) composition is comprised in a cream composition. In some embodiments, the nanoparticle (e.g., nano-emulsion) composition comprises no more than about 2% (w/w) polymeric surfactant of the total nanoparticle (e.g., nano-emulsion) composition. In some embodiments, the nanoparticle (e.g., nano-emulsion) composition has a viscosity of no more than about 100 cp. In some embodiments, the average particle size of the nanoparticles is no more than about 3 μm.

In some embodiments, the nanoparticle (e.g., nano-emulsion) composition comprises:
(i) PFD at a weight percentage of about 35% to about 55% (e.g., about 44.6%) of the total nanoparticle (e.g., nano-emulsion) composition; (ii) PFTPA at a weight percentage of about 10% to 20% (e.g., about 14.5%) of the total nanoparticle (e.g., nano-emulsion) composition; (iii) DPPC at a weight percentage of about 0.1% to 1% (e.g. about 0.6%) of the total nanoparticle (e.g., nano-emulsion) composition; (iv) DSPC at a weight percentage of about 1.5% to 2.5% (e.g., about 1.7%) of the total nanoparticle (e.g., nano-emulsion) composition; and (v) pH 6.0 Citrate buffer (50 mM) at a weight percentage of about 25% to about 45% (e.g., about 35.4%) of the total nanoparticle (e.g., nano-emulsion) composition. In some embodiments, the nanoparticle (e.g., nano-emulsion) composition is contained in a nebulizer. In some embodiments, the nanoparticle (e.g., nano-emulsion) composition is comprised in a gel composition. In some embodiments, the nanoparticle (e.g., nano-emulsion) composition is comprised in a cream composition. In some embodiments, the nanoparticle (e.g., nano-emulsion) composition comprises no more than about 2% (w/w) polymeric surfactant of the total nanoparticle (e.g., nano-emulsion) composition. In some embodiments, the nanoparticle (e.g., nano-emulsion) composition has a viscosity of no more than about 100 cp. In some embodiments, the average particle size of the nanoparticles is no more than about 3 μm.

In some embodiments, the nanoparticle (e.g., nano-emulsion) composition comprises:
(i) PFD at a weight percentage of about 40% to about 60% (e.g. about 50%) of the total nanoparticle (e.g., nano-emulsion) composition; (ii) PFTPA at a weight percentage of about 10% to about 30% (e.g., about 20%) of the total nanoparticle (e.g., nano-emulsion) composition; (iii) DPPC at a weight percentage of 0.1% to 1% (e.g. about 0.7%) of the total nanoparticle (e.g., nano-emulsion) composition; (iv) DSPC at a weight percentage of about 1% to 3% (e.g. about 2%) of the total nanoparticle (e.g., nano-emulsion) composition; and (v) pH 6.8 Phosphate buffer at a weight percentage of about 15% to about 40% (e.g. about 27.3%) of the total nanoparticle (e.g., nano-emulsion) composition. In some embodiments, the nanoparticle (e.g., nano-emulsion) composition is contained in a nebulizer. In some embodiments, the nanoparticle (e.g., nano-emulsion) composition is comprised in a gel composition. In some embodiments, the nanoparticle (e.g., nano-emulsion) composition is comprised in a cream composition. In some embodiments, the nanoparticle (e.g., nano-emulsion) composition comprises no more than about 2% (w/w) polymeric surfactant of the total nanoparticle (e.g., nano-emulsion) composition. In some embodiments, the nanoparticle (e.g., nano-emulsion) composition has a viscosity of no more than about 100 cp. In some embodiments, the average particle size of the nanoparticles is no more than about 3 μm.

In some embodiments, the nanoparticle (e.g., nano-emulsion) composition comprises:
(i) PFD at a weight percentage of about 25% to about 40% (e.g., about 30% to 40%, or about 35.5%) of the total nanoparticle composition;
(ii) PFTPA at a weight percentage of about 10% to 20% (e.g., about 12% to 18%, or about 14.5%) of the total nanoparticle composition;
(iii) DPPC at a weight percentage of about 0.1% to 1% (e.g. about 0.2% to 0.8%, or about 0.6%) of the total nanoparticle (e.g., nano-emulsion) composition;
(iv) DSPC at a weight percentage of about 1.5% to 2.5% (e.g., about 1.5% to about 2%, or about 1.7%) of the total nanoparticle composition;
(v) citric acid at a weight percentage of about 0.01% to 0.15% (e.g., about 0.05% to 0.15%, or about 0.1%) of the total nanoparticle composition;
(vi) sodium citrate dihydrate at a weight percentage of about 0.5% to about 2% (e.g., about 0.5% to about 1.5%, or about 1.2%) of the total nanoparticle composition; and
(viii) water at a weight percentage of about 30% to about 60% (e.g., about 40% to about 50%, or about 46.4%) of the total nanoparticle composition.

In some embodiments, the nanoparticle (e.g., nano-emulsion) composition is contained in a nebulizer. In some embodiments, the nanoparticle (e.g., nano-emulsion) composition is comprised in a gel composition. In some embodiments, the nanoparticle (e.g., nano-emulsion) composition is comprised in a cream composition. In some embodiments, the nanoparticle (e.g., nano-emulsion) composition comprises no more than about 2% (w/w) polymeric surfactant of the total nanoparticle (e.g., nano-emulsion) composition. In some embodiments, the nanoparticle (e.g., nano-emulsion) composition has a viscosity of no more than about 100 cp. In some embodiments, the average particle size of the nanoparticles is no more than about 3 μm.

In some embodiments, the nanoparticle (e.g., nano-emulsion) composition comprises:
(i) a perfluorocarbon component comprising PFD and PFTPA at a weight ratio of about 2:1 to about 3:1 (e.g., about 2:1 to about 2.6:1, or about 2.4:1);
(ii) a lipid component comprising DSPC and DPPC at a weight ratio of about 1:1 to about 5:1 (e.g., about 2:1 to about 3.5:1, or about 2.8:1);
(iii) a buffer component comprising sodium citrate hydrate and citric acid at a weight ratio of about 5:1 to about 20:1 (e.g., about 7:1 to about 15:1, or about 12:1); wherein
the weight ratio between the perfluorocarbon component and the lipid component is about 10:1 to about 30:1 (e.g., about 15:1 to about 23:1, or about 23:1); and
the weight ratio between the perfluorocarbon component and the buffer component is about 2.5:1 to about 5 (e.g., about 3.5:1 to about 4.5:1, or about 3.8:1).

In some embodiments, the nanoparticle (e.g., nano-emulsion) composition is contained in a nebulizer. In some embodiments, the nanoparticle (e.g., nano-emulsion) composition is comprised in a gel composition. In some embodiments, the nanoparticle (e.g., nano-emulsion) composition is comprised in a cream composition. In some embodiments, the nanoparticle (e.g., nano-emulsion) composition comprises no more than about 2% (w/w) polymeric surfactant. In some embodiments, the nanoparticle (e.g., nano-emulsion) composition has a viscosity of no more than about 100 cp. In some embodiments, the average particle size of the nanoparticles is no more than about 3 μm.

In some embodiments, any of the foregoing nanoparticle (e.g., nano-emulsion) compositions can be used as an inhalable formulation. In some embodiments, any of the foregoing nanoparticle (e.g., nano-emulsion) compositions can be mixed together with an pharmaceutically acceptable excipient to prepare for a wound healing composition, an enema gel, or a topical cream, as detailed herein.

### Method of Preparation

In certain aspects, provided are methods of preparing the nanoparticle compositions described herein.

Generally, in some embodiments, the methods to make the nanoparticle compositions described herein comprise admixing lipid and perfluorocarbon components in a solvent, such as a water miscible solvent, e.g., ethanol, subjecting the mixture to a high shear condition, and removing the solvent. In some embodiments, the lipid component is first admixed in a solvent and then the perfluorocarbon component is added. In some embodiments, the solvent is an organic solvent such as a ketone, ester, ether, chlorinated solvent, and other solvent known in the art. In some embodiments, the high shear condition is provided by one of more of sonication, homogenization, or microfluidization.

Techniques for subjecting a mixture to a high shear condition are known in the art. In some embodiments, the high shear condition is provided sonication, including by probe sonication or ultrasonication, homogenization, or microfluidization. For example, in some embodiments, the mixture is subjected to high pressure homogenization (e.g., using an Avestin, APV Gaulin, Microfluidizer™ such as a Microfluidizer™ Processor M-1 10EH from Microfluidics, Stansted, or Ultra Turrax homogenizer). In some embodiments, subjecting a mixture to a high shear condition comprises one or more cycles of a high shear condition. For example, a mixture may be cycled through a high pressure homogenizer for between about 2 to about 100 cycles, such as about 5 to about 50 cycles or about 8 to about 20 cycles (e.g., about any one of 8, 10, 12, 14, 16, 18 or 20 cycles). Solvent, such as ethanol, can be removed by evaporation utilizing suitable equipment known for this purpose, including, but not limited to, rotary evaporators, falling film evaporators, wiped film evaporators, spray driers, and the like that can be operated in batch mode or in continuous operation. In some embodiments, the solvent may be removed by exposure to the atmosphere or at reduced pressure (such as at about any one of 25 mm Hg, 30 mm Hg, 40 mm Hg, 50 mm Hg, 100 mm Hg, 200 mm Hg, or 300 mm Hg). The amount of time used to remove the solvent may be adjusted based on the volume of the formulation and the amount of solvent present.

In one aspect, provided herein is a method of preparing a nanoparticle (e.g., nano-emulsion) composition comprising nanoparticles, wherein the nanoparticles comprise a perfluorocarbon component and a lipid component, the method comprising (i) mixing a lipid with a solvent; (ii) evaporating the solvent in the mixture of (i); and (iii) adding a perfluorocarbon to the mixture of (ii). In some embodiments, the solvent is an alcohol. In some embodiments, the solvent is ethanol. In some embodiments, the solvent is evaporated by rotatory evaporation. In some embodiments, the mixture of (iii) is kept on sonication for a period of time, optionally about 30 minutes to about 1 hour.

In one aspect, provided herein is a method of preparing a nanoparticle (e.g., nano-emulsion) composition comprising nanoparticles, wherein the nanoparticles comprise a perfluorocarbon component and a lipid component, the method comprising (i) mixing a lipid with a solvent; (ii) adding a perfluorocarbon to the mixture of (i); (iii) homogenizing the mixture of (ii); and (iv) evaporating the solvent. In some embodiments, the solvent is a water miscible solvent, such as an alcohol. In some embodiments, the solvent is ethanol. In some embodiments, the solvent is evaporated by rotatory evaporation. In some embodiments, in step (i), the lipid is mixed with the solvent together with a surfactant. In some embodiments, in step (i), the lipid is mixed with the solvent without a surfactant. In some embodiments, the method further comprises (v) dispersing the mixture of (iv) in water or a buffer to form a nanoparticle composition, optionally under probe sonication.

In one aspect, provided herein is a method of preparing a nanoparticle (e.g., nano-emulsion) composition comprising nanoparticles, wherein the nanoparticles comprise a perfluorocarbon component and a lipid component, the method comprising (i) combining a lipid with a perfluorocarbon to form a mixture; (ii) homogenizing the mixture. In some embodiments, the homogenization is done by probe homogenization. In some embodiments, the method further comprises (iii) adding water or buffer to the mixture to obtain a second mixture; and (iv) homogenizing the second mixture. In some embodiments, the homogenization may be carried out at about 1 rpm to about 5 rpm, such as about 3.4 rpm. In some embodiments, the second mixture in (iv) may be homogenized for at least 1 minute, such as about any of 1 minute to 60 minutes, 10 minutes to 40 minutes, 10 minutes to 30 minutes, or about 20 minutes.

In one aspect, provided herein is a method of preparing a nanoparticle (e.g., nano-emulsion) composition comprising nanoparticles, wherein the nanoparticles comprise a perfluorocarbon component and a lipid component, the method comprising (i) adding a lipid in a solvent to form a first mixture; (ii) adding a perfluorocarbon to the first mixture to form a second mixture; and (iii) probe sonicating the second mixture of. In some embodiments, in step (i), the lipid is added to the solvent together with about 30% to 70% (e.g., about 50%) of the surfactant needed in the composition. In some embodiments, the first mixture is clear. In some embodiments, the solvent is a water miscible solvent, such as an alcohol. In some embodiments, the solvent is ethanol. In some embodiments, the second mixture is probe sonicated for at least about 1 minute, such as about any of 1 minute to 30 minutes, 5 minutes to 20 minutes, or about 10 minutes. In some embodiments, the method further comprises adding an aqueous solution to the first mixture, wherein the aqueous solution comprises a surfactant. In some embodiments, the method further comprises dispersing the probe sonicated second mixture in an aqueous surfactant solution. In some embodiments, the surfactant (e.g., non-lipid or polymeric surfactant) is about 30% to 70% (e.g., about 50%) of the surfactant needed in the composition.

In one aspect, provided herein is a method of preparing a nanoparticle (e.g., nano-emulsion) composition comprising nanoparticles, wherein the nanoparticles comprise a perfluorocarbon component and a lipid component, the method comprising (i) mixing a lipid with a solvent; (ii) adding a perfluorocarbon to the mixture of (i); (iii) probe sonicating the mixture of (ii); and (iv) slowly adding water or a buffer to the mixture of (iii). In some embodiments, the solvent is a water miscible solvent, such as an alcohol. In some embodiments, the solvent is ethanol. In some embodiments, in step (iv), an aqueous solution comprises a surfactant and optionally a buffer is added to the mixture of (iii). In some embodiments, the method further comprises probe sonicating the mixture after step (iv). In some embodiments, the mixture of (iv) is probe sonicated for about 30 minutes to about 1 hour.

In one aspect, provided herein is a method of preparing a nanoparticle (e.g., nano-emulsion) composition comprising nanoparticles, wherein the nanoparticles comprise a perfluorocarbon component and a lipid component, the method comprising (i) mixing a lipid with a solvent; (ii) evaporating the solvent in the mixture of (i) to form a thin film; (iii) hydrating the thin film by adding water or a buffer; and (iv) adding a perfluorocarbon to the mixture of (iii). In some embodiments, the solvent is a water miscible solvent, such as an alcohol. In some embodiments, the solvent is ethanol. In some embodiments, the solvent is evaporated by rotatory evaporation. In some embodiments, in step (iii), the thin film is hydrated by an aqueous solution comprises a surfactant and optionally a buffer. In some embodiments, the mixture of (iv) is kept on sonication (e.g., probe sonication) for a period of time, optionally about 5 minutes to about 30 minutes.

In some embodiments, provided herein is a method of preparing a nanoparticle (e.g., nano-emulsion) composition comprising nanoparticles, wherein the nanoparticles comprise a perfluorocarbon component and a lipid component, the method comprising (i) mixing a lipid with a perfluorocarbon; (ii) slowly adding water or a buffer to the mixture of (i); and (iii) microfluidizing the mixture of (ii). In some embodiments of (ii), a citrate buffer is added to the mixture of (i). In some embodiments, step (i) may be done via probe homogenization. In some embodiments, step (ii) may be done via probe homogenization. In some embodiments of step (ii), an aqueous solution comprises a surfactant and optionally a buffer is added to the mixture of (i). In some embodiments, the aqueous solution does not comprise (e.g., comprises less than 5 wt. %, 4 wt. %, 3 wt. %, 2 wt. %, 1 wt. %, or 0.1 wt. %) a surfactant. In some embodiments, the mixture of (ii) is microfluidized at about 10000 PSI to about 30000 PSI, optionally at about 20000 PSI or 25000 PSI. In some embodiments, the mixture of (ii) is microfluidized for at least 2 cycles, for example, 2 cycles, 3 cycles, 4 cycles, or 5 cycles. In some embodiments, the composition is cooled between each microfluidization cycle. In some embodiments, the composition is further sterilized by autoclave. In some embodiments, the composition is sterilized at a temperature of at least 100° C., such as about any of 105° C., 110° C., 115° C., or 120° C.

In some embodiments, the lipid comprises DSPC and DPPC. In some embodiments, the lipid is a mixture of DSPC and DPPC. In some embodiments, the perfluorocarbon comprises PFD and PFTA. In some embodiments, the perfluorocarbon is a mixture of PFD and PFTA.

In certain aspects, provided are nanoparticle compositions produced using the methods taught herein. For example, in some embodiments, provided is a nanoparticle composition produced by admixing lipid and perfluorocarbon components in a solvent, such as a water miscible solvent, e.g., ethanol, subjecting the mixture to a high shear condition, and removing the solvent to produce the nanoparticle composition.

### III. Topical Formulation

### Inhalable Formulation

In one aspect, provided herein is an inhalable formulation comprising the nanoparticle (e.g., nano-emulsion) composition described herein. In some embodiments, the nanoparticle (e.g., nano-emulsion) composition described herein is a composition suitable for direct nebulization and inhalation. In some embodiments, the nanoparticle (e.g., nano-emulsion) composition described herein is an inhalable formulation.

In some embodiments, the inhalable formulation comprising the nanoparticle (e.g., nano-emulsion) composition is capable of binding to O₂. In some embodiments, the inhalable formulation comprising the nanoparticle (e.g., nano-emulsion) composition is capable of binding to CO₂. In some embodiments, the inhalable formulation comprising the nanoparticle (e.g., nano-emulsion) composition is capable of selectively binding to O₂ or CO₂, depending on the concentration of O₂ or CO₂, or the relative concentration between O₂ and CO₂. In some embodiments, before the nanoparticles arrive at alveolar epithelial cells, the nanoparticle (e.g., nano-emulsion) composition is enriched with O₂, and after contacting with the alveolar epithelial cells, the nanoparticle (e.g., nano-emulsion) composition is enriched with CO₂. In some embodiments, the inhalable formulation comprising the nanoparticle (e.g., nano-emulsion) composition can be exhaled. In some embodiments, the inhalable formulation comprising the nanoparticle (e.g., nano-emulsion) composition is capable of delivering O₂ to the lung.

Also provided herein are devices (such as a nebulizer) comprising the inhalable formulation useful for administering the inhalable formulation.

### Wound Healing Composition

In one aspect, provided herein is a wound healing composition comprising the nanoparticle (e.g., nano-emulsion) composition described herein and a pharmaceutically acceptable excipient

In some embodiments, the wound healing composition comprises the nanoparticle (e.g., nano-emulsion) composition described herein, silver dioxide, and a collagen. In some embodiments, the collagen is a bovine collagen. In some embodiments, the bovine collagen is a hydrolyzed type 1 bovine collagen or a porous type 1 bovine collagen. In some embodiments, the hydrolyzed type 1 bovine collagen comprises 10% collagen content processed to have fetal skin-like collagen structures. In some embodiments, the porous type 1 bovine collagen is processed to have fetal skin-like collagen structures. In some embodiments, the collagen is a human collagen.

In some embodiments, the nanoparticle (e.g., nano-emulsion) composition is about 30% to about 90% (w/w), such as about any of 40% to 90% (w/w), 40% to 85% (w/w), 50% to 85% (w/w), 55% (w/w), or 82% (w/w), of the total wound healing composition.

In some embodiments, the silver dioxide is about 0.1% to about 5% (w/w), such as about any of 0.5% to 3% (w/w), 0.5% to 2.5% (w/w), 0.5% to 2% (w/w), 1% (w/w), or 1.5% (w/w), of the total wound healing composition.

In some embodiments, the collagen (e.g., bovine collagen) is about 10% to about 65% (w/w), such as about any of 10% to 60% (w/w), 10% to 55% (w/w), 10% to 50% (w/w), 15% to 50% (w/w), 15% (w/w), or 44% (w/w), of the total wound healing composition. In some embodiments, the collagen is a porous type 1 bovine collagen at a weight percentage of about 15% of the total wound healing composition. In some embodiments, the collagen is a hydrolyzed type 1 bovine collagen at a weight percentage of about 44% of the total wound healing composition.

In some embodiments, the wound healing composition comprises:
(a) a nanoparticle (e.g., nano-emulsion) composition at a weight percentage of about 40% to about 70% (e.g., about 55%) of the total wound healing composition, comprising
(i) PFD at a weight percentage of about 25% to about 40% (e.g., about 35.5%) of the total nanoparticle (e.g., nano-emulsion) composition;
(ii) PFTPA at a weight percentage of about 10% to 20% (e.g., about 14.5%) of the total nanoparticle (e.g., nano-emulsion) composition;
(iii) DPPC at a weight percentage of about 0.1% to 1% (e.g. about 0.6%) of the total nanoparticle (e.g., nano-emulsion) composition;
(iv) DSPC at a weight percentage of about 1.5% to 2.5% (e.g., about 1.7%) of the total nanoparticle (e.g., nano-emulsion) composition; and
(v) pH 6.0 Citrate buffer (50 mM) at a weight percentage of about 40% to 60% (e.g., about 47.7%) of the total nanoparticle (e.g., nano-emulsion) composition;
(b) silver dioxide at a weight percentage of about 0.5% to about 1.5% (e.g., about 1%) of the total wound healing composition, and
(c) a hydrolyzed type 1 bovine collagen at a weight percentage of about 35% to about 55% (e.g., about 44%) of the total wound healing composition, wherein the hydrolyzed type 1 bovine collagen comprises about 10% collagen content processed to have fetal skin-like collagen structures.

In some embodiments, the wound healing composition comprises:
(a) a nanoparticle (e.g., nano-emulsion) composition at a weight percentage of about 70% to about 90% (e.g., about 82%) of the total wound healing composition, comprising
(i) PFDS at a weight percentage of about 25% to about 40% (e.g., about 35.5%) of the total nanoparticle (e.g., nano-emulsion) composition;
(ii) PFTPA at a weight percentage of about 10% to 20% (e.g., about 14.5%) of the total nanoparticle (e.g., nano-emulsion) composition;
(iii) DPPC at a weight percentage of about 0.1% to 1% (e.g. about 0.6%) of the total nanoparticle (e.g., nano-emulsion) composition;
(iv) DSPC at a weight percentage of about 1.5% to 2.5% (e.g., about 1.7%) of the total nanoparticle (e.g., nano-emulsion) composition; and
(v) pH 6.0 Citrate buffer (50 mM) at a weight percentage of about 40% to 60% (e.g., about 47.7%) of the total nanoparticle (e.g., nano-emulsion) composition;
(b) silver dioxide at a weight percentage of about 1% to about 2% (e.g., 1.5%) of the total wound healing composition, and
(c) a porous type 1 bovine collagen at a weight percentage of about 10% to about 20% (e.g., about 15%) of the total wound healing composition, wherein the porous type 1 bovine collagen is processed to have fetal skin-like collagen structures.

In some embodiments, the wound healing composition comprises:
(a) a nanoparticle (e.g., nano-emulsion) composition at a weight percentage of about 70% to about 90% (e.g., about 82%) of the total wound healing composition, comprising
(i) PFD at a weight percentage of about 25% to about 40% (e.g., about 30% to 40%, or about 35.5%) of the total nanoparticle composition;
(ii) PFTPA at a weight percentage of about 10% to 20% (e.g., about 12% to 18%, or about 14.5%) of the total nanoparticle composition;
(iii) DPPC at a weight percentage of about 0.1% to 1% (e.g. about 0.2% to 0.8%, or about 0.6%) of the total nanoparticle (e.g., nano-emulsion) composition;
(iv) DSPC at a weight percentage of about 1.5% to 2.5% (e.g., about 1.5% to about 2%, or about 1.7%) of the total nanoparticle composition;
(v) citric acid at a weight percentage of about 0.01% to 0.15% (e.g., about 0.05% to 0.15%, or about 0.1%) of the total nanoparticle composition;
(vi) sodium citrate dihydrate at a weight percentage of about 0.5% to about 2% (e.g., about 0.5% to about 1.5%, or about 1.2%) of the total nanoparticle composition; and
(viii) water at a weight percentage of about 30% to about 60% (e.g., about 40% to about 50%, or about 46.4%) of the total nanoparticle composition;
(b) silver dioxide at a weight percentage of about 1% to about 2% (e.g., 1.5%) of the total wound healing composition, and
(c) a porous type 1 bovine collagen at a weight percentage of about 10% to about 20% (e.g., about 15%) of the total wound healing composition, wherein the porous type 1 bovine collagen is processed to have fetal skin-like collagen structures.

In some embodiments, the wound healing composition comprises:
(a) a nanoparticle (e.g., nano-emulsion) composition at a weight percentage of about 70% to about 90% (e.g., about 82%) of the total wound healing composition, comprising
(i) a perfluorocarbon component comprising PFD and PFTPA at a weight ratio of about 2:1 to about 3:1 (e.g., about 2:1 to about 2.6:1, or about 2.4:1);
(ii) a lipid component comprising DSPC and DPPC at a weight ratio of about 1:1 to about 5:1 (e.g., about 2:1 to about 3.5:1, or about 2.8:1); and
(iii) a buffer component comprising sodium citrate hydrate and citric acid at a weight ratio of about 5:1 to about 20:1 (e.g., about 7:1 to about 15:1, such as about 12:1); wherein
the weight ratio between the perfluorocarbon component and the lipid component in the nanoparticle composition is about 10:1 to about 30:1 (e.g., about 15:1 to about 23:1, or about 23:1); and
the weight ratio between the perfluorocarbon component and the buffer component in the nanoparticle composition is about 2.5:1 to about 5 (e.g., about 3.5:1 to about 4.5:1, or about 3.8:1);
(b) silver dioxide at a weight percentage of about 1% to about 2% (e.g., 1.5%) of the total wound healing composition, and
(c) a porous type 1 bovine collagen at a weight percentage of about 10% to about 20% (e.g., about 15%) of the total wound healing composition, wherein the porous type 1 bovine collagen is processed to have fetal skin-like collagen structures.

In some embodiments, provided herein is a wound healing composition prepared by:
(1) preparing a nanoparticle composition, which occupies about 70% to about 90% (wt. %) (e.g., about 82%) of the total wound healing composition, wherein the nanoparticle composition is prepared by mixing:
(i) PFD at a weight percentage of about 25% to about 40% (e.g., about 30% to 40%, or about 35.5%) of the total nanoparticle composition;
(ii) PFTPA at a weight percentage of about 10% to 20% (e.g., about 12% to 18%, or about 14.5%) of the total nanoparticle composition;
(iii) DPPC at a weight percentage of about 0.1% to 1% (e.g. about 0.2% to 0.8%, or about 0.6%) of the total nanoparticle (e.g., nano-emulsion) composition;
(iv) DSPC at a weight percentage of about 1.5% to 2.5% (e.g., about 1.5% to about 2%, or about 1.7%) of the total nanoparticle composition;
(v) citric acid at a weight percentage of about 0.01% to 0.15% (e.g., about 0.05% to 0.15%, or about 0.1%) of the total nanoparticle composition;
(vi) sodium citrate dihydrate at a weight percentage of about 0.5% to about 2% (e.g., about 0.5% to about 1.5%, or about 1.2%) of the total nanoparticle composition; and
(viii) water at a weight percentage of about 30% to about 60% (e.g., about 40% to about 50%, or about 46.4%) of the total nanoparticle composition;
(2) adding silver dioxide to the nanoparticle composition, wherein the silver oxide occupies about 1% to about 2% (wt. %) (e.g., 1.5%) of the total wound healing composition, and
(3) adding a porous type 1 bovine collagen to the nanoparticle composition, wherein the collagen occupies about 10% to about 20% (wt. %) (e.g., about 15%) of the total wound healing composition to form the wound healing composition, and wherein the porous type 1 bovine collagen is processed to have fetal skin-like collagen structures.

In some embodiments, provided herein is a wound healing composition prepared by:
(1) preparing a nanoparticle composition, which occupies about 70% to about 90% (wt. %) (e.g., about 82%) of the total wound healing composition, wherein the nanoparticle composition is prepared by mixing
(i) a perfluorocarbon component comprising PFD and PFTPA at a weight ratio of about 2:1 to about 3:1 (e.g., about 2:1 to about 2.6:1, or about 2.4:1);
(ii) a lipid component comprising DSPC and DPPC at a weight ratio of about 1:1 to about 5:1 (e.g., about 2:1 to about 3.5:1, or about 2.8:1); and
(iii) a buffer component comprising sodium citrate hydrate and citric acid at a weight ratio of about 5:1 to about 20:1 (e.g., about 7:1 to about 15:1, such as about 12:1); wherein
the weight ratio between the perfluorocarbon component and the lipid component in the nanoparticle composition is about 10:1 to about 30:1 (e.g., about 15:1 to about 23:1, or about 23:1); and
the weight ratio between the perfluorocarbon component and the buffer component in the nanoparticle composition is about 2.5:1 to about 5 (e.g., about 3.5:1 to about 4.5:1, or about 3.8:1);
(2) adding silver dioxide to the nanoparticle composition, wherein the silver oxide occupies about 1% to about 2% (wt. %) (e.g., 1.5%) of the total wound healing composition, and
(3) adding a porous type 1 bovine collagen to the nanoparticle composition, wherein the collagen occupies about 10% to about 20% (wt. %) (e.g., about 15%) of the total wound healing composition to form the wound healing composition, and wherein the porous type 1 bovine collagen is processed to have fetal skin-like collagen structures.

### Enema Gel

In one aspect, provided herein is an enema gel comprising the nanoparticle (e.g., nano-emulsion) composition described herein and a pharmaceutically acceptable excipient. In some embodiments, the enema gel comprises the nanoparticle (e.g., nano-emulsion) composition described herein, a poloxamer, and a PBS solution.

In some embodiments, the nanoparticle (e.g., nano-emulsion) composition is about 20% to about 70% (w/w), such as about any of 20% to 60% (w/w), 25% to 55% (w/w), 25% to 50% (w/w), 35% to 50% (w/w), or 40% (w/w), of the total enema gel.

In some embodiments, the poloxamer is P407 poloxamer or P188 poloxamer. In some embodiments, the poloxamer is P407 poloxamer at a weight percentage of about 5% to about 50%, such as about any of 10% to 40%, 15% to 35%, or 17% to 25% of the total enema gel. In some embodiments, the poloxamer is P407 poloxamer at a weight percentage of about 25% of the total enema gel. In some embodiments, the PBS solution is about 35% of the total enema gel.

In some embodiments, the poloxamer is P188 poloxamer at a weight percentage of about 5% to about 70%, such as about any of 10% to 60%, 15% to 55%, or 20% to 50%, of the total enema gel. In some embodiments, the poloxamer is P188 poloxamer at a weight percentage of about 50% of the total enema gel. In some embodiments, the PBS solution is about 10% of the total enema gel.

In some embodiments, the enema gel comprises:
(a) a nanoparticle (e.g., nano-emulsion) composition at a weight percentage of about 30% to about 50% (e.g., about 40%) of the total enema gel, comprising
(i) PFD at a weight percentage of about 25% to about 40% (e.g., about 35.5%) of the total nanoparticle (e.g., nano-emulsion) composition;
(ii) PFTPA at a weight percentage of about 10% to 20% (e.g., about 14.5%) of the total nanoparticle (e.g., nano-emulsion) composition;
(iii) DPPC at a weight percentage of about 0.1% to 1% (e.g. about 0.6%) of the total nanoparticle (e.g., nano-emulsion) composition;
(iv) DSPC at a weight percentage of about 1.5% to 2.5% (e.g., about 1.7%) of the total nanoparticle (e.g., nano-emulsion) composition; and
(v) pH 6.0 Citrate buffer (50 mM) at a weight percentage of about 40% to 60% (e.g., about 47.7%) of the total nanoparticle (e.g., nano-emulsion) composition;
(b) P407 Poloxamer at a weight percentage of about 20% to about 30% (e.g., about 25%) of the total enema gel, and
(c) a PBS solution at a weight percentage of about 30% to about 40% (e.g., about 35%) of the total enema gel.

In some embodiments, the enema gel comprises:
(a) a nanoparticle (e.g., nano-emulsion) composition at a weight percentage of about 30% to about 50% (e.g., about 40%) of the total enema gel, comprising
(i) PFD at a weight percentage of about 25% to about 40% (e.g., about 35.5%) of the total nanoparticle (e.g., nano-emulsion) composition;
(ii) PFTPA at a weight percentage of about 10% to 20% (e.g., about 14.5%) of the total nanoparticle (e.g., nano-emulsion) composition;
(iii) DPPC at a weight percentage of about 0.1% to 1% (e.g. about 0.6%) of the total nanoparticle (e.g., nano-emulsion) composition;
(iv) DSPC at a weight percentage of about 1.5% to 2.5% (e.g., about 1.7%) of the total nanoparticle (e.g., nano-emulsion) composition; and
(v) pH 6.0 Citrate buffer (50 mM) at a weight percentage of about 40% to 60% (e.g., about 47.7%) of the total nanoparticle (e.g., nano-emulsion) composition;
(b) P188 Poloxamer at a weight percentage of about 40% to about 60% (e.g., about 50%) of the total enema gel, and
(c) a PBS solution at a weight percentage of about 5% to about 15% (e.g., about 10%) of the total enema gel.

In some embodiments, the enema gel comprises:
(a) a nanoparticle (e.g., nano-emulsion) composition at a weight percentage of about 70% to about 90% (e.g., about 82%) of the total enema gel, comprising
(i) PFD at a weight percentage of about 25% to about 40% (e.g., about 30% to 40%, or about 35.5%) of the total nanoparticle composition;
(ii) PFTPA at a weight percentage of about 10% to 20% (e.g., about 12% to 18%, or about 14.5%) of the total nanoparticle composition;
(iii) DPPC at a weight percentage of about 0.1% to 1% (e.g. about 0.2% to 0.8%, or about 0.6%) of the total nanoparticle (e.g., nano-emulsion) composition;
(iv) DSPC at a weight percentage of about 1.5% to 2.5% (e.g., about 1.5% to about 2%, or about 1.7%) of the total nanoparticle composition;
(v) citric acid at a weight percentage of about 0.01% to 0.15% (e.g., about 0.05% to 0.15%, or about 0.1%) of the total nanoparticle composition;
(vi) sodium citrate dihydrate at a weight percentage of about 0.5% to about 2% (e.g., about 0.5% to about 1.5%, or about 1.2%) of the total nanoparticle composition; and
(viii) water at a weight percentage of about 30% to about 60% (e.g., about 40% to about 50%, or about 46.4%) of the total nanoparticle composition;
(b) P407 Poloxamer at a weight percentage of about 20% to about 30% (e.g., about 25%) of the total enema gel, and
(c) a PBS solution at a weight percentage of about 30% to about 40% (e.g., about 35%) of the total enema gel.

In some embodiments, the enema gel comprises:
(a) a nanoparticle (e.g., nano-emulsion) composition at a weight percentage of about 70% to about 90% (e.g., about 82%) of the total enema gel, comprising
(i) a perfluorocarbon component comprising PFD and PFTPA at a weight ratio of about 2:1 to about 3:1 (e.g., about 2:1 to about 2.6:1, or about 2.4:1);
(ii) a lipid component comprising DSPC and DPPC at a weight ratio of about 1:1 to about 5:1 (e.g., about 2:1 to about 3.5:1, or about 2.8:1); and
(iii) a buffer component comprising sodium citrate hydrate and citric acid at a weight ratio of about 5:1 to about 20:1 (e.g., about 7:1 to about 15:1, such as about 12:1); wherein
the weight ratio between the perfluorocarbon component and the lipid component in the nanoparticle composition is about 10:1 to about 30:1 (e.g., about 15:1 to about 23:1, or about 23:1); and
the weight ratio between the perfluorocarbon component and the buffer component in the nanoparticle composition is about 2.5:1 to about 5 (e.g., about 3.5:1 to about 4.5:1, or about 3.8:1);
(b) P188 Poloxamer at a weight percentage of about 40% to about 60% (e.g., about 50%) of the total enema gel, and
(c) a PBS solution at a weight percentage of about 5% to about 15% (e.g., about 10%) of the total enema gel.

In some embodiments, provided herein is an enema gel prepared by:
(1) preparing a nanoparticle composition, which occupies about 70% to about 90% (wt. %) (e.g., about 82%) of the total enema gel, wherein the nanoparticle composition is prepared by mixing
(i) PFD at a weight percentage of about 25% to about 40% (e.g., about 30% to 40%, or about 35.5%) of the total nanoparticle composition;
(ii) PFTPA at a weight percentage of about 10% to 20% (e.g., about 12% to 18%, or about 14.5%) of the total nanoparticle composition;
(iii) DPPC at a weight percentage of about 0.1% to 1% (e.g. about 0.2% to 0.8%, or about 0.6%) of the total nanoparticle (e.g., nano-emulsion) composition;
(iv) DSPC at a weight percentage of about 1.5% to 2.5% (e.g., about 1.5% to about 2%, or about 1.7%) of the total nanoparticle composition;
(v) citric acid at a weight percentage of about 0.01% to 0.15% (e.g., about 0.05% to 0.15%, or about 0.1%) of the total nanoparticle composition;
(vi) sodium citrate dihydrate at a weight percentage of about 0.5% to about 2% (e.g., about 0.5% to about 1.5%, or about 1.2%) of the total nanoparticle composition; and
(viii) water at a weight percentage of about 30% to about 60% (e.g., about 40% to about 50%, or about 46.4%) of the total nanoparticle composition;
(b) adding a P188 Poloxamer to the nanoparticle composition, wherein the P188 Poloxamer occupies a weight percentage of about 40% to about 60% (e.g., about 50%) of the total enema gel, and
(c) adding a PBS solution to the nanoparticle composition to form the enema gel, wherein the PBS solution occupies a weight percentage of about 5% to about 15% (e.g., about 10%) of the total enema gel.

In some embodiments, provided herein is an enema gel prepared by:
(1) preparing a nanoparticle composition, which occupies about 70% to about 90% (wt. %) (e.g., about 82%) of the total enema gel, wherein the nanoparticle composition is prepared by mixing
   (i) a perfluorocarbon component comprising PFD and PFTPA at a weight ratio of about 2:1 to about 3:1 (e.g., about 2:1 to about 2.6:1, or about 2.4:1);
   (ii) a lipid component comprising DSPC and DPPC at a weight ratio of about 1:1 to about 5:1 (e.g., about 2:1 to about 3.5:1, or about 2.8:1); and
   (iii) a buffer component comprising sodium citrate hydrate and citric acid at a weight ratio of about 5:1 to about 20:1 (e.g., about 7:1 to about 15:1, such as about 12:1); wherein
   the weight ratio between the perfluorocarbon component and the lipid component in the nanoparticle composition is about 10:1 to about 30:1 (e.g., about 15:1 to about 23:1, or about 23:1); and
   the weight ratio between the perfluorocarbon component and the buffer component in the nanoparticle composition is about 2.5:1 to about 5 (e.g., about 3.5:1 to about 4.5:1, or about 3.8:1);
   (b) adding a P188 Poloxamer to the nanoparticle composition, wherein the P188 Poloxamer occupies a weight percentage of about 40% to about 60% (e.g., about 50%) of the total enema gel, and
   (c) adding a PBS solution to the nanoparticle composition to form the enema gel, wherein the PBS solution occupies a weight percentage of about 5% to about 15% (e.g., about 10%) of the total enema gel.

### Topical Cream

In one aspect, provided herein is a cream composition comprising the nanoparticle (e.g., nano-emulsion) composition described herein and a pharmaceutically acceptable excipient. In some embodiments, the excipient comprises a thickening agent, an antioxidant, or a combination thereof.

In some embodiments, the nanoparticle (e.g., nano-emulsion) composition is presented at a weight percentage of about 20% to about 70% (w/w), such as about any of 20% to 70% (w/w), 25% to 55% (w/w), 25% to 50% (w/w), 40% to 60% (w/w), or 40% (w/w), of the total cream composition.

In some embodiments, the pharmaceutically acceptable excipient comprises a thickening agent. In some embodiments, the thickening agent comprises petroleum jelly. In some embodiments, the petroleum jelly is about 20% to about 80% (w/w), such as about any of 30% to 70% (w/w), 40% to 70% (w/w), 50% to 70% (w/w), 55% to 75% (w/w), or 60% (w/w), of the total cream composition. In some embodiments, the thickening agent comprises a colloidal oatmeal and a purified coconut oil. In some embodiments, the colloidal oatmeal is about 20% (w/w) to about 30% (w/w) of the total cream composition. In some embodiments, the purified coconut oil is about 5% to about 50%, such as about any of 10% to 40%, 15% to 35%, or 20% (w/w) to 30% (w/w) of the total cream composition.

In some embodiments, the pharmaceutically acceptable excipient comprises an antioxidant. In some embodiments, the antioxidant comprises a colloidal chaga extract, a Tinder Fungus extract, or a combination thereof. In some embodiments, the antioxidant comprises a colloidal chaga extract and the colloidal chaga extract is about 5% to about 50%, such as about any of 10% to 40%, 15% to 35%, or 20% (w/w) to 30% (w/w) of the total cream composition. In some embodiments, the antioxidant comprises a Tinder Fungus extract and the Tinder Fungus extract is about 5% to about 50%, such as about any of 10% to 40%, 15% to 35%, or 20% (w/w) to 30% (w/w) of the total cream composition.

In some embodiments, the cream composition comprises:
(a) a nanoparticle (e.g., nano-emulsion) composition at a weight percentage of about 40% of the total cream composition, comprising
   (i) PFD at a weight percentage of about 25% to about 40% (e.g., about 35.5%) of the total nanoparticle (e.g., nano-emulsion) composition;
   (ii) PFTPA at a weight percentage of about 10% to 20% (e.g., about 14.5%) of the total nanoparticle (e.g., nano-emulsion) composition;
   (iii) DPPC at a weight percentage of about 0.1% to 1% (e.g. about 0.6%) of the total nanoparticle (e.g., nano-emulsion) composition;
   (iv) DSPC at a weight percentage of about 1.5% to 2.5% (e.g., about 1.7%) of the total nanoparticle (e.g., nano-emulsion) composition; and
   (v) pH 6.0 Citrate buffer (50 mM) at a weight percentage of about 40% to 60% (e.g., about 47.7%) of the total nanoparticle (e.g., nano-emulsion) composition; and
(b) a petroleum jelly at a weight percentage of about 50% to about 70% (e.g., about 60%) of the total cream composition.

In some embodiments, the cream composition comprises:
(a) a nanoparticle (e.g., nano-emulsion) composition at a weight percentage of about 70% to about 90% (e.g., about 82%) of the total cream composition, comprising
   (i) PFD at a weight percentage of about 25% to about 40% (e.g., about 30% to 40%, or about 35.5%) of the total nanoparticle composition;
   (ii) PFTPA at a weight percentage of about 10% to 20% (e.g., about 12% to 18%, or about 14.5%) of the total nanoparticle composition;
   (iii) DPPC at a weight percentage of about 0.1% to 1% (e.g. about 0.2% to 0.8%, or about 0.6%) of the total nanoparticle (e.g., nano-emulsion) composition;
   (iv) DSPC at a weight percentage of about 1.5% to 2.5% (e.g., about 1.5% to about 2%, or about 1.7%) of the total nanoparticle composition;
   (v) citric acid at a weight percentage of about 0.01% to 0.15% (e.g., about 0.05% to 0.15%, or about 0.1%) of the total nanoparticle composition;
   (vi) sodium citrate dihydrate at a weight percentage of about 0.5% to about 2% (e.g., about 0.5% to about 1.5%, or about 1.2%) of the total nanoparticle composition; and
   (viii) water at a weight percentage of about 30% to about 60% (e.g., about 40% to about 50%, or about 46.4%) of the total nanoparticle composition; and
(b) a petroleum jelly at a weight percentage of about 50% to about 70% (e.g., about 60%) of the total cream composition.

In some embodiments, the cream composition comprises:
(a) a nanoparticle (e.g., nano-emulsion) composition at a weight percentage of about 70% to about 90% (e.g., about 82%) of the total cream composition, comprising
   (i) a perfluorocarbon component comprising PFD and PFTPA at a weight ratio of about 2:1 to about 3:1 (e.g., about 2:1 to about 2.6:1, or about 2.4:1);
   (ii) a lipid component comprising DSPC and DPPC at a weight ratio of about 1:1 to about 5:1 (e.g., about 2:1 to about 3.5:1, or about 2.8:1); and
   (iii) a buffer component comprising sodium citrate hydrate and citric acid at a weight ratio of about 5:1 to about 20:1 (e.g., about 7:1 to about 15:1, such as about 12:1); wherein
   the weight ratio between the perfluorocarbon component and the lipid component in the nanoparticle composition is about 10:1 to about 30:1 (e.g., about 15:1 to about 23:1, or about 23:1); and
   the weight ratio between the perfluorocarbon component and the buffer component in the nanoparticle composition is about 2.5:1 to about 5 (e.g., about 3.5:1 to about 4.5:1, or about 3.8:1); and
(b) a petroleum jelly at a weight percentage of about 50% to about 70% (e.g., about 60%) of the total cream composition.

In some embodiments, the cream composition comprises:
(a) a nanoparticle (e.g., nano-emulsion) composition at a weight percentage of about 40% to about 60% of the total cream composition, comprising
   (i) PFD at a weight percentage of about 25% to about 40% (e.g., about 35.5%) of the total nanoparticle (e.g., nano-emulsion) composition;
   (ii) PFTPA at a weight percentage of about 10% to 20% (e.g., about 14.5%) of the total nanoparticle (e.g., nano-emulsion) composition;
   (iii) DPPC at a weight percentage of about 0.1% to 1% (e.g. about 0.6%) of the total nanoparticle (e.g., nano-emulsion) composition;
   (iv) DSPC at a weight percentage of about 1.5% to 2.5% (e.g., about 1.7%) of the total nanoparticle (e.g., nano-emulsion) composition; and
   (v) pH 6.0 Citrate buffer (50 mM) at a weight percentage of about 40% to 60% (e.g., about 47.7%) of the total nanoparticle (e.g., nano-emulsion) composition;
(b) a Colloidal Oatmeal at a weight percentage of about 20% to about 30% of the total cream composition, and
(c) a purified coconut oil at a weight percentage of about 20% to about 30% of the total cream composition. structures.

In some embodiments, the cream composition comprises:
(a) a nanoparticle (e.g., nano-emulsion) composition at a weight percentage of about 40% to about 60% of the total cream composition, comprising
   (i) PFD at a weight percentage of about 25% to about 40% (e.g., about 30% to 40%, or about 35.5%) of the total nanoparticle composition;
   (ii) PFTPA at a weight percentage of about 10% to 20% (e.g., about 12% to 18%, or about 14.5%) of the total nanoparticle composition;
   (iii) DPPC at a weight percentage of about 0.1% to 1% (e.g. about 0.2% to 0.8%, or about 0.6%) of the total nanoparticle (e.g., nano-emulsion) composition;
   (iv) DSPC at a weight percentage of about 1.5% to 2.5% (e.g., about 1.5% to about 2%, or about 1.7%) of the total nanoparticle composition;
   (v) citric acid at a weight percentage of about 0.01% to 0.15% (e.g., about 0.05% to 0.15%, or about 0.1%) of the total nanoparticle composition;
   (vi) sodium citrate dihydrate at a weight percentage of about 0.5% to about 2% (e.g., about 0.5% to about 1.5%, or about 1.2%) of the total nanoparticle composition; and
   (viii) water at a weight percentage of about 30% to about 60% (e.g., about 40% to about 50%, or about 46.4%) of the total nanoparticle composition;
(b) a Colloidal Oatmeal at a weight percentage of about 20% to about 30% of the total cream composition, and
(c) a purified coconut oil at a weight percentage of about 20% to about 30% of the total cream composition. structures.

In some embodiments, the cream composition comprises:
(a) a nanoparticle (e.g., nano-emulsion) composition at a weight percentage of about 40% to about 60% of the total cream composition, comprising
   (i) a perfluorocarbon component comprising PFD and PFTPA at a weight ratio of about 2:1 to about 3:1 (e.g., about 2:1 to about 2.6:1, or about 2.4:1);
   (ii) a lipid component comprising DSPC and DPPC at a weight ratio of about 1:1 to about 5:1 (e.g., about 2:1 to about 3.5:1, or about 2.8:1); and
   (iii) a buffer component comprising sodium citrate hydrate and citric acid at a weight ratio of about 5:1 to about 20:1 (e.g., about 7:1 to about 15:1, such as about 12:1); wherein
   the weight ratio between the perfluorocarbon component and the lipid component in the nanoparticle composition is about 10:1 to about 30:1 (e.g., about 15:1 to about 23:1, or about 23:1); and
   the weight ratio between the perfluorocarbon component and the buffer component in the nanoparticle composition is about 2.5:1 to about 5 (e.g., about 3.5:1 to about 4.5:1, or about 3.8:1);
(b) a Colloidal Oatmeal at a weight percentage of about 20% to about 30% of the total cream composition, and
(c) a purified coconut oil at a weight percentage of about 20% to about 30% of the total cream composition. structures.

In some embodiments, the cream composition comprises:
(a) a nanoparticle (e.g., nano-emulsion) composition at a weight percentage of about 40% to about 60% of the total cream composition, comprising
   (i) PFD at a weight percentage of about 25% to about 40% (e.g., about 35.5%) of the total nanoparticle (e.g., nano-emulsion) composition;
   (ii) PFTPA at a weight percentage of about 10% to 20% (e.g., about 14.5%) of the total nanoparticle (e.g., nano-emulsion) composition;
   (iii) DPPC at a weight percentage of about 0.1% to 1% (e.g. about 0.6%) of the total nanoparticle (e.g., nano-emulsion) composition;
   (iv) DSPC at a weight percentage of about 1.5% to 2.5% (e.g., about 1.7%) of the total nanoparticle (e.g., nano-emulsion) composition; and
   (v) pH 6.0 Citrate buffer (50 mM) at a weight percentage of about 40% to 60% (e.g., about 47.7%) of the total nanoparticle (e.g., nano-emulsion) composition;
(b) a colloidal chaga extract at a weight percentage of about 20% to about 30% of the total cream composition, and
(c) a tinder fungus extract at a weight percentage of about 20% to about 30% of the total cream composition.

In some embodiments, the cream composition comprises:
(a) a nanoparticle (e.g., nano-emulsion) composition at a weight percentage of about 40% to about 60% of the total cream composition, comprising
   (i) PFD at a weight percentage of about 25% to about 40% (e.g., about 30% to 40%, or about 35.5%) of the total nanoparticle composition;
   (ii) PFTPA at a weight percentage of about 10% to 20% (e.g., about 12% to 18%, or about 14.5%) of the total nanoparticle composition;
   (iii) DPPC at a weight percentage of about 0.1% to 1% (e.g. about 0.2% to 0.8%, or about 0.6%) of the total nanoparticle (e.g., nano-emulsion) composition;
   (iv) DSPC at a weight percentage of about 1.5% to 2.5% (e.g., about 1.5% to about 2%, or about 1.7%) of the total nanoparticle composition;
   (v) citric acid at a weight percentage of about 0.01% to 0.15% (e.g., about 0.05% to 0.15%, or about 0.1%) of the total nanoparticle composition;
   (vi) sodium citrate dihydrate at a weight percentage of about 0.5% to about 2% (e.g., about 0.5% to about 1.5%, or about 1.2%) of the total nanoparticle composition; and
   (viii) water at a weight percentage of about 30% to about 60% (e.g., about 40% to about 50%, or about 46.4%) of the total nanoparticle composition;
(b) a colloidal chaga extract at a weight percentage of about 20% to about 30% of the total cream composition, and
(c) a tinder fungus extract at a weight percentage of about 20% to about 30% of the total cream composition.

In some embodiments, the cream composition comprises:
(a) a nanoparticle (e.g., nano-emulsion) composition at a weight percentage of about 40% to about 60% of the total cream composition, comprising
   (i) a perfluorocarbon component comprising PFD and PFTPA at a weight ratio of about 2:1 to about 3:1 (e.g., about 2:1 to about 2.6:1, or about 2.4:1);
   (ii) a lipid component comprising DSPC and DPPC at a weight ratio of about 1:1 to about 5:1 (e.g., about 2:1 to about 3.5:1, or about 2.8:1); and
   (iii) a buffer component comprising sodium citrate hydrate and citric acid at a weight ratio of about 5:1 to about 20:1 (e.g., about 7:1 to about 15:1, such as about 12:1); wherein
   the weight ratio between the perfluorocarbon component and the lipid component in the nanoparticle composition is about 10:1 to about 30:1 (e.g., about 15:1 to about 23:1, or about 23:1); and
   the weight ratio between the perfluorocarbon component and the buffer component in the nanoparticle composition is about 2.5:1 to about 5 (e.g., about 3.5:1 to about 4.5:1, or about 3.8:1);
(b) a colloidal chaga extract at a weight percentage of about 20% to about 30% of the total cream composition, and
(c) a tinder fungus extract at a weight percentage of about 20% to about 30% of the total cream composition.

### IV. Method of Treatment

In one aspect, provided herein is a method of use any of the compositions and formulations provided herein. In some embodiments, provided herein is a method of delivering oxygen to a tissue of a subject in need thereof, comprising administering (e.g., administering a pharmaceutically effective amount of) the nanoparticle (e.g., nano-emulsion) composition to the subject. In some embodiments, the tissue is a damaged tissue, for example a tissue that is in need of oxygen.

In some embodiments, the nanoparticle (e.g., nano-emulsion) composition is administered topically, i.e., to the surface of a tissue. In some embodiments, the advantages of the topical administration include, but are not limited to, the avoidance of the risks and inconvenience of parenteral treatment; the avoidance of the variable absorption and metabolism associated with intravenous or intraperitoneal injections; and the continuity of drug administration. In some embodiments, the suitable topical administration routes include, but are not limited to, buccal, cutaneous, endotracheal, enteral, nasal, nasogastric, rectal, respiratory (inhalation), sublingual, transdermal, or transmucosal. In some embodiments, the method provided herein comprises local administration of the nanoparticle (e.g., nano-emulsion) compositions described herein. In some embodiments, the local administration comprises topically administering the nanoparticle (e.g., nano-emulsion) compositions at a particular treatment site, (e.g., the digestive tract, the gastrointestinal (“GI”) tract, lung, eye, joint, or skin) so as to provide local administration to the area in need of treatment (e.g., oral cavity, GI tract, lung, colon, eye, or skin). In some embodiments, the nanoparticle (e.g., nano-emulsion) compositions can penetrate nails and can be directly and topically applied on nails. In certain embodiments, minimal systemic exposure of the chemical entity occurs during said local administration. In some embodiments, suitable local administration routes include, but are not limited to, rectal administration, sublingual administration, transmucosal administration, dermal administration, or inhalation. In some embodiments, the compositions are for other than intravenous or intraperitoneal injections.

In some embodiments, the nanoparticle (e.g., nano-emulsion) composition is administered in the form of the wound healing composition, the enema gel, or the cream composition provided herein. In some embodiments, the method comprises topically administering the nanoparticle (e.g., nano-emulsion) composition to the surface of a tissue. In some embodiments, the tissue is at skin. In some embodiments, the tissue is in the colon or GI. In some embodiments, the tissue is in lung.

In some embodiments, the nanoparticle (e.g., nano-emulsion) compositions, optionally in the form of the wound healing composition, the enema gel, or the cream composition provided herein, can be administered immediately as needed, such as in an emergency. In some embodiments, the nanoparticle (e.g., nano-emulsion) compositions, optionally in the form of the wound healing composition, the enema gel, or the cream composition provided herein, can be administered according to a suitable schedule, for example, about every day or about once a month. The dosages of the nanoparticle (e.g., nano-emulsion) compositions can be determined by those of ordinary skill in the art. Furthermore, it is noted that the clinician or treating physician will know how and when to interrupt, adjust, or terminate therapy in conjunction with the individual's response. In some embodiments, administration of the nanoparticle (e.g., nano-emulsion) compositions may be repeated until the condition to be treated gets alleviated or eliminated.

In some embodiments, the method treats or prevents hypoxemia in a subject in need thereof, comprising administering (e.g., administering a pharmaceutically effective amount of) the nanoparticle (e.g., nano-emulsion) composition to the subject. In some embodiments, provided herein is a method of treating or preventing a condition associated with hypoxemia in a subject in need thereof, comprising administering (e.g., administering a pharmaceutically effective amount of) the nanoparticle (e.g., nano-emulsion) composition to the subject. In some embodiments, provided herein is a method of alleviating or preventing symptoms associated with a condition associated with hypoxemia in a subject in need thereof, comprising administering (e.g., administering a pharmaceutically effective amount of) the nanoparticle (e.g., nano-emulsion) composition to the subject. In some embodiments, the condition includes, but are not limited to, acute respiratory distress syndrome (ARDS), acute lung injury, pulmonary fibrosis (idiopathic), mechanical ventilator induced lung injury, lung transplantation-induced acute graft dysfunction and bronchiolitis obliterans after lung transplantation, bronchial asthma, acute bronchitis, emphysema, chronic obstructive emphysema, chronic obstructive pulmonary disease, centrilobular emphysema, panacinar emphysema, chronic obstructive bronchitis, smoker's disease, reactive airway disease, cystic fibrosis, black lung disease, bronchiectasis, acquired bronchiectasis, kartaagener's syndrome, acelectasis, acute atelectasis, chronic acelectasis, pneumonia, essential thrombocythemia, legionnaire's disease, psittacosis, fibrogenic dust disease, hypersensitivity diseases of the lung, idiopathic infiltrative diseases of the lungs, chronic obstructive pulmonary disorder, adult respiratory distress syndrome, pulmonary tumors, pulmonary hypertension, and diseases caused by organic dust, cyanide poisoning, nicotine, insulin, irritant gases, Alzheimer's, nasal diseases like allergic rhinitis, sinusitis and chemicals like Cyanide, ozone, lung or sinus infections, cancer, sleep apnea, Migraines, COVID, chronic obstructive pulmonary disease (COPD), or asthma. In some embodiments, the condition is COVID, chronic obstructive pulmonary disease (COPD), asthma, or lung injury.

In some embodiments, provided herein is a method of treating a topical condition in a subject in need thereof, comprising administering the nanoparticle (e.g., nano-emulsion) composition described herein to the subject. In some embodiments, the nanoparticle (e.g., nano-emulsion) composition is administered in the form of the wound healing composition, the enema gel, or the cream composition provided herein. In some embodiments, the topical condition comprises a wound, an open injury, a burn, a colon or GI condition, or a pulmonary condition. In some embodiments, the pulmonary condition is COVID, chronic obstructive pulmonary disease (COPD), asthma, or lung injury. In some embodiments, the condition is ARDS. In some embodiments, the ARDS is caused by bacterial infection.

In some embodiments, provided herein is a method of treating a pulmonary condition in a subject in need thereof, comprising administering any of the nanoparticle compositions described herein to the subject. In some embodiments, the pulmonary condition is COVID, chronic obstructive pulmonary disease (COPD), asthma, or lung injury. In some embodiments, the pulmonary condition is acute respiratory distress (ARDS). In some embodiments, the ARDS is caused by bacterial infection. In some embodiments, the nanoparticle composition may be nebulized before being administered. In some embodiments, the nanoparticle composition is administered via inhalation.)

In some embodiments, provided herein is a method of treating a skin condition in a subject in need thereof, comprising administering any of the nanoparticle compositions, wound healing composition, or the cream described herein to the subject. In some embodiments, the skin condition is a wound, an open injury, or a burn. In some embodiments, the nanoparticle composition is administered topically, such as topically administered on skin. In some embodiments, the nanoparticle composition may be nebulized before being administered. In some embodiments, the nanoparticle composition is administered as a cream or aerosol.

In some embodiments, provided herein is a method of treating a GI condition in a subject in need thereof, comprising administering any of the nanoparticle compositions, or the enema gel described herein to the subject. In some embodiments, the GI condition is a GI track wound. In some embodiments, the nanoparticle composition is administered topically, such as topically administered on the GI track tissue surface.

In some embodiments, provided herein is a method of treating ARDS, comprising administering to the subject any of the nanoparticle composition provided herein. In some embodiments, the amount of nanoparticle composition administered is effective in delaying destruction of lung tissue in a subject, such as slowing down the decrease of intact alveoli in the subject by at least 5%, such as at least about any of 10%, 15%, 20%, 25%, 30%, 35%, 40%, 45%, 50%, 55%, 60%, 65%, 70%, 75%, 80%, 95%, 99%, or 100%. Hence in some embodiments, provided herein is a method of delaying destruction of lung tissue in a subject, comprising administering to the subject any of the nanoparticle composition provided herein. In some embodiments, the subject has ARDS. In some embodiments, the amount of nanoparticle composition administered is effective in reducing degradation of alveolar structures in the subject, such as reducing by at least 5%, such as at least about any of 10%, 15%, 20%, 25%, 30%, 35%, 40%, 45%, 50%, 55%, 60%, 65%, 70%, 75%, 80%, 95%, 99%, or 100%. Hence in some embodiments, provided herein is a method of reducing degradation of alveolar structures in a subject, comprising administering to the subject any of the nanoparticle composition provided herein. In some embodiments, the subject has ARDS. In some embodiments, the amount of nanoparticle composition administered is effective in increasing CFTR expression in the lung in the subject, such as increasing by at least 5%, such as at least about any of 10%, 15%, 20%, 25%, 30%, 35%, 40%, 45%, 50%, 55%, 60%, 65%, 70%, 75%, 80%, 95%, 99%, or 100%. In some embodiments, the amount of nanoparticle composition administered is effective in decreasing TRPV1 expression in a subject, such as decreasing by at least 5%, such as at least about any of 10%, 15%, 20%, 25%, 30%, 35%, 40%, 45%, 50%, 55%, 60%, 65%, 70%, 75%, 80%, 95%, 99%, or 100%. Hence in some embodiments, provided herein is a method of decreasing TRPV1 expression in a subject, comprising administering to the subject any of the nanoparticle composition provided herein. In some embodiments, the subject has ARDS. In some embodiments, the amount of nanoparticle composition administered is effective in decreasing COX-PTGS2 expression in a subject, such as decreasing by at least 5%, such as at least about any of 10%, 15%, 20%, 25%, 30%, 35%, 40%, 45%, 50%, 55%, 60%, 65%, 70%, 75%, 80%, 95%, 99%, or 100%. Hence in some embodiments, provided herein is a method of decreasing COX-PTGS2 expression in a subject, comprising administering to the subject any of the nanoparticle composition provided herein. In some embodiments, the subject has ARDS. In some embodiments, the nanoparticle composition comprises (i) a perfluorocarbon component comprising PFD and PFTPA at a weight ratio of about 2:1 to about 3:1 (e.g., about 2:1 to about 2.6:1, or about 2.4:1); (ii) a lipid component comprising DSPC and DPPC at a weight ratio of about 1:1 to about 5:1 (e.g., about 2:1 to about 3.5:1, or about 2.8:1); and (iii) a buffer component comprising sodium citrate hydrate and citric acid at a weight ratio of about 5:1 to about 20:1 (e.g., about 7:1 to about 15:1, such as about 12:1); wherein the weight ratio between the perfluorocarbon component and the lipid component in the nanoparticle composition is about 10:1 to about 30:1 (e.g., about 15:1 to about 23:1, or about 23:1); and the weight ratio between the perfluorocarbon component and the buffer component in the nanoparticle composition is about 2.5:1 to about 5 (e.g., about 3.5:1 to about 4.5:1, or about 3.8:1) . . . . In some embodiments, the nanoparticle composition is contained in a nebulizer. In some embodiments, the nanoparticle composition comprises no more than about 2% (w/w) polymeric surfactant of the total nanoparticle composition. In some embodiments, the nanoparticle composition has a viscosity of no more than about 100 cp. In some embodiments, the average particle size of the nanoparticles is no more than about 3 μm.

In some embodiments, provided herein is a method of restoring oxygenated Hemoglobin in a tissue of a subject, comprising administering to the subject any of the nanoparticle composition provided herein. In some embodiments, the nanoparticle composition is administered topically. In some embodiments, the nanoparticle composition can penetrate through skin. In some embodiments, the amount of the nanoparticle composition administered is effective in restoring at least 5% (e.g., increasing the amount of oxygenated Hemoglobin by at least about any of 10%, 15%, 20%, 25%, 30%, 35%, 40%, 45%, 50%, 55%, 60%, 65%, 70%, 75%, 80%, 95%, 99%, or 100%) oxygenated Hemoglobin in the tissue. In some embodiments, the amount of oxygenated Hemoglobin may be determined by oximetry, such as pulse oximetry. In some embodiments, the nanoparticle composition comprises (i) a perfluorocarbon component comprising PFD and PFTPA at a weight ratio of about 2:1 to about 3:1 (e.g., about 2:1 to about 2.6:1, or about 2.4:1); (ii) a lipid component comprising DSPC and DPPC at a weight ratio of about 1:1 to about 5:1 (e.g., about 2:1 to about 3.5:1, or about 2.8:1); and (iii) a buffer component comprising sodium citrate hydrate and citric acid at a weight ratio of about 5:1 to about 20:1 (e.g., about 7:1 to about 15:1, such as about 12:1); wherein the weight ratio between the perfluorocarbon component and the lipid component in the nanoparticle composition is about 10:1 to about 30:1 (e.g., about 15:1 to about 23:1, or about 23:1); and the weight ratio between the perfluorocarbon component and the buffer component in the nanoparticle composition is about 2.5:1 to about 5 (e.g., about 3.5:1 to about 4.5:1, or about 3.8:1) . . . . In some embodiments, the nanoparticle composition is contained in a nebulizer. In some embodiments, the nanoparticle composition is comprised in a gel composition. In some embodiments, the nanoparticle composition is comprised in a cream composition. In some embodiments, the nanoparticle composition comprises no more than about 2% (w/w) polymeric surfactant of the total nanoparticle composition. In some embodiments, the nanoparticle composition has a viscosity of no more than about 100 cp. In some embodiments, the average particle size of the nanoparticles is no more than about 3 μm.

### V. Kits

Also provided are articles of manufacture and/or kits containing any of the nanoparticle (e.g., nano-emulsion) composition described herein. In some embodiments, the articles of manufacture and/or kits contain any of the wound healing composition, the enema gel, or the cream composition provided herein. In some embodiments, the article of manufacture may comprise a container with a label. In some embodiments, the suitable containers include, for example, bottles, vials, and test tubes. The containers may be formed from a variety of materials such as glass or plastic. In some embodiments, the container may hold a nanoparticle (e.g., nano-emulsion) composition provided herein. In some embodiments, the container is a nebulizer. In some embodiments, the nebulizer contains the nanoparticle (e.g., nano-emulsion) composition described herein can nebulize the nanoparticle (e.g., nano-emulsion) composition before use. In some embodiments, the label on the container may indicate that the pharmaceutical composition is used for preventing, treating or suppressing a condition described herein, and may also indicate directions for use.

In one aspect, provided herein are kits containing any of the nanoparticle (e.g., nano-emulsion) composition described herein, and instructions for use. The kits may contain instructions for use in the treatment of any disease or condition described herein in an individual in need thereof. A kit may additionally contain any materials or equipment that may be used in the administration of the compound or composition, such as nebulizers.

### EXAMPLES

The presently disclosed subject matter will be better understood by reference to the following Examples, which are provided as exemplary of the invention, and not by way of limitation.

### Example 1. Method of Emulsion Preparation by Rotary Evaporation

### NF-1

1,2-Distearoyl-sn-glycero-3-phosphocholine (DSPC) and 1,2-Dipalmitoyl-sn-glycero-3-phosphocholine (DPPC) were added to ethanol, and the mixture was stirred until the solution became clear. The solution was then transferred into round-bottom flask (RBF) and subjected to rotary evaporation at 40° C. to evaporate the ethanol, and then kept overnight in vacuum oven to remove remaining amount of Ethanol at RT. PFD and PFTA were added to the film, kept on sonication for ~30 mins to obtain NF-1. The quantity of each component is summarized in Table 1.

### Example 2. Method of Emulsion Preparation by Homogenization

### NF-2, NF-3, NF-4

1,2-Distearoyl-sn-glycero-3-phosphocholine (DSPC) and 1,2-Dipalmitoyl-sn-glycero-3-phosphocholine (DPPC) were added to ethanol, and stirred until solution became clear. Then the mixture of PFD and PFTA were added slowly to above mixture and homogenized for 10 mins at 3.4 rpm. Upon homogenization, ethanol was removed from the mixture, and the vial containing lipid and perfluorocarbon mixture was redispersed with water to form NF2, NF-3, and NF-4. The quantity of each component is summarized in Table 1.

### Example 3. Method of Emulsion Preparation by Probe Sonication

### NF-5, NF-5A, NF-6, NF-7

1,2-Distearoyl-sn-glycero-3-phosphocholine (DSPC) and 1,2-Dipalmitoyl-sn-glycero-3-phosphocholine (DPPC) were added to ethanol, and the mixture was stirred until solution became clear. Part of the non-lipid surfactant (if any) was added, and the mixture was stirred until it became clear. This solution was added to the mixture of PFD and PFTA, and the mixture was probe sonicated for ~10 min at 40 amplitude. The above mixture was added slowly to water containing the remaining non-lipid surfactant (if any) upon probe sonication to form NF-5, NF-5A, NF-6, and NF-7. The quantity of each component is summarized in Table 1.

### NF-8, NF-9, NF-10

1,2-Distearoyl-sn-glycero-3-phosphocholine (DSPC) and 1,2-Dipalmitoyl-sn-glycero-3-phosphocholine (DPPC), and non-lipid surfactant (if any) were mixed with perfluorocarbon (PFD+PFTA) mixture, and the mixture was probe sonicated for about 5 mins. Water was added to above mixture slowly, and the new mixture was probe sonicated ~15 mins at 20 amplitude to afford NF-8, NF-9, and NF-10. The quantity of each component is summarized in Table 1.

### NF-11, NF-12, NF-13

1,2-Distearoyl-sn-glycero-3-phosphocholine (DSPC) and 1,2-Dipalmitoyl-sn-glycero-3-phosphocholine (DPPC), and non-lipid surfactant were mixed with API (PFD+PFTA) mixture, and the new mixture was probe sonicated for about 5 mins. Water was added to the above mixture slowly, and the mixture was probe sonicated for ~15 mins 20 amplitude to afford NF-11, NF-12, and NF-13. The quantity of each component is summarized in Table 1.

### Example 4. Method of Emulsion Preparation by Homogenization and Probe Sonication

### NF-14, NF-16, NF-17

1,2-Distearoyl-sn-glycero-3-phosphocholine (DSPC), 1,2-Dipalmitoyl-sn-glycero-3-phosphocholine (DPPC) and non-lipid surfactant (if any) was added to ethanol, and the mixture was stirred until solution became clear. Mixture of PFD and PFTA were then added slowly to the above mixture, and the resulting mixture was homogenized for 10 mins at 3.4 rpm. Upon homogenization, ethanol was removed from the mixture, and the remaining lipid and perfluorocarbon mixture was redispersed with water. The resulted solution was probe sonicated for ~15 mins 20 amplitude. The quantity of each component is summarized in Table 1.

### Example 5. Method of Emulsion Preparation by Homogenization

### NF-15

1,2-Distearoyl-sn-glycero-3-phosphocholine (DSPC), 1,2-Dipalmitoyl-sn-glycero-3-phosphocholine (DPPC) and non-lipid surfactant (if any) were added to ethanol, and the mixture was stirred until solution became clear. A mixture of PFD and PFTA were added slowly to above mixture upon homogenization. To the above mixture water was added on homogenization for 30 mins at 8.4 rpm. The resulted solution was kept at RT overnight from which ethanol was removed to afford NF-15. The quantity of each component is summarized in Table 1.

### Example 6. Method of Emulsion Preparation by Homogenization (e.g., Probe Homogenization) and Rota Evaporation

### NF-18

1,2-Distearoyl-sn-glycero-3-phosphocholine (DSPC), 1,2-Dipalmitoyl-sn-glycero-3-phosphocholine (DPPC) and non-lipid surfactant (if any) were added to ethanol, and the mixture was stirred until solution became clear. A mixture of PFD and PFTA were added slowly to above mixture upon homogenization. The above mixture was transferred into RBF for ethanol evaporation. The RBF containing lipid and perfluorocarbon mixture was redispersed with water, and the resulted solution was probe sonicated for ~10 mins 20 amplitude. The quantity of each component is summarized in Table 1.

### Example 7. Method of Emulsion Preparation by Direct Addition

### NF-19, NF-20, NF-24

1,2-Distearoyl-sn-glycero-3-phosphocholine (DSPC) and 1,2-Dipalmitoyl-sn-glycero-3-phosphocholine (DPPC), and non-lipid surfactant (if any) were mixed with perfluorocarbon (PFD+PFTA) mixture, and the resulted solution was probe sonicated for about 5 mins. Water was added to the above mixture slowly, and the new mixture was probe sonicated for about 15 mins at 40 amplitude to afford NF-19, NF-20, and NF-24. The quantity of each component is summarized in Table 1.

### Example 8. Method of Emulsion Preparation by Thin Film Hydration

### NF-21, NF-22, NF-23

1,2-Distearoyl-sn-glycero-3-phosphocholine (DSPC), 1,2-Dipalmitoyl-sn-glycero-3-phosphocholine (DPPC) and non-lipid surfactant (if any) were added to ethanol, and the mixture was stirred until became clear. The above solution was transferred into RBF and Rota evaporated, when a thin film was formed. The thin film was then hydrated with water. A mixture of PFD and PFTA were added slowly to above mixture on probe sonication at 20 amplitude 10 mins to afford NF-21, NF-22, and NF-23. The quantity of each component is summarized in Table 1.

### NF-25

1,2-Distearoyl-sn-glycero-3-phosphocholine (DSPC) and 1,2-Dipalmitoyl-sn-glycero-3-phosphocholine (DPPC), and HSPC were added to ethanol, and the solution was stirred until became clear. The above solution was transferred into a RBF, and Rota evaporated to form a thin film. The thin film was then hydrated with water. A mixture of PFD and PFTA was added slowly to the above mixture on probe sonication at 20 amplitude for 10 mins to afford NF-25. The quantity of each component is summarized in Table 1.

### NF-26, NF-27, NF-28, NF-29, NF-30

1,2-Dipalmitoyl-sn-glycero-3-phosphocholine (DPPC), 1,2-Distearoyl-sn-glycero-3-phosphocholine (DSPC) and non-lipid surfactant (e.g., hexadecyltrimethylammonium bromide (CTAB)) if any, were added to ethanol, and the solution was stirred until became clear. The above solution was transferred into RBF and Rota evaporated, when a thin film was formed. The thin film was then hydrated with water. A mixture of PFD and PFTA were added slowly to above mixture on probe sonication at 20 amplitude 10 mins to afford the titled compositions. The quantity of each component is summarized in Table 1.

### Example 9. Method of Emulsion Preparation by Direct Addition

### NF-31, NF-3, NF-33, NF-34, NF-35, NF-36,

1,2-Distearoyl-sn-glycero-3-phosphocholine (DSPC) and 1,2-Dipalmitoyl-sn-glycero-3-phosphocholine (DPPC) were mixed with PFD and PFTA, and the mixture was probe sonicated for about 2 mins. Water or aqueous buffer and non-lipid surfactant (e.g., hexadecyltrimethylammonium bromide (CTAB), Tween 80) if any, were added to above mixture slowly, and the resulted solution was probe sonicated 8 mins at 20 amplitude (e.g., at 45° C.) to afford the titled compositions. The quantity of each component is summarized in Table 1.

### Example 10. Method of Emulsion Preparation by Thin Film Hydration

### NF-44

1,2-Dipalmitoyl-sn-glycero-3-phosphocholine (DPPC), 1,2-Distearoyl-sn-glycero-3-phosphocholine (DSPC) and non-lipid surfactant (e.g., Tween 80) if any, were added to ethanol, and the solution was stirred until became clear. The above solution was transferred into RBF and Rota evaporated, when a thin film was formed. The thin film was then hydrated with water. A mixture of PFD and PFTA were added slowly to above mixture on probe sonication at 20 amplitude 10 mins to afford the titled compositions. The quantity of each component is summarized in Table 1.

### NF-45

1,2-Dipalmitoyl-sn-glycero-3-phosphocholine (DPPC), 1,2-Distearoyl-sn-glycero-3-phosphocholine (DSPC) were added to ethanol and the solution was stirred until became clear. The above solution was transferred into RBF and Rota evaporated to form a thin film. The thin film was then hydrated with required amount of water and surfactant (e.g., Soya Lecithin) if any. Mixture of PFD and PFTA were added slowly to above mixture on Probe sonication at 20 amplitude 10 mins to afford the titled composition. The quantity of each component is summarized in Table 1.

### Example 11. Method of Emulsion Preparation by Direct Addition

### NF-46

1,2-Distearoyl-sn-glycero-3-phosphocholine (DSPC) and 1,2-Dipalmitoyl-sn-glycero-3-phosphocholine (DPPC) was mixed with perfluorocarbon (PFD+PFTA) and the mixture was vortexed. Water was added to above mixture slowly, and the resulted mixture was probe sonicated at 20 amplitude 10 mins to afford NF-46. The quantity of each component is summarized in Table 1.

### NF-47, NF-48

1,2-Distearoyl-sn-glycero-3-phosphocholine (DSPC) and 1,2-Dipalmitoyl-sn-glycero-3-phosphocholine (DPPC) were mixed with water, and the mixture was vortexed. PFD and PFTA were added to above mixture slowly, and the resulted mixture was probe sonicated at 20 amplitude 10 mins. The quantity of each component is summarized in Table 1.

### Example 12. Method of Emulsion Preparation by Thin Film Hydration

### NF-49, NF-50, NF-51, NF-53 and NF-54

Weighed quantity of 1,2-Dipalmitoyl-sn-glycero-3-phosphocholine (DPPC), 1,2-Distearoyl-sn-glycero-3-phosphocholine (DSPC), and HSPC (if any) were added to ethanol, and the mixture was stirred until became clear. The resulting solution was transferred into RBF and Rota evaporated to form a thin film. The thin film was then hydrated with water. A mixture of PFD and PFTA was added slowly to the above mixture on probe sonication at 20 amplitude for 10 mins. The quantity of each component is summarized in Table 1.

### NF-52

1,2-Dipalmitoyl-sn-glycero-3-phosphocholine (DPPC) and 1,2-Distearoyl-sn-glycero-3-phosphocholine (DSPC) were added to ethanol and the mixture was stirred until became clear. The above solution was transferred into RBF, and Rota evaporated to form a thin film. The thin film was then hydrated with water by bath sonication at 65° C. PFD and PFTA were added slowly to the above mixture on probe sonication at 20 amplitude for 10 mins. The quantity of each component is summarized in Table 1.

### Example 13. Method of Emulsion Preparation by Direct Addition

### NF-54

1,2-Distearoyl-sn-glycero-3-phosphocholine (DSPC), 1,2-Dipalmitoyl-sn-glycero-3-phosphocholine (DPPC), and L-a-Phosphatidylcholine, Hydrogenated (soy) (HSPC) were mixed and vortexed. Required quantity of perfluorocarbon (PFD+PFTA) mixture added to above mixture slowly, probe sonicated at 20 amplitude 10 mins. The quantity of each component is summarized in Table 1.

### Example 14. Method of Emulsion Preparation by Direct Addition with Microfluidizer

### NF-55, NF-57

1,2-Distearoyl-sn-glycero-3-phosphocholine (DSPC), HSPC and 1,2-Dipalmitoyl-sn-glycero-3-phosphocholine (DPPC) was mixed with PFD and PFTA and the mixture was vortexed. Water was added to above mixture slowly and vortexed, and the mixture was then microfluidized at 25000 PSI for 2 cycles to afford NF-55. The quantity of each component is summarized in Table 1.

### NF-56

1,2-Distearoyl-sn-glycero-3-phosphocholine (DSPC), HSPC and 1,2-Dipalmitoyl-sn-glycero-3-phosphocholine (DPPC) were mixed with PFD and PFTA and vortexed. Water and non-lipid surfactant (e.g., Soya lecithin) were added to the above mixture slowly and microfluidized at 25000 PSI for 2 cycles and 4 cycles. The quantity of each component is summarized in Table 1.

### NF-58

1,2-Distearoyl-sn-glycero-3-phosphocholine (DSPC), HSPC and 1,2-Dipalmitoyl-sn-glycero-3-phosphocholine (DPPC) was mixed with PFD and PFTA on overhead stirring at 3.5 rpm. Water was added to above mixture slowly and the solution was stirred for 30 mins. The resulted mixture was then microfluidized at 25000 PSI for 2 cycles, 3 cycles and/or 4 cycles. The quantity of each component is summarized in Table 1.

### NF-59

1,2-Distearoyl-sn-glycero-3-phosphocholine (DSPC) and 1,2-Dipalmitoyl-sn-glycero-3-phosphocholine (DPPC) were mixed with PFD+PFTA. The resulted mixture was probe homogenized at 3.5 rpm for 1 min. Water was added to above mixture slowly under probe homogenization at 3.4 rpm for 19 mins. The quantity of each component is summarized in Table 1.

### NF-60 to NF-79

1,2-Distearoyl-sn-glycero-3-phosphocholine (DSPC) and 1,2-Dipalmitoyl-sn-glycero-3-phosphocholine (DPPC) was mixed with PFD and PFTA, and the mixture was homogenized for 1 min. Water or buffer was added to above mixture under homogenization for 1 min at 3.4 rpm. The resulted mixture was then microfluidized at 25000 PSI for 3 cycles, 4 cycles, or 5 cycles to afford the titled compositions. Optionally, the microfluidized mixture was cooled between cycles. The compositions were filled into type 1 glass vials with rubber stoppers and sealed. The vials were sterilized by autoclave method at 121° C. for 20 min at 15 psi. The quantity of each component is summarized in Table 1.

### NF-74

PFD and PFTPA were added into the manufacturing vessel. 1,2-Dipalmitoyl-sn-glycero-3-phosphocholine) (DPPC) and (1,2-Distearoyl-sn-glycero-3-phosphocholine) (DSPC) were added to the perfluorocarbon mixture at 3.5 rpm using probe homogenizer. To the above solution, buffer was added and the mixture was stirred to get a homogenous solution at 3.5 rpm using probe homogenizer. Further, the solution was subjected to micro fluidization at 25000 psi for 3 cycles at RT. The obtained bulk solution was filled into 5.0 mL amber-colored glass vials sealed with a coated rubber stopper. The bulk solution filled in glass vials were subjected to terminal sterilization for 20 min at 121° C.

### NF-83

PFD and PFTPA were added into the stainless-steel container. 1,2-Dipalmitoyl-sn-glycero-3-phosphocholine) (DPPC) and (1,2-Distearoyl-sn-glycero-3-phosphocholine) (DSPC) were added to the perfluorocarbon at 3.5 rpm under probe homogenization for 2 mins To the above solution, the buffer was added and stirred to get a homogenous solution under 3.5 rpm probe homogenization until the whole mixture no longer had phase separation. Further, the solution was subjected to microfluidization at 25000 psi for 3 cycles. The resulting bulk solution was filled into clear glass vials sealed with a coated rubber stopper. The vials were sterilized by autoclave at 121° C. for 20 min at 15 psi.

For NF-83, the ratios between various components are summarized in Table A below:

TABLE A

| Ingredients | Concentration (g/g) |
|---|---|
| Perfluorodecalin (PFD) | 0.355 |
| Perfluorotri-n-propylamine (PFTPA) | 0.145 |
| Distearoylphosphatidylcholine (DSPC) | 0.006 |
| Dipalmitoylphosphatidylcholine (DPPC) | 0.017 |
| Sodium Citrate Dihydrate | 0.012 |
| Citric Acid | 0.001 |
| Water | 0.464 |

TABLE 1

| Composition | NF1 | NF2 | NF3 | NF4 | NF5 | NF6 | NF7 | NF8 | NF9 | NF10 | NF11 |
|---|---|---|---|---|---|---|---|---|---|---|---|
| Manufacturing | Rotary | Homoge- | Homoge- | Homoge- | Probe | Probe | Probe | Probe | Probe | Probe | Probe |
| Method | evaporation | nization | nization | nization | soni- | soni- | soni- | soni- | soni- | soni- | soni- |
| | | | | | cation | cation | cation | cation | cation | cation | cation |
| PDF (gm) | 7 | 7 | 7 | 7 | 3.5 | 3.5 | 3.5 | 3.5 | 3.5 | 3.5 | 3.5 |
| PDF (ml) | 3.65 | 3.65 | 3.65 | 3.65 | 1.8 | 1.8 | 1.8 | 1.8 | 1.8 | 1.8 | 1.8 |
| Perfluoro- | 2.98 | 2.98 | 2.98 | 2.98 | 1.5 | 1.5 | 1.5 | 1.5 | 1.5 | 1.5 | 1.5 |
| tripropylamine (gm) | | | | | | | | | | | |
| PTFA (mL) | 1.64 | 1.64 | 1.64 | 1.64 | 0.0823 | 0.0823 | 0.0823 | 0.0823 | 0.0823 | 0.0823 | 0.0823 |
| CTAB (gm) | 0.005 | 0.005 | 0.005 | 0.005 | 0.0075 | 0.005 | 0.005 | 0.0075 | | | |
| Tween 80 (gm) | | | | | | | | | 0.05 | 0.05 | 0.05 |
| DPPC (gm) | 0.025 | 0.025 | 0.025 | 0.025 | 0.025 | 0.025 | 0.025 | 0.025 | 0.025 | 0.025 | 0.025 |
| DSPC (gm) | 0.025 | 0.025 | 0.025 | 0.025 | 0.025 | 0.025 | 0.025 | 0.025 | 0.025 | 0.025 | 0.025 |
| Water | 2.3 | | 2.3 | | 2.3 | | 2.3 | | 2.3 | | 2.3 |
| pH 6.0 Citrate | | 2.3 | | 2.3 | | 2.3 | | 2.3 | | 2.3 | |
| Buffer (mL) | | | | | | | | | | | |

| Composition | NF12 | NF13 | NF14 | NF15 | NF16 | NF17 | NF18 | NF19 | NF20 | NF21 | NF22 |
|---|---|---|---|---|---|---|---|---|---|---|---|
| Manufacturing | Probe | Probe | Probe | Homoge- | Homoge- | Homoge- | Homoge- | Thin film | Direct | Direct | Thin film |
| Method | soni- | soni- | soni- | nization | nization | nization | nization | hydration | Addition | Addition | hydration |
| | cation | cation | cation | | | | | | | | |
| PDF (gm) | 3.5 | 3.5 | 3.5 | 3.5 | 3.5 | 3.5 | 3.5 | 3.5 | 3.5 | 3.5 | 3.5 |
| PDF (ml) | 1.8 | 1.8 | 1.8 | 1.8 | 1.8 | 1.8 | 1.8 | 1.8 | 1.8 | 1.8 | 1.8 |
| Perfluoro- | 1.5 | 1.5 | 1.5 | 1.5 | 1.5 | 1.5 | 1.5 | 1.5 | 1.5 | 1.5 | 1.5 |
| tripropylamine (gm) | | | | | | | | | | | |
| PTFA (mL) | 0.0823 | 0.0823 | 0.0823 | 0.0823 | 0.0823 | 0.0823 | 0.0823 | 0.0823 | 0.0823 | 0.0823 | 0.0823 |
| Tween 80 (gm) | 0.075 | 0.075 | 0.05 | 0.05 | | 0.05 | 0.05 | 0.05 | | | |
| Soya Lecithin (gm) | | | 0.05 | 0.05 | 0.05 | | | 0.05 | 0.05 | 0.05 | 0.05 |
| DPPC (gm) | 0.025 | 0.025 | 0.025 | 0.025 | 0.025 | 0.025 | 0.025 | 0.025 | 0.025 | 0.025 | 0.025 |
| DSPC (gm) | 0.025 | 0.025 | 0.025 | 0.025 | 0.025 | 0.025 | 0.025 | 0.025 | 0.025 | 0.025 | 0.025 |
| Water | 2.3 | 2.3 | 2.3 | 2.3 | 2.3 | 1.6 | 4.6 | 2.3 | 2.3 | 2.3 | 1.7 |

| | NF23 | NF24 | NF25 | NF26 | NF27 | NF28 | NF29 | NF30 | NF31 | NF32 | NF-33 |
|---|---|---|---|---|---|---|---|---|---|---|---|
| Manufacturing | Thin | Thin | Direct | Thin | Thin | Thin | Thin | Thin | Thin | Direct | Direct |
| Method | film | film | Addition | film | film | film | film | film | film | Addition | Addition |
| | hy- | hy- | | hy- | hy- | hy- | hy- | hy- | hy- | | |
| | dration | dration | | dration | dration | dration | dration | dration | dration | | |
| PDF (gm) | 3.5 | 3.5 | 3.5 | 3.5 | 3.5 | 3.5 | 3.5 | 21 | 21 | 21 | 3.5 |
| PDF (ml) | 1.8 | 1.8 | 1.8 | 1.8 | 1.8 | 1.8 | 1.8 | 10.81 | 10.81 | 10.81 | 1.8 |
| Perfluoro- | 1.5 | 1.5 | 1.5 | 1.5 | 1.5 | 1.5 | 1.5 | 9 | 9 | 9 | 1.50 |
| tripropylamine (gm) | | | | | | | | | | | |
| PTFA (mL) | 0.0823 | 0.0823 | 0.0823 | 0.0823 | 0.0823 | 0.0823 | 0.0823 | 4.94 | 4.94 | 4.94 | 0.823 |
| CTAB (gm) | | | | | | | | | | | 7.5 mg |
| DPPC (gm) | 0.025 | 0.05 | 0.025 | 0.025 | 50 | 0.025 | 0.025 | 300 | 300 | 300 | 25 mg |
| DSPC (gm) | 0.025 | 0.05 | 0.025 | 75 | 100 | 0.025 | 0.025 | 300 | 300 | 600 | 25 mg |
| HSPC | | | 0.025 | | | 50 | 0.025 | 300 | | | |
| Water | 1.15 | 2.3 | 2.3 | 1 | 2.3 | 2.3 | 2.3 | 14.25 | 14.25 | 14.25 | NA |
| pH 6.0 Citrate | | | | | | | | | | | 2.32 |
| Buffer (mL) | | | | | | | | | | | |

| | NF-34 | NF-35 | NF-36 | NF-37 | NF-38 | NF-39 | NF-40 | NF-41 | NF-42 | NF-43 | NF-44 |
|---|---|---|---|---|---|---|---|---|---|---|---|
| Manufacturing | Direct | Direct | Direct | Direct | Direct | Direct | Direct | Direct | Direct | Direct | Direct |
| Method | Addi- | Addi- | Addi- | Addi- | Addi- | Addi- | Addi- | Addi- | Addi- | Addi- | Addi- |
| | tion | tion | tion | tion | tion | tion | tion | tion | tion | tion | tion |
| PDF (gm) | 3.5 | 3.5 | 3.5 | 3.5 | 3.5 | 3.5 | 3.5 | 3.5 | 3.5 | 3.5 | 3.5 |
| PDF (ml) | 1.8 | 1.8 | 1.8 | 1.8 | 1.8 | 1.8 | 1.8 | 1.8 | 1.8 | 1.8 | 1.8 |
| Perfluoro- | 1.50 | 1.50 | 1.50 | 1.50 | 1.50 | 1.50 | 1.50 | 1.50 | 1.50 | 1.50 | 1.50 |
| tripropylamine (gm) | | | | | | | | | | | |
| PTFA (mL) | 0.823 | 0.823 | 0.823 | 0.823 | 0.823 | 0.823 | 0.823 | 0.823 | 0.823 | 0.823 | 0.823 |
| Tween 80 | 50 mg | 50 mg | 50 mg | 75 mg | 30 mg | 50 mg | 50 mg | NA | 50 mg | 50 mg | 50 mg |
| Soya Lecithin (gm) | | | | | | | | | | | |
| DPPC | 25 mg | 25 mg | 25 mg | 25 mg | 25 mg | 25 mg | 25 mg | 25 mg | 25 mg | 25 mg | 25 mg |
| DSPC | 25 mg | 25 mg | 25 mg | 25 mg | 25 mg | 25 mg | 25 mg | 25 mg | 25 mg | 25 mg | 25 mg |
| Water | 2.32 mL | NA | 2.32 mL | 2.32 mL | 2.32 mL | 2.32 mL | 1.2 mL | 2.32 mL | 1.6 mL | 4.6 mL | 1.2 mL |
| pH 6.0 Citrate | | 2.32 mL | | | | | | | | | |
| Buffer (mL) | | | | | | | | | | | |

| | NF-45 | NF-46 | NF-47 | NF-48 | NF-49 | NF-50 | NF-51 | NF52 | NF53 | NF54 | NF55 |
|---|---|---|---|---|---|---|---|---|---|---|---|
| Manufacturing | Thin | Thin | Direct | Direct | Direct | Thin | Thin | Thin | Thin | Direct | MF - |
| Method | film | film | Addi- | Addi- | Addi- | film | film | film | film | Addi- | Direct |
| | hy- | hy- | tion | tion | tion | hy- | hy- | hy- | hy- | tion | Addition |
| | dration | dration | | | | dration | dration | dration | dration | | |
| PDF (gm) | 3.5 | 3.5 | 3.5 | 3.5 | 3.5 | 3.5 | 3.5 | 3.5 | 3.5 | 3.5 | 21 |
| PDF (ml) | 1.8 | 1.8 | 1.8 | 1.8 | 1.8 | 1.8 | 1.8 | 1.8 | 1.8 | 1.8 | 10.81 |
| Perfluoro- | 1.50 | 1.50 | 1.50 | 1.50 | 1.50 | 1.50 | 1.50 | 1.5 | 1.5 | 1.5 | 9 |
| tripropylamine (gm) | | | | | | | | | | | |
| PTFA (mL) | 0.823 | 0.823 | 0.823 | 0.823 | 0.823 | 0.823 | 0.823 | | | | 4.94 |
| DPPC | 25 mg | 25 mg | 25 mg | 25 mg | 50 mg | 25 mg | 75 mg | 100 mg | 25 mg | 0.025 | 300 mg |
| | | | | | | | | | | | (50 mg/5 mL) |
| DSPC | 25 mg | 25 mg | 25 mg | 25 mg | 50 mg | 25 mg | 25 mg | 50 mg | 25 mg | 0.025 | 300 mg |
| | | | | | | | | | | | (50 mg/5 mL) |
| HSPC | | | | | | | | | 50 mg | 25 | 300 mg |
| | | | | | | | | | | | (50 mg/5 mL) |
| Water | 2.32 mL | 2.32 mL | 1.7 mL | 1.17 mL | 2.32 mL | 2.32 mL | 1 mL | 2.32 mL | 2.3 | 2.3 | 14.25 |

| | | | | | NF61 | NF61 | NF62 | NF62 | NF63 | NF64 |
|---|---|---|---|---|---|---|---|---|---|---|
| | NF56 | NF57 | NF58 | NF60 | (5 gm) | (100 gm) | (5 gm) | (60 gm) | (60 g) | (67.27 g) |
| Manufacturing | MF - | MF - | MF - | MF - | MF - | MF - | MF - | MF - | MF - | MF - |
| Method | Direct | Direct | Direct | Direct | Direct | Direct | Direct | Direct | Direct | Direct |
| | Addition | Addition | Addition | Addition | Addition | Addition | Addition | Addition | Addition | Addition |
| PDF (gm) | 21 | 21 | 21 | 21 | 2.50 | 50.00 | 2.50 | 30.00 | 30 | 30 |
| PDF (ml) | 10.81 | 10.81 | 10.81 | 10.81 | 1.30 | 26.04 | 1.30 | 15.62 | | |
| Perfluoro- | 9 | 9 | 9 | 9 | 1.00 | 20.00 | 1.00 | 12.00 | 12 | 12 |
| tripropylamine (gm) | | | | | | | | | | |
| PTFA (mL) | 4.94 | 4.94 | 4.94 | 4.94 | 0.54 | 10.98 | 0.54 | 6.59 | | |
| Soya Lecithin (gm) | 300 mg | | | | | | | | | |
| | (50 mg/ | | | | | | | | | |
| | 5 mL) | | | | | | | | | |
| DPPC (gm) | 300 mg | 300 mg | 300 mg | 300 mg | 0.035 | 0.70 | 0.035 | 0.42 | 0.42 | 0.42 |
| | (50 mg/ | (50 mg/ | (50 mg/ | (50 mg/ | | | | | | |
| | 5 mL) | 5 mL) | 5 mL) | 5 mL) | | | | | | |
| DSPC (gm) | 300 mg | 600 mg | 900 mg | 900 mg | 0.10 | 2.00 | 0.10 | 1.2 | 1.2 | 1.2 |
| | (50 mg/ | (100 mg/ | (150 mg/ | (150 mg/ | | | | | | |
| | 5 mL) | 5 mL) | 5 mL) | 5 mL) | | | | | | |
| Water | 14.25 | 14.25 | 14.25 | 14.25 | | | 1.365 | 16.38 | | |
| Batch size (gm) | | | | | 5.00 | 100.0 | 5.00 | 60.00 | | |
| Batch size (mL) | | | | | 3.34 | 67.02 | 3.34 | 40.21 | | |
| pH 6.0 Citrate | | | | | | | | | 16.38 | 23.65 |
| Buffer (mL) | | | | | | | | | | |
| pH 6.8 Phosphate | | | | | 1.365 | 27.30 | | | | |
| buffer (mL) | | | | | | | | | | |

| | NF65 | NF65 | NF66 | NF66 | NF67 | NF67 | NF68 | NF68 | NF69 | NF69 | NF70 |
|---|---|---|---|---|---|---|---|---|---|---|---|
| | (1 gm) | (~135 gm) | (1 gm) | (~260 gm) | (1 gm) | (110 gm) | (1 gm) | (~260 gm) | (1 gm) | (50 gm) | (1 gm) |
| Manufacturing | MF - | MF - | MF - | MF - | MF - | MF - | MF - | MF - | MF - | MF - | MF - |
| Method | Direct | Direct | Direct | Direct | Direct | Direct | Direct | Direct | Direct | Direct | Direct |
| | Addi- | Addi- | Addi- | Addi- | Addi- | Addi- | Addi- | Addi- | Addi- | Addi- | Addi- |
| | tion | tion | tion | tion | tion | tion | tion | tion | tion | tion | tion |
| PDF (gm) | 0.445 | 60.000 | 0.445 | 115.000 | 0.445 | 50.000 | 0.445 | 115.7 | 0.381 | 19.050 | 0.445 |
| PDF (ml) | 0.232 | 31.250 | 0.232 | 59.895 | 0.232 | 26.041 | 0.232 | 60.26 | 0.198 | 9.922 | 0.232 |
| Perfluoro- | 0.178 | 24.000 | 0.178 | 46.000 | 0.178 | 20.000 | 0.178 | 46.280 | 0.151 | 7.550 | 0.178 |
| tripropylamine (gm) | | | | | | | | | | | |
| PTFA (mL) | 0.098 | 13.187 | 0.098 | 25.274 | 0.098 | 10.989 | 0.098 | 25.428 | 0.083 | 4.148 | 0.098 |
| DPPC (gm) | 0.006 | 0.840 | 0.006 | 1.610 | 0.006 | 0.700 | 0.006 | 1.520 | 0.006 | 0.300 | 0.006 |
| DSPC (gm) | 0.017 | 2.400 | 0.017 | 4.600 | 0.017 | 2.000 | 0.017 | 4.420 | 0.017 | 0.850 | 0.017 |
| Calcium Chloride | | | | | | | | | | | 0.002 |
| Batch size (gm) | 1.000 | 134.540 | 1.000 | 257.860 | 1.0 | 112.11 | 1.000 | 260.000 | 1.000 | 50.000 | 1.000 |
| Batch size (mL) | 0.707 | 94.977 | 0.707 | 182.029 | 0.707 | 79.140 | 0.707 | 183.668 | 0.749 | 37.470 | 0.707 |
| pH 6.0 Citrate | 0.354 | 47.300 | 0.354 | 90.650 | 0.354 | 39.410 | 0.354 | 92.040 | 0.445 | 22.250 | 0.352 |
| Buffer (mL) | | | | | | | | | | | |

| | NF70 | NF71 | NF71 | NF72 | NF72 | NF73 | NF73 | NF74 | NF74 | NF75 | NF75 |
|---|---|---|---|---|---|---|---|---|---|---|---|
| | (50 gm) | (1 gm) | (50 gm) | (1 gm) | (50 gm) | (1 gm) | (150 gm) | (1 gm) | (50 gm) | (1 gm) | (50 gm) |
| Manufacturing | MF - | MF - | MF - | MF - | MF - | MF - | MF - | MF - | MF - | MF - | MF - |
| Method | Direct | Direct | Direct | Direct | Direct | Direct | Direct | Direct | Direct | Direct | Direct |
| | Addi- | Addi- | Addi- | Addi- | Addi- | Addi- | Addi- | Addi- | Addi- | Addi- | Addi- |
| | tion | tion | tion | tion | tion | tion | tion | tion | tion | tion | tion |
| PDF (gm) | 22.25 | 0.445 | 22.250 | 0.355 | 17.750 | 0.445 | 66.750 | 0.355 | 53.250 | 0.355 | 17.750 |
| PDF (ml) | 11.589 | 0.232 | 11.588 | 0.185 | 9.244 | 0.232 | 34.765 | 0.185 | 27.734 | 0.185 | 9.244 |
| Perfluoro- | 8.900 | 0.178 | 8.900 | 0.145 | 7.250 | 0.178 | 26.700 | 0.145 | 21.750 | 0.145 | 7.250 |
| tripropylamine (gm) | | | | | | | | | | | |
| PTFA (mL) | 4.890 | 0.098 | 4.890 | 0.080 | 3.984 | 0.098 | 14.670 | 0.080 | 11.951 | 0.080 | 3.984 |
| DPPC (gm) | 0.300 | 0.006 | 0.300 | 0.006 | 0.300 | 0.006 | 0.900 | 0.006 | 0.900 | 0.006 | 0.300 |
| DSPC (gm) | 0.850 | 0.017 | 0.850 | 0.017 | 0.850 | 0.017 | 2.550 | 0.017 | 2.550 | 0.017 | 0.850 |
| Calcium Chloride | 0.100 | | | | | | | | | | |
| Batch size (gm) | 50.000 | 1.000 | 50.000 | 1.000 | 50.000 | 1.000 | 150.000 | 1.000 | 150.000 | 1.000 | 50.000 |
| Batch size (mL) | 35.329 | 0.707 | 35.328 | 0.765 | 38.228 | 0.707 | 105.985 | 0.765 | 114.685 | 0.765 | 38.228 |
| pH 6.0 Citrate | 17.600 | 0.354 | 17.700 | 0.477 | 23.850 | 0.354 | 53.100 | 0.477 | 71.550 | 0.477 | 23.850 |
| Buffer (mL) | | | | | | | | | | | |

| | NF76 | NF76 | NF77 | NF77 | | | NF76 | NF76 | NF77 | NF77 | | |
|---|---|---|---|---|---|---|---|---|---|---|---|---|
| | (1 gm) | (100 gm) | (1 gm) | (100 gm) | NF78 | NF79 | (1 gm) | (100 gm) | (1 gm) | (100 gm) | NF78 | NF79 |
| Manufacturing | MF - | MF - | MF - | MF - | MF - | MF - | MF - | MF - | MF - | MF - | MF - | MF - |
| Method | Direct | Direct | Direct | Direct | Direct | Direct | Direct | Direct | Direct | Direct | Direct | Direct |
| | Addi- | Addi- | Addi- | Addi- | Addi- | Addi- | Addi- | Addi- | Addi- | Addi- | Addi- | Addi- |
| | tion | tion | tion | tion | tion | tion | tion | tion | tion | tion | tion | tion |
| PDF (gm) | 0.355 | 35.500 | 0.355 | 35.500 | 53.25 | 131.35 | 0.355 | 35.500 | 0.355 | 35.500 | 53.25 | 131.35 |
| PDF (ml) | 0.370 | 18.489 | 0.370 | 18.489 | | | 0.370 | 18.489 | 0.370 | 18.489 | | |
| Perfluoro- | 0.145 | 14.500 | 0.145 | 14.500 | 21.75 | 53.65 | 0.145 | 14.500 | 0.145 | 14.500 | 21.75 | 53.65 |
| tripropylamine (gm) | | | | | | | | | | | | |
| PTFA (mL) | 0.159 | 7.967 | 0.159 | 7.967 | | | 0.159 | 7.967 | 0.159 | 7.967 | | |
| DPPC (gm) | 0.006 | 0.600 | 0.006 | 0.600 | 0.9 | 2.22 | 0.006 | 0.600 | 0.006 | 0.600 | 0.9 | 2.22 |
| DSPC (gm) | 0.017 | 1.700 | 0.017 | 1.700 | 2.55 | 6.29 | 0.017 | 1.700 | 0.017 | 1.700 | 2.55 | 6.29 |
| Batch size (gm) | 1.000 | 100.000 | 1.000 | 100.000 | | | 1.000 | 100.000 | 1.000 | 100.000 | | |
| Batch size (mL) | 1.029 | 76.456 | 1.029 | 76.456 | | | 1.029 | 76.456 | 1.029 | 76.456 | | |
| pH 6.0 Citrate | 0.477 | 47.700 | 0.477 | 47.700 | 71.55 | 176.49 | 0.477 | 47.700 | 0.477 | 47.700 | 71.55 | 176.49 |
| Buffer (mL) | | | | | | | | | | | | |

### Example 15. Characterization of the Prepared Nanoparticle Compositions

The nanoparticle compositions prepared in Example 1 to Example 14 were further tested for phase stability, pH, viscosity, dissolved oxygen, particle size, PDI, and zeta potential. Observations on the phase stability for each nanoparticle composition are summarized in Table 2.

### Particle Size and Zeta Potential Measurement

The prepared nanoparticle compositions were investigated for particle size, polydispersity index, and surface potential by dynamic light scattering technique. The particle size was determined using cell (dia. 10 mm) at 25° C. The prepared nanoparticle compositions were diluted (1:5) with citrate buffer pH 6.0 prior to the size measurement studies in order to ensure the free Brownian motion of the droplets.

### Viscosity Measurement

The viscosity of the prepared nanoparticle compositions was determined using Brookfield cone and plate rheometer (Make: Brookfield Engineering Laboratory, Model: DV2T) at a constant speed of 80 rpm using spindle #CPE40 at 25±0.5° C. The software used for the calculations was done by Rheocalc V2.6. All the experiments were performed in triplicates.

### pH Measurement

The pH value of the prepared nanoparticle compositions was determined using a Mettler Toledo pH meter. Before measurement, the equipment was calibrated with buffer solutions of pH 4.00±0.05, pH 7.00±0, and pH 10.00±0.05. The pH measurements were carried out in triplicate at room temperature.

### Osmolality

Using an Osmometer (Make: Vapro, Model: 5600) by freezing point measurement technique, the osmolality was determined. The osmometer supplier's standardizing solution was used to calibrate the instrument. 100 μL of the evaluated nanoparticle samples were put in dedicated microtubes before being inserted into the measurement head.

### Cryogenic Electron Microscopy, Cryo-TEM

Cryo-TEM was used to provide accurate depictions of nanoparticle shape, size, and integrity in their diluted condition by freezing the sample. Nanoparticle was diluted with lung fluid in 1:5 ratios to minimize droplet overlays and therefore enable following particle size measurement. A charge of 25 mA per 50 seconds was applied to gold grids with carbon film type Lacey, 300 mesh. The grids were then loaded into the Vitrobot Mark IV sample vitrification robot. Samples diluted with lung fluid at a ratio of 1:5 and 1:10 were put to the grid and the excess was removed using a blot with a duration of 8.5 seconds and a force of −15. The grids were immediately frozen in liquid ethane and kept in liquid nitrogen until the microscope examination. Images were captured using a 200 kV-operated Talos Arctica TEM model. The microscope was equipped with a camera for acquisition of digital images.

### Stability of the Nanoparticle Composition

By recommendations of the International Conference on Harmonisation (ICH), accelerated stability experiments were performed on the prepared compositions. The perfluorocarbon-loaded compositions were stored under long term and accelerated conditions, 25° C./60% RH and 40° C./75% RH for 24 weeks and observed at weeks 0, 4, 8, 12 and 24. The physicochemical stability test of the nanoparticle compositions was performed by observing the changes in appearance, pH, viscosity, osmolality, phase separation, particle size, polydispersity index, zeta potential, assay and purity.

The conditions used for stability tests were:
Long term (25° C.±2° C./60% RH±5% RH) for 3 months and 6 months
Accelerated (40° C.±2° C./75% RH±5% RH) for 1 month, 2 months, 3 months and 6 months.

### Analysis of Perfluorocarbons in Nanoparticle Compositions

The analyses of perfluorocarbons in nanoparticle compositions were carried out using gas chromatography instrument stands with the following specifications, under a Gas Chromatographs instrument (Make: Shimadzu, Model: Nexis 2030) coupled with a Flame Ionization Detector (FID).

The results are summarized in Table 2.

TABLE 2

| Composition | NF1 | NF2 | NF3 | NF4 | NF5 | NF6 | NF7 | NF8 | NF9 | NF10 |
|---|---|---|---|---|---|---|---|---|---|---|
| Observation | ++++ | + | +++ | + | +++ | + | ++++ | + | + | + |
| Composition | NF11 | NF12 | NF13 | NF14 | NF15 | NF16 | NF17 | NF18 | NF19 | NF20 |
| Observation | + | + | + | + | + | +++ | + | + | + | ++ |
| Composition | NF21 | NF22 | NF23 | NF24 | NF25 | NF26 | NF27 | NF28 | NF29 | NF30 |
| Observation | ++ | ++ | + | + | + | + | + | + | + | +++ |

| | | | | | | | | | | NF61 |
|---|---|---|---|---|---|---|---|---|---|---|
| Composition | NF31 | NF32 | NF52 | NF53 | NF54 | NF55 | NF56 | NF57 | NF60 | (100 gm) |
| Observation | +++ | +++ | + | + | + | +++ | ++++ | +++ | +++ | ++++ |

| | NF63 | NF64 | NF65 | NF67 | NF68 | NF69 | NF70 | NF71 | NF72 | NF73 |
|---|---|---|---|---|---|---|---|---|---|---|
| Composition | (60 g) | (67.27 g) | (~135 gm) | (110 gm) | (~260 gm) | (50 gm) | (50 gm) | (50 gm) | (50 gm) | (150 gm) |
| Observation | ++++ | ++++ | ++++ | ++++ | ++++ | ++++ | ++++ | ++++ | ++++ | ++++ |

| | NF74 | NF75 | NF76 | NF77 |
|---|---|---|---|---|
| Composition | (50 gm) | (50 gm) | (100 gm) | (100 gm) |
| Observation | ++++ | ++++ | ++++ | ++++ |

+ Phase separation on Day 1;
++ Phase separation between Day 1 and Day 4;
+++ No phase separation up to Day 4;
++++ No phase separation up to Day 10

As shown in Table 2, the nanoparticle compositions comprising centrimonium bromide (CTAB), such as NF1 to NF8, the nanoparticle compositions comprising Tween 80, such as NF9 to NF19, the nanoparticle compositions comprising Soya Lecithin, such as NF14-NF16, NF19-NF 23, NF31, and NF56, and the nanoparticle compositions comprising soya phosphatidyl choline (HSPC), such as NF28-NF30, NF54, and NF55, did not show significantly improved physical stability. Some phase separation was observed for these nanoparticle compositions. Surprisingly, the nanoparticle compositions that comprises DSPC and DPPC while does not comprise CTAB, Tween 80, Soya Lecithin, or HSPC, tended to have better stability and much longer number of days before phase separation. It was unexpected that only DSPC/DPPC can form a physically stable nanoparticle composition with perfluorocarbon without additional surfactants or lipids.

By comparing NF61, which comprises phosphate buffer, with NF65 to NF79, which all comprise citrate buffer, it was surprisingly found that citrate buffer, which has much lower toxicity than phosphate buffer, tended to afford more stable nanoparticle compositions. The typical citrate cellular concentration is reasonably high, so animals typically have a high tolerance for citrate buffer.

Other characterization results, including pH, viscosity, dissolved oxygen, particle size, PDI, and zeta potential, for representative nanoparticle compositions are summarized in Table 3.

TABLE 3

| | NF1 | NF2 | NF7 | | NF11 | NF30 | NF31 | NF32 | NF55 | NF56 |
|---|---|---|---|---|---|---|---|---|---|---|
| pH | | 5.86 | 4.72 | | 5.96 | 7.63 | 6.58 | 3.78 | 7.63 | 6.58 |
| Osmolarity | | | | | | 23 | 36 | 13 | 23 | 36 |
| Viscosity | 1767 cP | | 1144 | cP | | 40 | 20 | 13 | 40 | 20 |
| Dissolved Oxygen | | | 8.7 | ppm | | | | | | |

| | | | | | |
|---|---|---|---|---|---|
| Particle size (nm) | 3256 | 2108 | 1219 | 3256 | 2108 |
| PDI | 1 | 1 | 0.678 | 1.0 | 1.0 |
| Zeta potential (mV) | 0.598 | 0.0233 | 0.677 | 0.598 | 0.0233 |

| | | | NF61 | NF62 | NF63 | NF64 | NF65 | NF66 | NF67 |
|---|---|---|---|---|---|---|---|---|---|
| Composition | NF57 | NF60 | (100 gm) | (60 gm) | (60 g) | (67.27 g) | (~135 gm) | (~260 gm) | (110 gm) |
| pH | 3.78 | 6.14 | 6.73 | 6.29 | 6.29 | 6.05 | 5.94 | 5.97 | 5.92 |
| Osmolarity | 13 | | 365 | 22 | 22 | 160 | 237 | 238 | 261 |
| Viscosity | 13 | | 522.5 | 304.5 | 304.5 | 17.0 | 16.0 | 17.0 | 17.8 |
| Dissolved Oxygen | | | NA | NA | NA | NA | 8.638 | 8.299 | 7.643 |
| Particle size (nm) | 1219 | 2096 | | | 1291 | 1318 | | | 943 |
| Particle size (nm) + 5x Citrate | | | | | 309 | 296 | 361 | 320 | 341 |
| PDI | 0.678 | 0.577 | | | | | | | |
| Zeta potential (mV) | 0.677 | 0.123 | | | | | | | |

| | NF68 | NF69 | NF70 | NF71 | NF72 | NF73 | NF74 | | |
|---|---|---|---|---|---|---|---|---|---|
| | (~260 gm) | (50 gm) | (50 gm) | (50 gm) | (50 gm) | (150 gm) | (50 gm) | NF78 | NF79 |
| pH | 5.94 | 5.90 | 5.49 | 6.07 | 6.07 | 5.99 | 6.03 | 6.05 | 6.29 |
| Osmolarity | 297 | 226 | 332 | 247 | 244 | 314 | 264 | 345 | 285 |
| Viscosity | 26.32 | 6.9 | 21.7 | 14.7 | 7.0 | 23.4 | 9.77 | 9.5 | 3.0 |
| Dissolved Oxygen | 8.46 | 8.692 | 8.794 | 9.789 | 9.713 | 8.947 | 8.952 | 9.085 | 9.179 |
| Particle size (nm) | 1898 | 738 | | | | | | | |
| Particle size (nm) + 5x Citrate | 323 | 295 | 309 | 341 | 269 | 322 | 280 | 257 | 273 |

As shown in Table 3, surprisingly, the particle size of the nanoparticles in the nanoparticle compositions could be decreased by increasing the number of passes in the microfluidization process. Further diluting the prepared nanoparticle compositions could shrink the particle size further down to about 300 nm to about 400 nm. Also unexpectedly, increasing the amount of water or aqueous buffer in the nanoparticle composition could decrease the viscosity of the nanoparticle composition. Such a lower viscosity and smaller particle size are generally preferred for inhalable formulations and suitable for nebulization process. As shown in Table 3, the stable nanoparticle compositions had a pH range of about 5 to about 7.5. These compositions typically carried dissolved oxygen at about 7 to about 10 ppm, measured only in aqueous layer, and exhibited a viscosity of about 3 cp to about 500 cp.

FIG. 2 shows CryoEM images for representative nanoparticles in the nanoparticle. The images were captured from different grids with 1:5 dilution with lung fluid. As shown in FIG. 2, the nanoparticles have less than 0.5 μm particle size and exhibit multi-lamellar structure.

In summary, two distinct methods were used to prepare the nanoparticle compositions. These entailed employing microfluidic technology, probe sonicator, probe homogenizer, thin film hydration technique, and several emulsifier types and concentrations. Changes that affect emulsion stability, such as particle size and dispersion, were the key features of a manufactured nanoparticle composition. Various techniques for preparing a stable nanoparticle composition system with surfactants/no surfactants were investigated independently in this study. However, the use of non-lipid surfactants has limitations that might contaminate the product and adversely affect its biocompatibility. To overcome this issue, surfactant-free nano-emulsification methods have been developed. Nanoparticle compositions can be formed in the absence of surfactants via a microfluidic technique based on high shear energy.

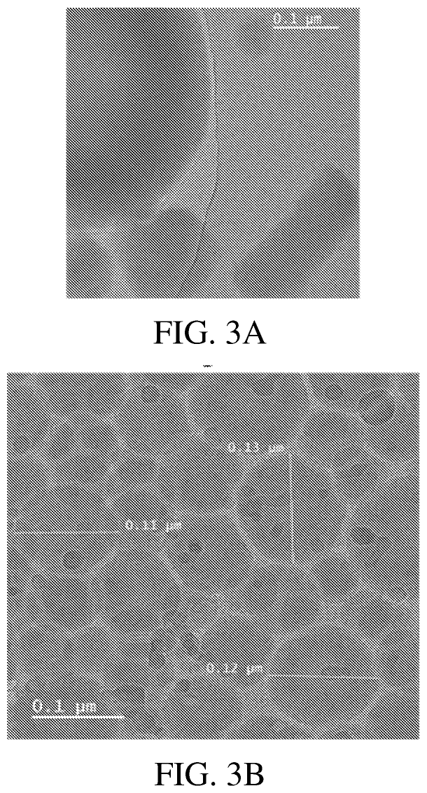

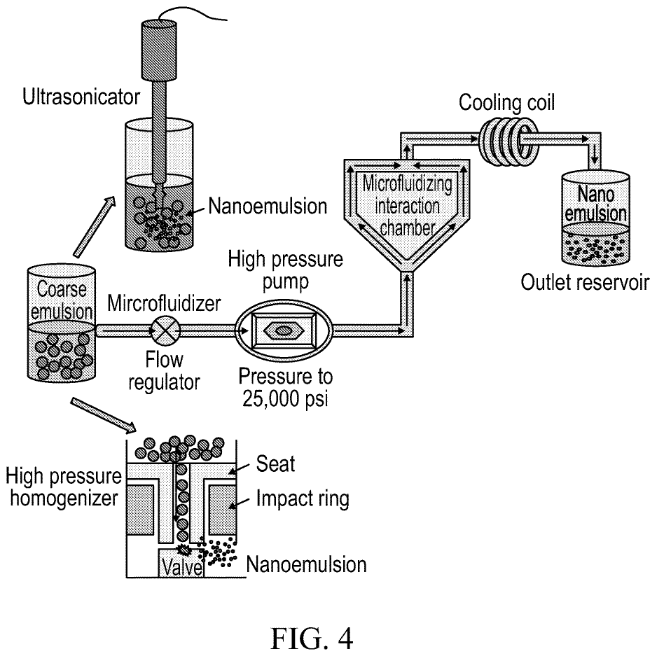

A morphology examination of the nanoparticle compositions developed was performed using Cryo TEM; a technique that captures simultaneous structures and microstructure transitions with high resolution images. The Cryo TEM images of NF-66 showed the spherical appearance of droplets with uniform size (FIG. 3B). These nanoparticle compositions were found to be stable for up to 6 months at 25° C./60% RH storage.

The nanoparticle composition NF-62 was highly viscous in presence of water as continuous phase. As evident by Cryo-TEM image FIG. 3A, effect of Ostwald ripening in this preparation leads to shrinkage or disappearance of smaller sized droplets, while bigger sized droplets grew further leading to phase separation on Day 28. NF-66 (FIG. 3B), which was prepared with more PFC and using Citrate buffer showed smaller sized (compared with (FIG. 3A)). DLS data revealed a slightly increased hydrodynamic size 2.2 μm and PDI of 0.6 for NF-66. Upon dilution with citrate buffer at 1:5 ratio, mean particle size was reduced to 320 nm and PDI 0.170.

### Example 16. Efficacy of Exemplary Composition in Treating Acute Respiratory Distress (ARDS)

Inducing ARDS with Lipopolysaccharides (LPS)

Mice were administered with LPS (BMS-345541) intratracheally at a dose of 1 mg/kg body weight under sedation. Briefly, the mouse was anesthetized using isoflurane and placed in the supine position with its upper incisor teeth secured using a wire harness. The mice were then intubated intratracheally using a gel loading tip (plastic/polypropylene). A 25 μL sterile saline solution was introduced in the tip to check the proper placement of the loading tip into the trachea. The slow up and down movement of the placed solution confirmed the proper placement of the tip into the trachea. The saline solution was carefully removed using a pipette.

LPS 1 mg/mL stock solution was diluted with sterile saline and administered by intratracheal instillation based on the weight of the mouse. A 25 μm mouse would receive 25 μL of LPS mixed with 75 μL of sterile saline solution premixed in a tube. This solution was then carefully introduced into the tip inserted into the trachea. It was then gently pushed inside the trachea using a positive pressure using a 200 μL pipette. All the mice received a total volume of 100 μL of LPS and saline mix, keeping the total volume administered the same among the animals. 12 mice were intratracheally instilled with LPS and allowed to recover from anaesthesia and were monitored for 24 hrs for signs of distress. One of the mice died after LPS instillation and the other 11 mice were separated in to 2 groups. The animals were grouped as follows:
i) 6 mice with LPS instillation (i.e., diseased) received nanoparticle composition NF-83-100 ML
ii) 5 mice with LPS instillation (i.e., diseased) received sterile saline solution-100 μL

The mice received the treatment for three sessions with a treatment interval of 48 hours. The mice were euthanized 48 hours after the last treatment and the lung tissue from both the sides were harvested. The lung tissue was equally distributed for immunohistochemical analysis and western blotting experiments. For immunohistochemical analysis the tissues were fixed using 4% paraformaldehyde and cryoprotected using 30% sucrose. For western blot analysis the lung tissue was frozen on dry ice and stored at −80° C. freezer.

### Results

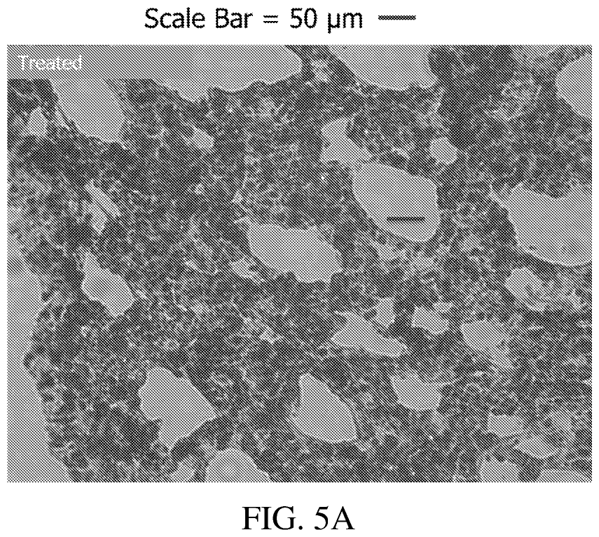

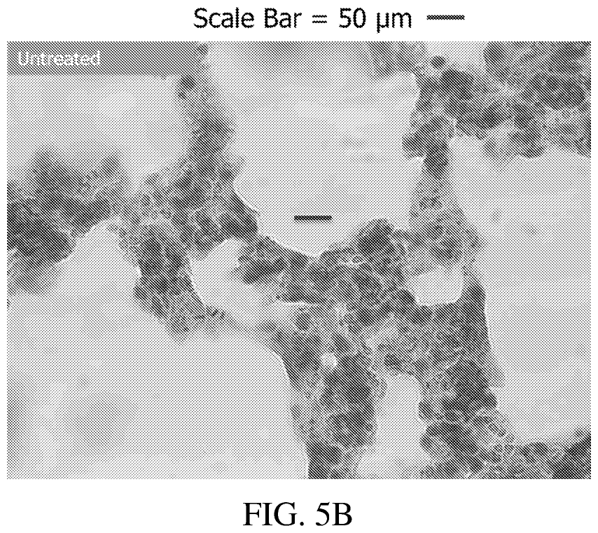

The results are shown in FIG. 5A (treated with NF-83 nanoparticle composition) and FIG. 5B (treated with sterile saline solution). In FIG. 5A and FIG. 5B, the light areas were cross section of alveoli. As shown in FIG. 5A, mice treated with the exemplary nanoparticle composition provided herein exhibited more intact alveoli and less damage to epithelial cells, while as shown in FIG. 5B, the untreated mice exhibited disorganized and/or collapsed alveolar and epithelium damage. These results demonstrated the effect of the exemplary nanoparticle composition in delaying or reversing lung injury and/or destruction of lung tissue, and potentially in treating ARDS.

Treating Mice with Bacterial Infection Induced ARDS

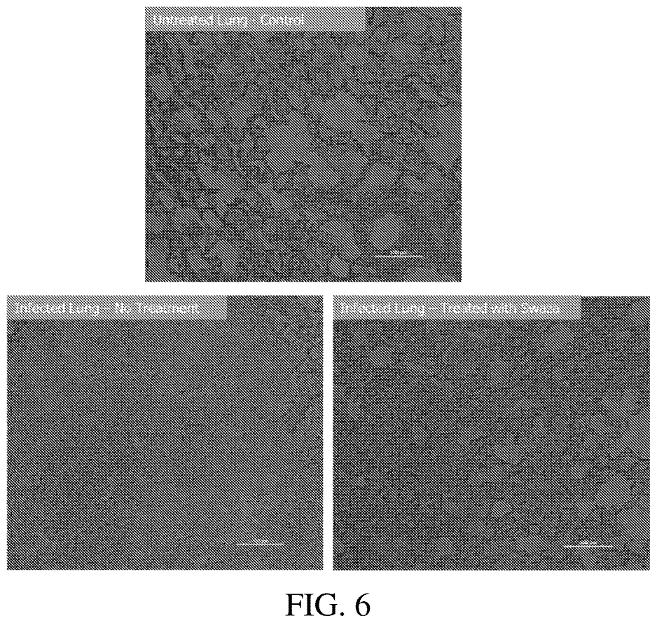

Further to earlier promising results, the treatment protocol was repeated in a new set of mice with a bacterial infection-caused ARDS. The treatment group received the exemplary nanoparticle composition NF-83 on day 3 and day 5 as in the LPS group. According to lung histology results, the exemplary nanoparticle composition NF-83 treatment significantly reduced the degradation of alveolar structures in the infected and treated mice as compared to untreated infected mice. The results are shown in FIG. 6.

### Biomarker Expression

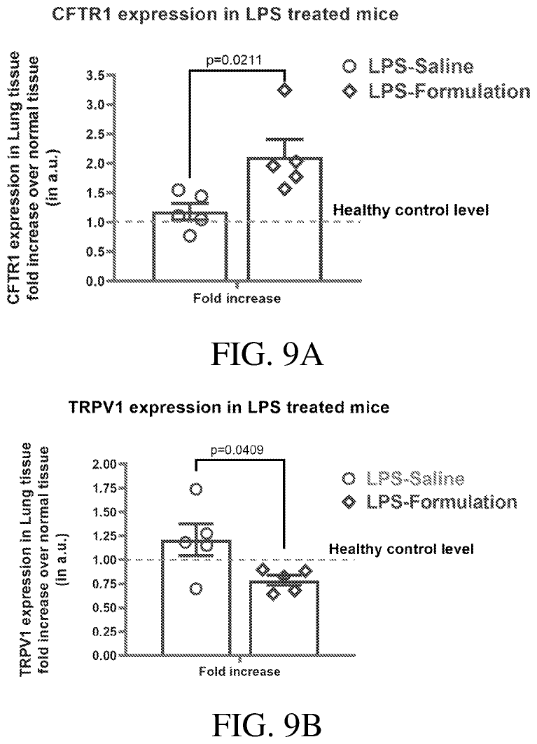

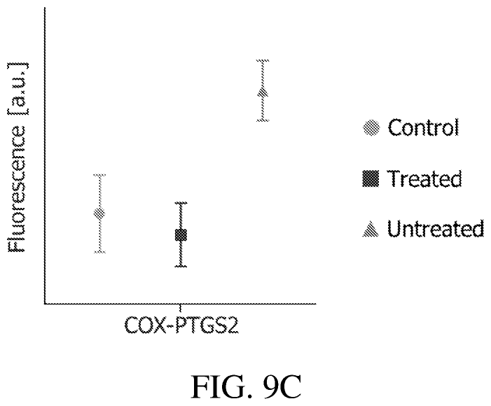

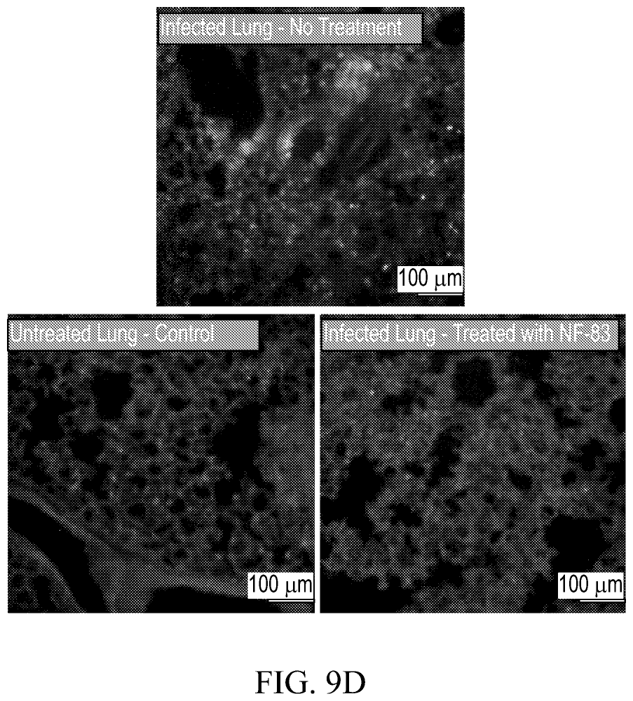

As shown in FIG. 9A, the exemplary nanoparticle composition NF-83 also increased CFTR1 expression in LPS Treated Mice. While CFTR is primarily associated with cystic fibrosis (CF), a genetic disorder that affects the lungs and other organs, recent research has suggested that CFTR may also play a role in the pathophysiology of ARDS. Studies have shown that increased CFTR expression in the lung can have a protective effect in ARDS. These results demonstrated NF-83's capacity to target CFTR signaling pathways and its potential as a therapeutic strategy for ARDS.

Further, as shown in FIG. 9B, the exemplary nanoparticle composition NF-83 decreased TRPV1 expression in LPS Treated Mice. TRPV1 (Transient Receptor Potential Vanilloid 1) is a member of the transient receptor potential (TRP) family of ion channels that is expressed in various tissues including the lung. Studies suggest that suppressing the suppression of TRPV1 reduces lung inflammation and injury. TRPV1 may also play a role in the pathophysiology of Acute Respiratory Distress Syndrome (ARDS), a life-threatening condition characterized by severe lung inflammation and impaired oxygenation. Studies have found that TRPV1 is expressed in lung tissue and that its activation can contribute to lung inflammation and injury. TRPV1 has also been implicated in the development of pulmonary edema, a hallmark of ARDS in which fluid accumulates in the lungs.

In animal models of ARDS, blocking TRPV1 has been shown to reduce lung inflammation and improve oxygenation, suggesting that TRPV1 antagonists may have potential as a therapeutic target for ARDS. With the capacity to decrease TRPV1 in animals with induced ARDS, NF-83 was demonstrated to be a promising treatment agent for ARDS.

In addition, it was also observed that treatment with NF-83 suppressed signals of inflammation associated with hypoxia. Elevated COX-PTGS2 is a marker of inflammation associated with hypoxia. Study of control, infected, and treated mouse lung tissue showed treatment with NF-83 resulted in statistically significant suppression of COX-PTGS2 expression, indicating its oxygenation effect (FIG. 9C, FIG. 9D).

### Example 17. Dynamics of Nanoparticle Driven by Tidal Breathing Pattern of the Lung

To assess NF-83's shuttling functionality for oxygen delivery and carbon dioxide removal, a StimuLung lung-on-a-chip model was used in which oxygen flow and CO₂ uptake were measured using fluorescent dyes, which could mimic exposure of the nanoparticles in the nanoparticle composition to air and exposure to deoxygenated blood. The chip had a transparent and flexible substrate containing microchannels mimicking air sacs and capillaries of the lungs. These microchannels were separated by a porous membrane to simulate the alveolar-capillary interface. To represent the application of NF-83 composition, a 5 mL sample was dispersed with 1 mg of Texas Red DHPE triethylammonium salt, centrifuged, and sonicated in a water bath for 20 minutes. 6 μL of this solution was diluted with 1 mL of PBS and injected into the bottom channel of the StimuLung chip. To model oxygen flow and CO₂ uptake, the air sac's microchannels were perfused with a controlled flow of air, representing the O₂ flow, while the capillary channel was perfused with carbon dioxide to mimic venous blood flow. NF-83 compositions were allowed to diffuse through the porous membrane and into the capillary channel and were monitored in real time using an optical sensor and a multichannel ECHO fluorescent microscope. Computer vision and particle tracking algorithms were used to track the trajectory of individual NF-83 nanoparticles as they pass through the microchannels to elucidate oxygen and CO₂ diffusion in the system and analyze the flow and distribution of the body of nanoparticles.

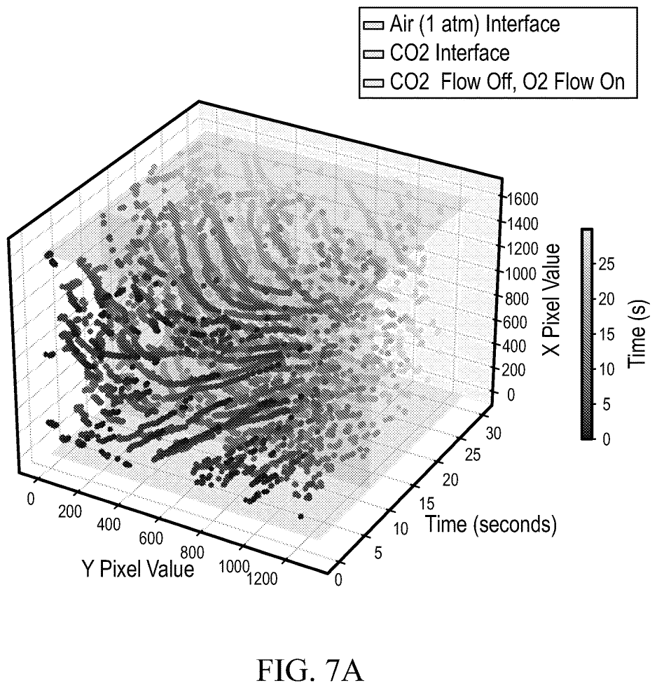

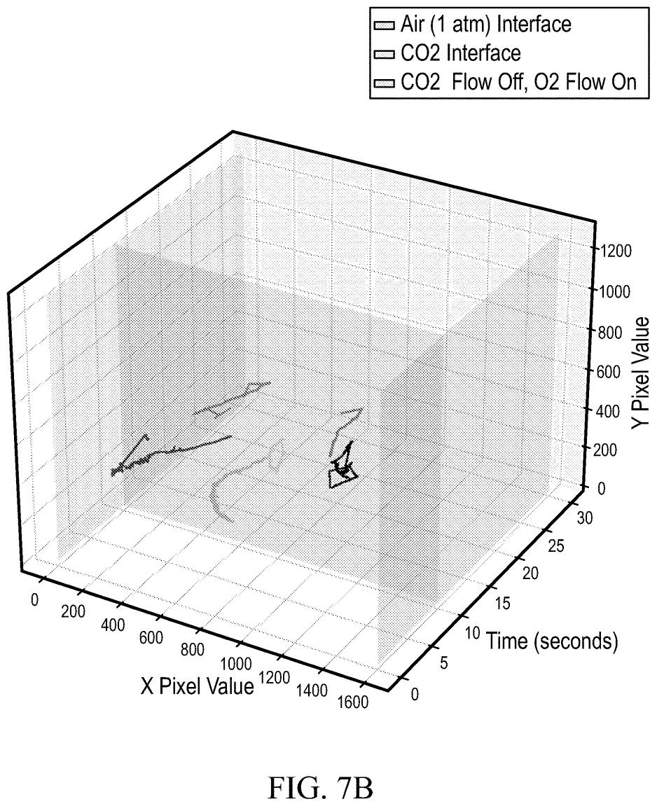

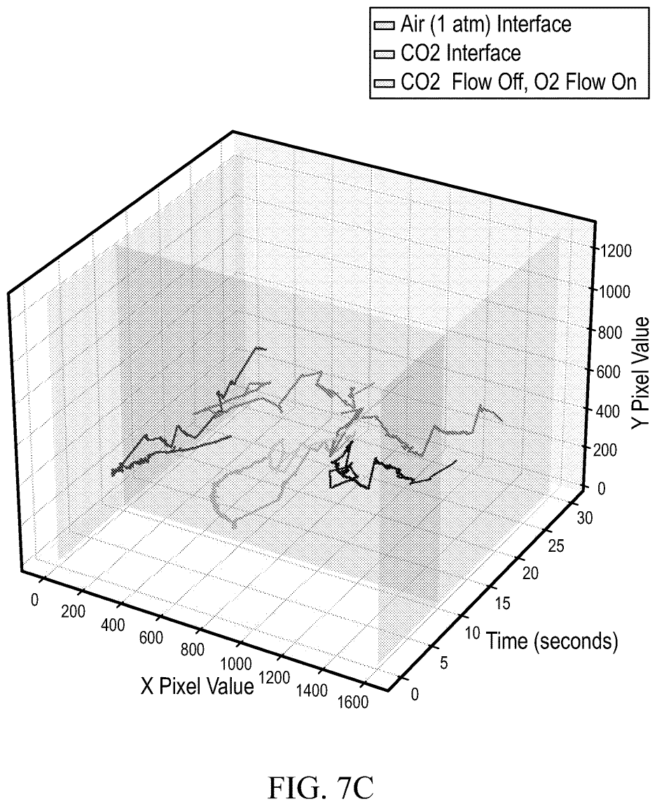

During this experiment, as shown in FIG. 7A, a “boomerang” pattern was observed in which nanoparticles that desaturated of oxygen and absorbed carbon dioxide reversed their motion, moving back towards the air interface.

Results from the computer vision-based particle tracking methods collectively demonstrated that introducing a flow of gas at the surface of the nanoparticle composition would effectively induce oscillatory behavior, leveraging the nanoparticles to stir the fluid.

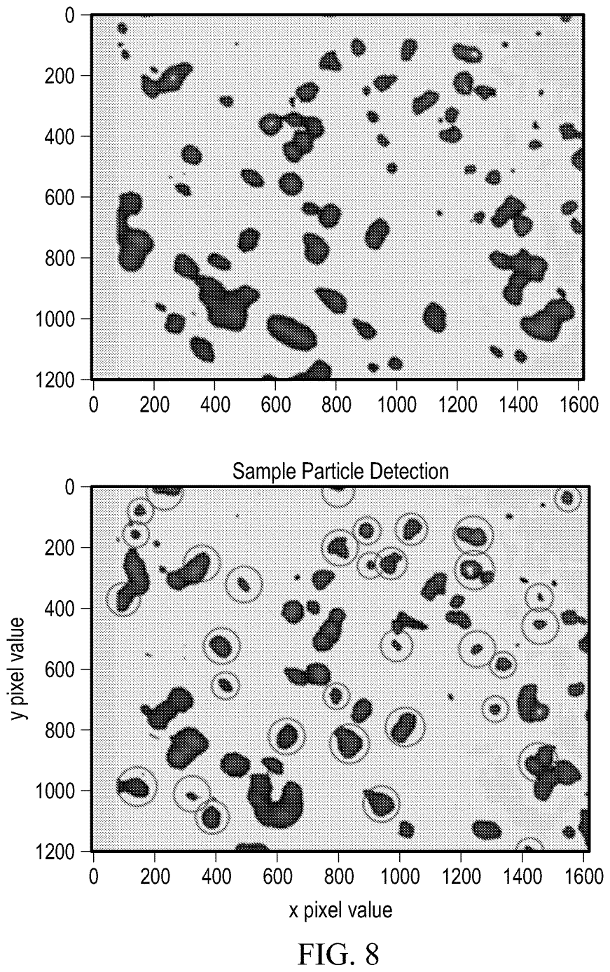

In addition to analyzing the particles' behavior in aggregate, the trajectory of individual nanoparticles was also analyzed to describe their behavior. As shown in FIG. 7B (nanoparticle trajectory 0-10 sec) and FIG. 7C (nanoparticle trajectory 0-30 sec), the nanoparticles approached the carbon dioxide interface and then started to move back towards the air interface. This phenomenon and generally similar pattern were observed at different times with different particles within the same medium and space. FIG. 8 shows sample images of the nanoparticles.

### Example 18. Spread of Exemplary Nanoparticle Composition into Alveolar Spaces

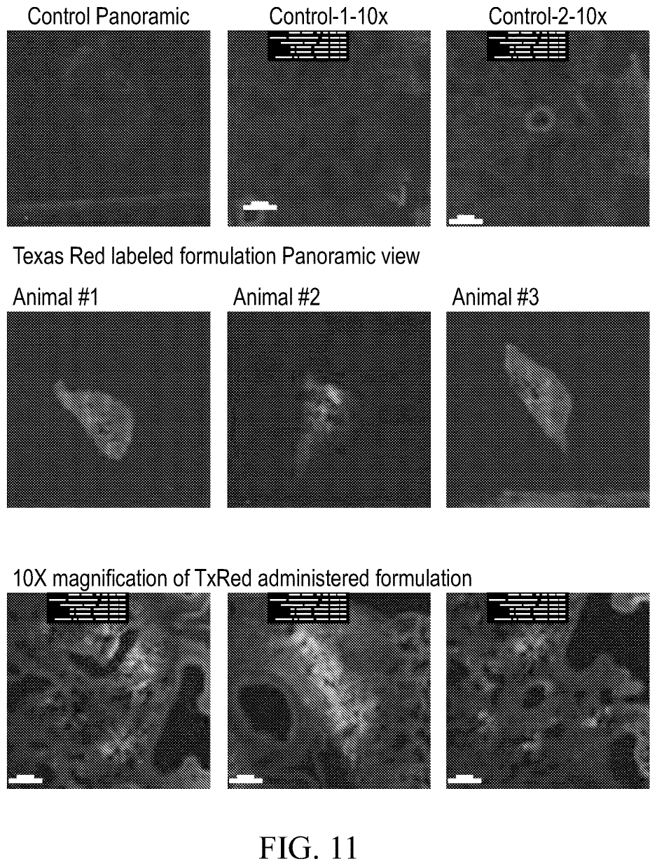

5 weight percent of the calculated total lipid content of Texas Red-X labelled lipid was mixed with the exemplary nanoparticle composition NF-83 on shaker overnight. 100 μL of this solution was given as intratracheal injection to (n=3) male mice (C57BL/6J, Jacson Labs). After few hours, the exemplary nanoparticle composition NF-83 was instilled into the lungs and the animals (three mice) were sacrificed and sectioned. The control was administered with 100 μL citrate buffer control without the labeled composition. Then the distribution of the composition was imaged. As shown in FIG. 11, the exemplary nanoparticle composition NF-83 was able to reach alveolar spaces evenly across the lung. This demonstrated the potential of the exemplary nanoparticle composition NF-83 in treating lung-related diseases.

### Example 19. Quenches of Fluoresin (FITC)

Fluorescein (FITC) is a common fluorescent reagent for biological research, with good fluorescence and water solubility. When it interacts with oxygen, it is quenched.

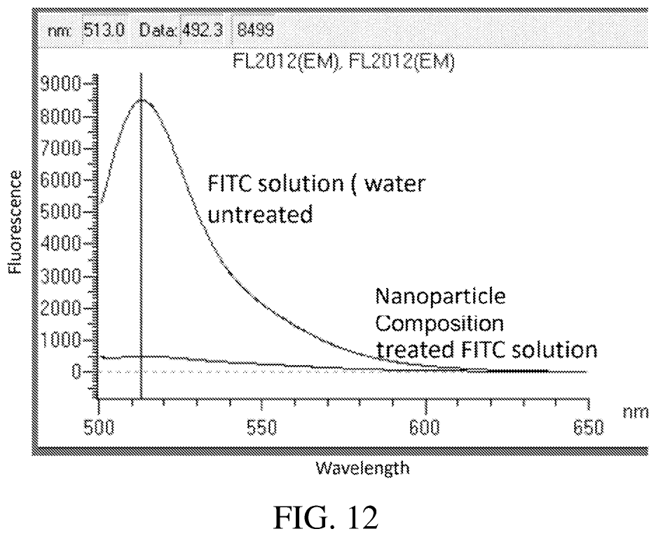

FITC solution samples were treated with the exemplary nanoparticle composition NF-83 and the FITC samples were quenched (FIG. 12). This demonstrated the oxygen carrying characteristics of the exemplary nanoparticle composition NF-83.

### Example 20. Change of Collagen Conformation

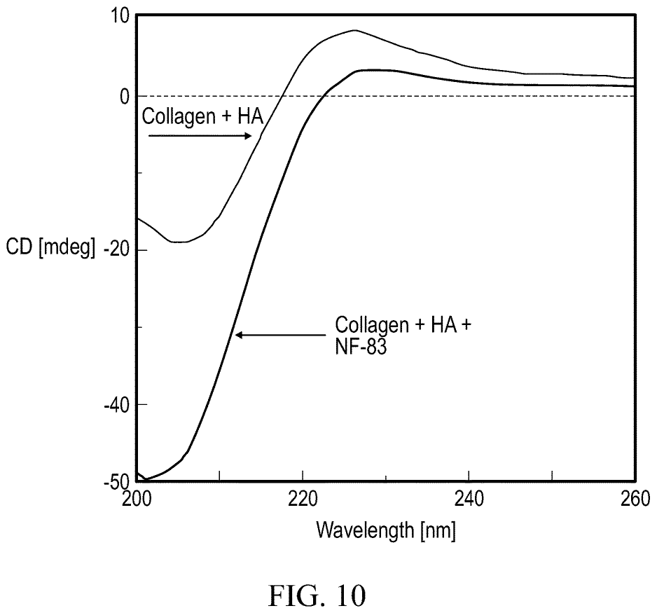

The exemplary nanoparticle composition NF-83 was prepared and tested for its ability to tune collagen conformation. The circular dichroism absorbance results are shown in FIG. 10. As shown in FIG. 10, NF-83 caused conformational changes in collagen that made it more permeable, enabling the fluid to pass through extracellular structures and fibrotic tissue. HA refers to hyaluronic acid.

### ENUMERATED EMBODIMENTS

Embodiment 1. A nano-emulsion composition for oxygen delivery, comprising nanoparticles, wherein the nanoparticles comprise a perfluorocarbon component and a lipid component, wherein the perfluorocarbon component comprises perfluorodecalin (PFD) and perflurotripropylamine (PFTPA), and optionally wherein the nano-emulsion composition comprises no more than 22% (w/w) any polymeric surfactant of the total nano-emulsion composition.

Embodiment 2. The nano-emulsion composition of embodiment 1, wherein the perfluorocarbon component consists essentially of PFD and PFTPA.

Embodiment 3. The nano-emulsion composition of embodiments 1 or 2, wherein the perfluorocarbon component is a mixture perfluorodecalin (PFD) and perflurotripropylamine (PFTPA).

Embodiment 4. The nano-emulsion composition of embodiment 3, wherein the PFD and PFTPA are at a weight ratio of about 2:1 to about 3:1.

Embodiment 5. The nano-emulsion composition of embodiments 3 or 4, wherein the PFD and PFTPA are at a weight ratio of about 2.4:1.

Embodiment 6. The nano-emulsion composition of any one of embodiments 1-5, wherein the lipid component comprises distearoylphosphatidylcholine (DSPC) and dipalmitoylphosphatidylcholine (DPPC).

Embodiment 7. The nano-emulsion composition of any one of embodiments 1-6, wherein the lipid component is a mixture of DSPC and DPPC at a weight ratio of about 1:1 to about 4:1.

Embodiment 8. The nano-emulsion composition of any one of embodiments 1-7, wherein the lipid component is a mixture of DSPC and DPPC at a weight ratio of about 2.8:1.

Embodiment 9. The nano-emulsion composition of any one of embodiments 1-8, wherein the lipid component comprises no more than 2% (w/w) hydrogenated soya phosphatidyl choline (HSPC) of the total nano-emulsion composition.

Embodiment 10. The nano-emulsion composition of any one of embodiments 1-9, further comprising a buffer.

Embodiment 11. The nano-emulsion composition of embodiment 10, wherein the buffer solution is a citrate at 50 mM.

Embodiment 12. The nano-emulsion composition of embodiments 10 or 11, wherein the buffer is about 40% to about 60% (w/w) of the total nano-emulsion composition.

Embodiment 13. The nano-emulsion composition of any one of embodiments 10-12, wherein the buffer is about 48% (w/w) of the total nano-emulsion composition.

Embodiment 14. The nano-emulsion composition of any one of embodiments 1-13, wherein nano-emulsion composition comprises no more than 2% (w/w) phosphate buffer of the total nano-emulsion composition.

Embodiment 15. The nano-emulsion composition of any one of embodiments 1-14, wherein the perfluorocarbon component is about 40% to about 60% (w/w) of the total nano-emulsion composition.

Embodiment 16. The nano-emulsion composition of any one of embodiments 1-15, wherein the perfluorocarbon component is about 50% (w/w) of the total nano-emulsion composition.

Embodiment 17. The nano-emulsion composition of any one of embodiments 1-16, wherein the lipid component is about 1.5% to about 3.5% (w/w) of the total nano-emulsion composition.

Embodiment 18. The nano-emulsion composition of any one of embodiments 1-17, wherein the lipid component is about 2.3% (w/w) of the total nano-emulsion composition.

Embodiment 19. The nano-emulsion composition of any one of embodiments 1-18, wherein the perfluorocarbon component and lipid component are at a weight ratio of about 10:1 to about 30:1.

Embodiment 20. The nano-emulsion composition of any one of embodiments 1-19, wherein the perfluorocarbon component and lipid component are at a weight ratio of about 23:1.

Embodiment 21. The nano-emulsion composition of any one of embodiments 1-20, wherein the perfluorocarbon component and buffer solution are at a weight ratio of about 0.8:1 to about 1.5:1.

Embodiment 22. The nano-emulsion composition of any one of embodiments 1-21, wherein the perfluorocarbon component and buffer solution are at a weight ratio of about 1.1:1.

Embodiment 23. The nano-emulsion composition of any one of embodiments 1-22, having a pH of about 3.5 to about 8.0.

Embodiment 24. The nano-emulsion composition of any one of embodiments 1-23, having a pH of about 6.0 to about 7.5.

Embodiment 25. The nano-emulsion composition of any one of embodiments 1-24, having a viscosity of no more than 100 cp, as determined by viscometer.

Embodiment 26. The nano-emulsion composition of any one of embodiments 1-25, having a viscosity of no more than 20 cp, as determined by viscometer.

Embodiment 27. The nano-emulsion composition of any one of embodiments 1-26, wherein the average particle size of the nanoparticles is no more than about 3 μm.

Embodiment 28. The emulsion composition of any one of embodiments 1-27, wherein the average particle size of the nanoparticles is about 100 nm to about 500 nm.

Embodiment 29. The nano-emulsion composition of any one of embodiments 1-28, wherein the polydispersity index (PDI) of the nanoparticles is about 0.5 to about 1.

Embodiment 30. The nano-emulsion composition of any one of embodiments 1-29, wherein the nanoparticles, when diluted with 5× (v/v) citrate buffer, have an average particle size of no more than 500 nm.

Embodiment 31. The nano-emulsion composition of embodiment 30, wherein the nanoparticles, when diluted with 5× (v/v) citrate buffer, have a PDI of about 0.5 to about 1.

Embodiment 32. The nano-emulsion composition of any one of embodiments 27-31, wherein the average particle size of the nanoparticles is determined by dynamic light scattering.

Embodiment 33. The nano-emulsion composition of any one of embodiments 1-31, wherein the nanoparticles have a zeta potential of about 0.02 mV to about 0.7 mV.

Embodiment 34. The nano-emulsion composition of any one of embodiments 1-33, wherein the nanoparticles have a multi lamellar structure comprising at least two lamellae layers.

Embodiment 35. The nano-emulsion composition of embodiment 34, wherein each lamellae layer comprises the perfluorocarbon sandwiched by two layers of the lipid.

Embodiment 36. The nano-emulsion composition of any one of embodiments 1-35, enriched with dissolved oxygen.

Embodiment 37. The nano-emulsion composition of any one of embodiments 1-36, enriched with dissolved oxygen at at least about 5 ppm.

Embodiment 38. The nano-emulsion composition of any one of embodiments 1-37, enriched with dissolved oxygen at about 7 ppm to about 10 ppm.

Embodiment 39. The nano-emulsion composition of any one of embodiments 36-38, wherein the concentration of the dissolved oxygen is determined by oxygen probe.

Embodiment 40. The nano-emulsion composition of any one of embodiments 1-39, capable of binding with CO₂.

Embodiment 41. The nano-emulsion composition of embodiment 40, wherein the nano-emulsion composition can carry CO₂ and be exhaled.

Embodiment 42. The nano-emulsion composition of any one of embodiments 1-41, stable for at least 4 days under room temperature without phase separation or chemical oxidation.

Embodiment 43. The nano-emulsion composition of any one of embodiments 1-42, stable for at least 10 days under room temperature without phase separation or chemical oxidation.

Embodiment 44. The nano-emulsion composition of any one of embodiments 1-43, comprising no more than about 2% (w/w) centrimonium bromide (CTAB) of the total nano-emulsion composition.

Embodiment 45. The nano-emulsion composition of any one of embodiments 1-44, comprising no more than 2% (w/w) Tween 80 of the total nano-emulsion composition.

Embodiment 46. The nano-emulsion composition of any one of embodiments 1-45, comprising no more than about 2% (w/w) Soya Lecithin of the total nano-emulsion composition.

Embodiment 47. The nano-emulsion composition of any one of embodiments 1-46, comprising no more than about 2% (w/w) poloxamer of the total nano-emulsion composition.

Embodiment 48. The nano-emulsion composition of any one of embodiments 1-46, comprising no more than about 1% (w/w) poloxamer of the total nano-emulsion composition.

Embodiment 49. The nano-emulsion composition of any one of embodiments 1-48, comprising no more than about 2% (w/w) any other surfactant or lipid component other than DSPC and DPPC of the total nano-emulsion composition.

Embodiment 50. The nano-emulsion composition of any one of embodiments 1-49, suitable for inhalation, topical administration, or enema administration.

Embodiment 51. The nano-emulsion composition of any one of embodiments 1-50, comprising:
(i) PFD at a weight percentage of about 25% to about 40% (e.g., about 35.5%) of the total nano-emulsion composition;
(ii) PFTPA at a weight percentage of about 10% to 20% (e.g., about 14.5%) of the total nano-emulsion composition;
(iii) DPPC at a weight percentage of about 0.1% to 1% (e.g. about 0.6%) of the total nano-emulsion composition;
(iv) DSPC at a weight percentage of about 1.5% to 2.5% (e.g., about 1.7%) of the total nano-emulsion composition; and
(v) pH 6.0 Citrate buffer (50 mM) at a weight percentage of about 35% to about 55% (e.g., about 47.7%) of the total nano-emulsion composition.

Embodiment 52. The nano-emulsion composition of any one of embodiments 1-51, wherein when diluted with 5× citrate buffer, the average particle size is about 200 nm to about 500 nm.

Embodiment 53. The nano-emulsion composition of any one of embodiments 1-51, wherein when diluted with 5× citrate buffer, the average particle size is about 200 nm to about 300 nm.

Embodiment 54. A nano-emulsion composition prepared by mixing about 35.5% (w/w) PFD, about 14.5% (w/w) PFTPA, about 0.6% (w/w) DPPC, about 1.7% (w/w) DSPC, and about 47.7% (w/w) pH 6.0 Citrate buffer (50 mM).

Embodiment 55. The nano-emulsion composition of embodiment 54, wherein the mixing is carried out in a microfluidizer.

Embodiment 56. The nano-emulsion composition of embodiment 55, wherein the mixing is carried out in a microfluidizer at at least about 1500 PSI for 2 to 6 cycles.

Embodiment 57. The nano-emulsion composition of any one of embodiments 1-56, suitable for nebulization and inhalation.

Embodiment 58. The nano-emulsion composition of embodiment 57, wherein before the nanoparticles arrive at alveolar epithelial cells, the nano-emulsion composition is enriched with O₂, and after contacting with the alveolar epithelial cells, the nano-emulsion composition is enriched with CO₂.

Embodiment 59. The nano-emulsion composition of embodiment 57 or 58, wherein the nano-emulsion composition can be exhaled.

Embodiment 60. A wound healing composition comprising the nano-emulsion composition of any one of embodiments 1-56 silver dioxide, and a collagen.

Embodiment 61. The composition of embodiment 60, wherein the collagen is a hydrolyzed type 1 bovine collagen or a porous type 1 bovine collagen.

Embodiment 62. The composition of embodiment 60, wherein the collagen is processed to have fetal skin-like collagen structures.

Embodiment 63. The composition of any one of embodiments 60-52, wherein the nano-emulsion composition is about 55% (w/w) of the total wound healing composition.

Embodiment 64. The composition of any one of embodiments 60-63, wherein the silver dioxide is about 1% (w/w) of the total wound healing composition.

Embodiment 65. The composition of any one of embodiments 60-64, wherein the collagen is about 15% (w/w) to about 44% (w/w) of the total wound healing composition.

Embodiment 66. An enema gel comprising the nano-emulsion composition of any one of embodiments 1-56, a poloxamer, and a PBS solution.

Embodiment 67. The gel of embodiments 66, wherein the nano-emulsion composition is about 40% (w/w) of the total enema gel.

Embodiment 68. The gel of embodiments 66 or 67, wherein the poloxamer is P407 poloxamer or P188 poloxamer.

Embodiment 69. The gel of any one of embodiments 66-68, wherein the poloxamer is P407 poloxamer at a weight percentage of about 17% to about 25% of the total enema gel.

Embodiment 70. The gel of any one of embodiments 66-69, wherein the poloxamer is P407 poloxamer at a weight percentage of about 25% of the total enema gel.

Embodiment 71. The gel of embodiments 70, wherein PBS solution is about 35% (w/w) of the total enema gel.

Embodiment 72. The gel of any one of embodiments 66-68, wherein the poloxamer is P188 poloxamer at a weight percentage of about 20% to about 50% of the total enema gel.

Embodiment 73. The gel of any one of embodiments 66-68 or 72, wherein the poloxamer is P188 poloxamer at a weight percentage of about 50% of the total enema gel.

Embodiment 74. The gel of embodiments 73, wherein PBS solution is about 10% (w/w) of the total enema gel.

Embodiment 75. A cream composition comprising the nano-emulsion composition of any one of embodiments 1-56, and a pharmaceutically acceptable excipient.

Embodiment 76. The cream composition of embodiment 75, wherein the pharmaceutically acceptable excipient comprises petroleum jelly.

Embodiment 77. The cream composition of embodiment 76, wherein the nano-emulsion composition is presented at a weight percentage of about 40% of the total cream composition.

Embodiment 78. The cream composition of embodiments 76 or 77, wherein the petroleum jelly is about 60% (w/w) of the total cream composition.

Embodiment 79. The cream composition of embodiment 75, wherein the pharmaceutically acceptable excipient comprises a colloidal oatmeal and a purified coconut oil.

Embodiment 80. The cream composition of embodiment 79, wherein the nano-emulsion composition is about 40% (w/w) to about 60% (w/w) of the total cream composition.

Embodiment 81. The cream composition of embodiments 79 or 80, wherein the colloidal oatmeal is about 20% (w/w) to about 30% (w/w) of the total cream composition.

Embodiment 82. The cream composition of any one of embodiments 79-81, wherein the purified coconut oil is about 20% (w/w) to about 30% (w/w) of the total cream composition.

Embodiment 83. The cream composition of embodiment 75, wherein the pharmaceutically acceptable excipient comprises a colloidal chaga extract and a Tinder Fungus extract.

Embodiment 84. The cream composition of embodiment 83, wherein the nano-emulsion composition is about 40% (w/w) to about 60% (w/w) of the total cream composition.

Embodiment 85. The cream composition of embodiments 83 or 84, wherein the colloidal chaga extract is about 20% (w/w) to about 30% (w/w) of the total cream composition.

Embodiment 86. The cream composition of any one of embodiments 83-85, wherein the Tinder Fungus extract is about 20% (w/w) to about 30% (w/w) of the total cream composition.

Embodiment 87. A method of delivering oxygen to a tissue of a subject in need thereof, comprising administering the nano-emulsion composition of any one of embodiments 1-59, the wound healing composition of any one of embodiments 60-65, the enema gel of any one of embodiments 66-74, or the cream composition of any one of embodiments 75-86, to the subject.

Embodiment 88. The method of embodiment 87, wherein the tissue is at the skin.

Embodiment 89. The method of embodiment 87, wherein the tissue is in the colon or GI.

Embodiment 90. The method of embodiment 89, wherein the tissue is in the lung.

Embodiment 91. The method of any one of embodiment 87 to 91, wherein the method treats hypoxemia.

Embodiment 92. A method of treating a topical condition in a subject in need thereof, comprising administering the nano-emulsion composition of any one of embodiments 1-59, the wound healing composition of any one of embodiments 60-65, the enema gel of any one of embodiments 66-74, or the cream composition of any one of embodiments 75-86, to the subject.

Embodiment 93. The method of embodiment 92, wherein the topical condition is a wound, an open injury, a burn, a colon or GI condition, or a pulmonary condition.

Embodiment 94. The method of embodiment 93, wherein the pulmonary condition is COVID, chronic obstructive pulmonary disease (COPD), asthma, or lung injury

### ENUMERATED EMBODIMENTS

Embodiment A1. A nano-emulsion composition for oxygen delivery, comprising nanoparticles, wherein the nanoparticles comprise a perfluorocarbon component and a lipid component, wherein the perfluorocarbon component comprises perfluorodecalin (PFD) and perflurotripropylamine (PFTPA), and optionally wherein the nano-emulsion composition comprises no more than about 2% (w/w) polymeric surfactant of the total nano-emulsion composition.

Embodiment A2. The nano-emulsion composition of embodiment A1, wherein the perfluorocarbon component is a mixture perfluorodecalin (PFD) and perflurotripropylamine (PFTPA), optionally at a weight ratio of about 2:1 to about 3:1.

Embodiment A3. The nano-emulsion composition of embodiments A1 or A2, wherein the lipid component is a mixture of DSPC and DPPC, optionally at a weight ratio of about 1:1 to about 4:1.

Embodiment A4. The nano-emulsion composition of any one of embodiments A1-A3, wherein the lipid component comprises no more than about 2% (w/w) hydrogenated soya phosphatidyl choline (HSPC) of the total nano-emulsion composition.

Embodiment A5. The nano-emulsion composition of any one of embodiments A1-A4, further comprising a buffer.

Embodiment A6. The nano-emulsion composition of embodiment A5, wherein the buffer is a citrate at about 20 mM to about 100 mM, optionally at a weight percentage of about 40% to about 60% (w/w) of the total nano-emulsion composition.

Embodiment A7. The nano-emulsion composition of any one of embodiments A1-A6, wherein the nano-emulsion composition comprises no more than about 2% (w/w) phosphate buffer of the total nano-emulsion composition.

Embodiment A8. The nano-emulsion composition of any one of embodiments A1-A7, wherein the perfluorocarbon component is about 40% to about 60% (w/w) of the total nano-emulsion composition.

Embodiment A9. The nano-emulsion composition of any one of embodiments A1-A8, wherein the lipid component is about 1.5% to about 3.5% (w/w) of the total nano-emulsion composition.

Embodiment A10. The nano-emulsion composition of any one of embodiments A1-A9, having a pH of about 3.5 to about 8.0.

Embodiment A11. The nano-emulsion composition of any one of embodiments A1-A10, having a viscosity of no more than about 100 cp.

Embodiment A12. The nano-emulsion composition of any one of embodiments A1-A11, wherein the average particle size of the nanoparticles is no more than about 3 μm.

Embodiment A13. The nano-emulsion composition of any one of embodiments A1-A12, wherein the nanoparticles, when diluted with 5× (v/v) citrate buffer, have an average particle size of no more than about 500 nm.

Embodiment A14. The nano-emulsion composition of any one of embodiments A1-A13, wherein the nanoparticles have a multi lamellar structure comprising at least two lamellae layers, wherein each lamellae layer comprises the perfluorocarbon sandwiched by two layers of the lipid.

Embodiment A15. The nano-emulsion composition of any one of embodiments A1-A14, enriched with at least about 5 ppm dissolved oxygen.

Embodiment A16. The nano-emulsion composition of any one of embodiments A1-A15, stable for at least 4 days under room temperature without phase separation.

Embodiment A17. The nano-emulsion composition of any one of embodiments A1-A16, comprising no more than about 2% (w/w) centrimonium bromide (CTAB) of the total nano-emulsion composition.

Embodiment A18. The nano-emulsion composition of any one of embodiments A1-A17, comprising no more than about 2% (w/w) Tween 80 of the total nano-emulsion composition.

Embodiment A19. The nano-emulsion composition of any one of embodiments A1-A18, comprising no more than about 2% (w/w) Soya Lecithin of the total nano-emulsion composition.

Embodiment A20. The nano-emulsion composition of any one of embodiments A1-A19, comprising no more than about 2% (w/w) poloxamer of the total nano-emulsion composition.

Embodiment A21. The nano-emulsion composition of any one of embodiments A1-A20, comprising no more than about 2% (w/w) surfactant or lipid other than DSPC and DPPC.

Embodiment A22. The nano-emulsion composition of any one of embodiments A1-A21, suitable for inhalation, topical administration, or enema administration.

Embodiment A23. The nano-emulsion composition of any one of embodiments A1-A22, comprising:
(i) PFD at a weight percentage of about 35.5% of the total nano-emulsion composition;
(ii) PFTPA at a weight percentage of about 14.5% of the total nano-emulsion composition;
(iii) DPPC at a weight percentage of about 0.6% of the total nano-emulsion composition;
(iv) DSPC at a weight percentage of about 1.7% of the total nano-emulsion composition; and
(v) pH 6.0 Citrate buffer (50 mM) at a weight percentage of about 47.7% of the total nano-emulsion composition.

Embodiment A24. A wound healing composition comprising the nano-emulsion composition of any one of embodiments A1-A23, silver dioxide, and a collagen.

Embodiment A25. The composition of embodiment A24, wherein the collagen is a hydrolyzed type 1 bovine collagen, optionally at a weight percentage of about 44% (w/w) of the total wound healing composition, or a porous type 1 bovine collagen, optionally at a weight percentage of about 15% (w/w) of the total wound healing composition.

Embodiment A26. The composition of embodiment A24 or A25, wherein the nano-emulsion composition is about 30% to about 90% (w/w) of the total wound healing composition.

Embodiment A27. The composition of any one of embodiments A20-A26, wherein the silver dioxide is about 1% (w/w) of the total wound healing composition.

Embodiment A28. An enema gel comprising the nano-emulsion composition of any one of embodiments A1-A23, a poloxamer, and a PBS solution.

Embodiment A29. The gel of embodiments A28, wherein the nano-emulsion composition is about 40% (w/w) of the total enema gel.

Embodiment A30. The gel of embodiments A28 or A29, wherein the poloxamer is P407 poloxamer, optionally at a weight percentage of about 17% to about 25% of the total enema gel, or P188 poloxamer, optionally at a weight percentage of about 20% to about 50% of the total enema gel.

Embodiment A31. The gel of any one of embodiments A28-A30, wherein PBS solution is about 10% (w/w) to about 35% (w/w) of the total enema gel.

Embodiment A32. A cream composition comprising the nano-emulsion composition of any one of embodiments A1-A23, and a pharmaceutically acceptable excipient.

Embodiment A33. The cream composition of embodiment A32, wherein the pharmaceutically acceptable excipient comprises petroleum jelly, optionally at a weight percentage of 60% (w/w) of the total cream composition, and the nano-emulsion composition is present at a weight percentage of about 40% of the total cream composition.

Embodiment A34. The cream composition of embodiment A32, wherein the pharmaceutically acceptable excipient comprises a colloidal oatmeal, optionally at a weight percentage of is about 20% (w/w) to about 30% (w/w) of the total cream composition, and a purified coconut oil, optionally at a weight percentage of is about 20% (w/w) to about 30% (w/w) of the total cream composition.

Embodiment A35. The cream composition of embodiment A34, wherein the nano-emulsion composition is about 40% (w/w) to about 60% (w/w) of the total cream composition.

Embodiment A36. The cream composition of embodiment A32, wherein the pharmaceutically acceptable excipient comprises a colloidal chaga extract, optionally at a weight percentage of is about 20% (w/w) to about 30% (w/w) of the total cream composition, and a Tinder Fungus extract, optionally at a weight percentage of is about 20% (w/w) to about 30% (w/w) of the total cream composition.

Embodiment A37. The cream composition of embodiment A36, wherein the nano-emulsion composition is about 40% (w/w) to about 60% (w/w) of the total cream composition.

Embodiment A38. A method of delivering oxygen to a tissue of a subject in need thereof, comprising administering the nano-emulsion composition of any one of embodiments A1-A23, the wound healing composition of any one of embodiments A24-A27, the enema gel of any one of embodiments A28-A31, or the cream composition of any one of embodiments A32-A37, to the subject.

Embodiment A39. The method of embodiment A38, wherein the tissue is at the skin, in the colon or GI, or in the lung.

Embodiment A40. A method of treating a condition in a subject in need thereof, comprising topically administering the nano-emulsion composition of any one of embodiments A1-A23, the wound healing composition of any one of embodiments A24-A27, the enema gel of any one of embodiments A28-A31, or the cream composition of any one of embodiments A32A-37, to the subject.

Embodiment A41. The method of embodiment A40, wherein the condition is a wound, an open injury, a burn, a colon or GI condition, or a pulmonary condition.

Embodiment A42. The method of embodiment A41, wherein the pulmonary condition is COVID, chronic obstructive pulmonary disease (COPD), asthma, or lung injury.

1. A nanoparticle composition for oxygen delivery, comprising nanoparticles, wherein the nanoparticles comprise a perfluorocarbon component and a lipid component, wherein the perfluorocarbon component comprises perfluorodecalin (PFD) and perflurotripropylamine (PFTPA), and optionally wherein the nanoparticle composition comprises no more than about 2% (w/w) polymeric surfactant.

2. The nanoparticle composition of claim 1, wherein the perfluorocarbon component comprises perfluorodecalin (PFD) and perflurotripropylamine (PFTPA), optionally at a weight ratio of about 2:1 to about 3:1.

3. The nanoparticle composition of claim 1 or 2, wherein the lipid component comprises DSPC and DPPC, optionally at a weight ratio of about 1:1 to about 4:1.

4. The nanoparticle composition of any one of claims 1-3, wherein the weight percentage of hydrogenated soya phosphatidyl choline (HSPC) in the total nanoparticle composition is no more than about 2% (w/w).

5. The nanoparticle composition of any one of claims 1-4, further comprising a buffer component.

6. The nanoparticle composition of claim 5, wherein the buffer component comprises sodium citrate hydrate and citric acid at a weight ratio of about 5:1 to about 20:1, optionally about 12:1.

7. The nanoparticle composition of claim 6, wherein the weight ratio between the perfluorocarbon component and the buffer component in the nanoparticle composition is about 3.5:1 to about 4.5:1, optionally 3.8:1.

8. The nanoparticle composition of any one of claims 1-7, wherein the perfluorocarbon component is about 40% to about 60% (w/w) of the total nanoparticle composition.

9. The nanoparticle composition of any one of claims 1-8, wherein the lipid component is about 1.5% to about 3.5% (w/w) of the total nanoparticle composition.

10. The nanoparticle composition of any one of claims 1-9, having a pH of about 3.5 to about 8.0.

11. The nanoparticle composition of any one of claims 1-10, having a viscosity of no more than about 100 cp.

12. The nanoparticle composition of any one of claims 1-11, wherein the average particle size of the nanoparticles is no more than about 3 μm.

13. The nanoparticle composition of any one of claims 1-12, wherein the nanoparticles, when diluted with 5× (v/v) citrate buffer, have an average particle size of no more than about 500 nm.

14. The nanoparticle composition of any one of claims 1-13, wherein the nanoparticles have a multi lamellar structure comprising at least two lamellae layers, wherein each lamellae layer comprises the perfluorocarbon sandwiched by two layers of the lipid.

15. The nanoparticle composition of any one of claims 1-14, enriched with at least about 5 ppm dissolved oxygen.

16. The nanoparticle composition of any one of claims 1-15, wherein the nanoparticle does not have phase separation for at least 4 days under room temperature.

17. The nanoparticle composition of any one of claims 1-16, comprising no more than about 2% (w/w) centrimonium bromide (CTAB).

18. The nanoparticle composition of any one of claims 1-17, comprising no more than about 2% (w/w) Tween 80.

19. The nanoparticle composition of any one of claims 1-18, comprising no more than about 2% (w/w) Soya Lecithin.

20. The nanoparticle composition of any one of claims 1-19, comprising no more than about 2% (w/w) poloxamer.

21. The nanoparticle composition of any one of claims 1-20, comprising no more than about 2% (w/w) surfactant or lipid other than DSPC and DPPC.

22. The nanoparticle composition of any one of claims 1-21, wherein the nanoparticle is suitable for inhalation, topical administration, or enema administration.

23. The nanoparticle composition of any one of claims 1-22, comprising:
(i) PFD at a weight percentage of about 35.5% of the total nanoparticle composition;
(ii) PFTPA at a weight percentage of about 14.5% of the total nanoparticle composition;
(iii) DPPC at a weight percentage of about 0.6% of the total nanoparticle composition;
(iv) DSPC at a weight percentage of about 1.7% of the total nanoparticle composition;
(v) citric acid at a weight percentage of about 0.1% of the total nanoparticle composition;
(vi) sodium citrate dihydrate at a weight percentage of about 1.2% of the total nanoparticle composition; and
(viii) water at a weight percentage of about 46.4% of the total nanoparticle composition.

24. A wound healing composition comprising the nanoparticle composition of any one of claims 1-23, further comprising silver dioxide and a collagen.

25. The wound healing composition of claim 24, wherein the collagen comprises a hydrolyzed type 1 bovine collagen, optionally at a weight percentage of about 44% (w/w) of the total wound healing composition, or a porous type 1 bovine collagen, optionally at a weight percentage of about 15% (w/w) of the total wound healing composition.

26. The wound healing composition of claim 24 or 25, wherein the nanoparticle composition is about 30% to about 90% (w/w) of the total wound healing composition.

27. The wound healing composition of any one of claims 24-26, wherein the silver dioxide is about 1% (w/w) of the total wound healing composition.

28. An enema gel, prepared by mixing the nanoparticle composition of any one of claims 1-23, a poloxamer and a PBS solution.

29. The enema gel of claim 28, wherein the nanoparticle composition is about 40% (w/w) of the total enema gel.

30. The enema gel of claim 28 or 29, wherein the poloxamer comprises a P407 poloxamer, optionally at a weight percentage of about 17% to about 25% of the total enema gel, or P188 poloxamer, optionally at a weight percentage of about 20% to about 50% of the total enema gel.

31. The gel of any one of claims 28-30, wherein PBS solution is about 10% (w/w) to about 35% (w/w) of the total enema gel.

32. A cream composition comprising the nanoparticle composition of any one of claims 1-23 and a pharmaceutically acceptable excipient.

33. The cream composition of claim 32, wherein the pharmaceutically acceptable excipient comprises petroleum jelly, optionally at a weight percentage of 60% (w/w) of the total cream composition, and the nanoparticle composition is present at a weight percentage of about 40% of the total cream composition.

34. The cream composition of claim 32, wherein the pharmaceutically acceptable excipient comprises a colloidal oatmeal, optionally at a weight percentage of about 20% (w/w) to about 30% (w/w) of the total cream composition, and a purified coconut oil, optionally at a weight percentage of about 20% (w/w) to about 30% (w/w) of the total cream composition.

35. The cream composition of claim 34, wherein the nanoparticle composition is about 40% (w/w) to about 60% (w/w) of the total cream composition.

36. The cream composition of claim 32, wherein the pharmaceutically acceptable excipient comprises a colloidal chaga extract, optionally at a weight percentage of about 20% (w/w) to about 30% (w/w) of the total cream composition, and a Tinder Fungus extract, optionally at a weight percentage of about 20% (w/w) to about 30% (w/w) of the total cream composition.

37. The cream composition of claim 36, wherein the nanoparticle composition is about 40% (w/w) to about 60% (w/w) of the total cream composition.

38. A method of delivering oxygen to a tissue of a subject in need thereof, comprising administering the nanoparticle composition of any one of claims 1-23, the wound healing composition of any one of claims 24-27, the enema gel of any one of claims 28-31, or the cream composition of any one of claims 32-37, to the subject.

39. The method of claim 38, wherein the tissue is skin, colon or GI, or lung.

40. A method of treating a condition associated with a tissue experiencing a reduction in normal oxygen levels in a subject, comprising topically administering the nanoparticle composition of any one of claims 1-23, the wound healing composition of any one of claims 24-27, the enema gel of any one of claims 28-31, or the cream composition of any one of claims 32-37, to the proximity of the tissue experiencing a reduction in normal oxygen levels.

41. The method of claim 40, wherein the tissue is in lung, in colon, in GI track, or on skin.

42. The method of claim 40 or 41, wherein the condition is a wound, an open injury, a burn, a colon or GI condition, or a pulmonary condition.

43. The method of claim 42, wherein the pulmonary condition is COVID, chronic obstructive pulmonary disease (COPD), asthma, or lung injury.

44. The method of claim 43, wherein the pulmonary condition is acute respiratory distress (ARDS).
